SEQUENCE LISTING (313 sequences)

SEQ ID NO: 1 - protein, 1228 aa, unidentified
  source 1..1228: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 4 - protein, 1238 aa, Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 5 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 6 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 7 - protein, 1206 aa, unidentified
  source 1..1206: note = Lachnospiraceae sp.
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 8 - protein, 1233 aa, unidentified
  source 1..1233: note = Lachnospiraceae sp.
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 9 - protein, 1227 aa, unidentified
  source 1..1227: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 10 - protein, 1264 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 11 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 12 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 13 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 14 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 15 - protein, 1484 aa, unidentified
  VARIANT 1073: note = misc_feature - Xaa can be any naturally occurring amino acid
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 16 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 17 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 18 - DNA, 3987 bp, synthetic construct
  misc_feature 1..3987: note = Synthetic polynucleotide
  source 1..3987: mol_type = other DNA
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 19 - DNA, 3987 bp, synthetic construct
  misc_feature 1..3987: note = Synthetic polynucleotide
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 20 - DNA, 3987 bp, synthetic construct
  misc_feature 1..3987: note = Synthetic polynucleotide
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 21 - DNA, 1592 bp, Medicago truncatula
  source 1..1592: mol_type = other DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 22 - DNA, 2000 bp, Zea mays
  source 1..2000: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 23 - protein, 228 aa, Rattus norvegicus
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 24 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 25 - DNA, 621 bp, Petromyzon marinus
  source 1..621: mol_type = other DNA
acagatgcagagtatgtgagaattcacgaaaagctggacatctataccttcaagaagcagttctttaacaataagaagtctgtgagccataggtgctacgtgctgttcgagctgaagagaaggggtgaaagaagggcatgtttttgggggtatgctgtgaacaagccccagtctggaactgagagaggcattcacgccgaaattttcagcatcagaaaggtggaggaatacctgagggataaccctggacagtttacaattaattggtattctagctggtctccatgcgctgactgtgccgagaagatcctggaatggtacaaccaggagctgagaggaaatggccataccctgaagatttgggcctgcaagctgtactatgaaaagaacgcaagaaatcagatcggactgtggaacctgagggataatggtgtggggctgaacgtgatggtgtccgagcactatcagtgctgtagaaagattttcattcagtcctcacataatcagctgaacgagaatagatggctggaaaagactctgaagagggctgagaagagaaggtccgaactgtcaattatgatccaggtgaagatcctgcacaccactaagtcacctgccgtg

SEQ ID NO: 26 - protein, 160 aa, synthetic construct
  REGION 1..160: note = Synthetic polypeptide
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 27 - protein, 207 aa, synthetic construct
  REGION 1..207: note = Synthetic polypeptide
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 28 - protein, 228 aa, synthetic construct
  REGION 1..228: note = Synthetic polypeptide
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 29 - protein, 162 aa, synthetic construct
  REGION 1..162: note = Synthetic polypeptide
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 30 - protein, 166 aa, Escherichia coli
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 31 - protein, 166 aa, synthetic construct
  REGION 1..166: note = Synthetic polypeptide
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 32 - protein, 166 aa, synthetic construct
  REGION 1..166: note = Synthetic polypeptide
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 33 - protein, 166 aa, synthetic construct
  REGION 1..166: note = Synthetic polypeptide
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 34 - protein, 166 aa, synthetic construct
  REGION 1..166: note = Synthetic polypeptide
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 35 - protein, 1763 aa, synthetic construct
  REGION 1..1763: note = Synthetic polypeptide
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 36 - protein, 1565 aa, synthetic construct
  REGION 1..1565: note = Synthetic polypeptide
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 37 - protein, 1565 aa, synthetic construct
  REGION 1..1565: note = Synthetic polypeptide
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 38 - protein, 364 aa, synthetic construct
  REGION 1..364: note = Synthetic polypeptide
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 39 - protein, 167 aa, synthetic construct
  REGION 1..167: note = Synthetic polypeptide
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 40 - protein, 167 aa, synthetic construct
  REGION 1..167: note = Synthetic polypeptide
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 41 - protein, 83 aa, unidentified
  source 1..83: note = Bacillus phage AR9
TNLSDIIEKETGKQLVIQESILMLPEEVEEVIGNKPESDILVHTAYDESTDENVMLLTSDAPEYKPWALVIQDSNGENKIKML

SEQ ID NO: 42-44 - skipped (3 entries)

SEQ ID NO: 45 - protein, 24 aa, synthetic construct
  REGION 1..24: note = Synthetic peptide
EELLSKNYHLENEVARLKKGSGSG

SEQ ID NO: 46 - protein, 241 aa, synthetic construct
  REGION 1..241: note = Synthetic polypeptide
EEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSG

SEQ ID NO: 47 - protein, 277 aa, synthetic construct
  REGION 1..277: note = Synthetic polypeptide
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 48 - DNA, 66 bp, Saccharomyces bayanus
  source 1..66: mol_type = other DNA
ttcttgtcgtacttatagatcgctacgttatttcaattttgaaaatctgagtcctgggagtgcgga

SEQ ID NO: 49 - protein, 605 aa, Homo sapiens
MSGWESYYKTEGDEEAEEEQEENLEASGDYKYSGRDSLIFLVDASKAMFESQSEDELTPFDMSIQCIQSVYISKIISSDRDLLAWFYGTEKDKNSVNFKIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPLKEACRAYGLKSGLKKQELLEALTKHFQD

SEQ ID NO: 50 - protein, 482 aa, synthetic construct
  REGION 1..482: note = Synthetic polypeptide
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 51 - DNA, 10 bp, Methanobacterium thermoautotrophicum
  source 1..10: mol_type = other DNA
aatttttgga

SEQ ID NO: 52 - protein, 83 aa, Methanobacterium thermoautotrophicum
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 53 - DNA, 25 bp, Bacteriophage MS2
  source 1..25: mol_type = other DNA
gcgcacatgaggatcacccatgtgc

SEQ ID NO: 54 - protein, 116 aa, Bacteriophage MS2
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 55 - DNA, 26 bp, Bacteriophage PP7
  source 1..26: mol_type = other DNA
ataaggagtttatatggaaaccctta

SEQ ID NO: 56 - protein, 127 aa, Bacteriophage PP7
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 57 - DNA, 19 bp, unidentified
  source 1..19: mol_type = other DNA; note = Shigella phage
ctgaatgcctgcgagcatc

SEQ ID NO: 58 - protein, 62 aa, unidentified
  REGION 1..62: note = Shigella phage
MKSIRCKNCNKLLFKADSFDHIEIRCPRCKRHIIMLNACEHPTEKHCGKREKITHSDETVRY

SEQ ID NO: 59 - protein, 1367 aa, synthetic construct
  REGION 1..1367: note = Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 60 - protein, 1367 aa, synthetic construct
  REGION 1..1367: note = Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 61 - DNA, 4101 bp, synthetic construct
  misc_feature 1..4101: note = Synthetic polynucleotide
  source 1..4101: mol_type = other DNA
gacaagaagtacagcatcgggctggcgatcgggaccaactccgtcggctgggctgtgattaccgacgagtacaaggtgccatccaagaagttcaaggtcctcggcaacactgaccggcacagcattaagaagaacctgattggggcgctgctgttcgattcgggggagactgcggaggcgaccaggctgaagcggactgcgcgccggaggtacaccaggaggaagaatcggatctgctacctccaggagattttctcgaatgagatggccaaggtggacgattccttcttccatcgcctggaggagtcgttcctcgttgaggaggacaagaagcatgagaggcatcccattttcgggaatatcgttgacgaggtggcttaccatgagaagtacccgaccatctaccatctgcggaagaagctcgtcgattcgaccgataaggccgacctgcggctgatctacctggccctcgcgcacatgattaagttccggggccatttcctcatcgagggcgacctcaacccggacaactcggacgtggataagctcttcattcagctcgtgcagacatacaaccagctcttcgaggagaatcccattaacgcctcgggggtcgacgctaaggctattctctcggctcggctgtcgaagtcgcgccggctggagaatctcattgcccagctcccaggcgagaagaagaacggcctcttcggcaacctgattgccctgtcgctggggctcacaccgaatttcaagtcgaacttcgacctcgccgaggacgctaagctccagctcagcaaggatacttacgatgatgacctcgataacctgctcgcccagattggggatcagtacgcggatctgttcctcgcggccaagaatctcagcgatgctattctcctgtcggacattctccgcgtcaacacagagattactaaggccccactgtcggcgagcatgattaagaggtacgatgagcatcatcaggacctgacactgctcaaggcgctggtccggcagcagctccccgagaagtacaaggagattttcttcgatcagtcaaagaatgggtacgcgggctacattgatggcggcgcgtcccaggaggagttctacaagttcattaagcccatcctggagaagatggacgggaccgaggagctgctggtgaagctcaatcgggaggacctgctccggaagcagcgcacattcgacaatggctcgattcctcaccagattcacctgggcgagctgcacgccattctccgcaggcaggaggacttctacccgttcctcaaggacaaccgcgagaagatcgagaagatcctgaccttccggattccatactacgtggggccgctcgcgcgggggaactcccggttcgcgtggatgactcgcaagtccgaagaaacgattacaccgtggaatttcgaggaggtcgtcgacaagggcgctagtgcgcagtcattcattgagaggatgaccaatttcgataagaacctgcctaacgagaaggtgctgccgaagcattcgctgctctacgagtacttcaccgtttacaatgagctgaccaaggtgaagtatgtgactgagggcatgaggaagccagcgttcctgagcggcgagcagaagaaggctatcgtggacctgctcttcaagactaaccggaaggtgactgtgaagcagctcaaggaggactacttcaagaagattgagtgcttcgattccgttgagattagcggggtggaggatcggttcaatgcttcgctcgggacataccacgatctcctgaagatcattaaggataaggacttcctcgacaacgaggagaacgaggacattctcgaagatattgtcctgaccctcaccctcttcgaggatcgggagatgatcgaggagaggctcaagacatacgctcatctgttcgatgataaggtcatgaagcagctgaagcgcaggcggtacacagggtgggggcggctgagccggaagctgatcaacgggattcgggataagcagtccgggaagacaattctcgacttcctcaagtccgacgggttcgctaaccggaacttcatgcagctcattcatgatgactcgctgacattcaaggaggatattcagaaggcgcaggtttcggggcagggcgactcgctccacgagcatattgcgaatctggcgggctcccccgcgattaagaagggcattctgcaaaccgtcaaggtggttgatgagctggtcaaggtcatggggcggcataagccagagaatattgtcatcgagatggcgcgggagaatcagaccacacagaaggggcagaagaactcacgggagcggatgaagcgcatcgaggagggcatcaaggagctggggtcgcagatcctgaaggagcatcccgtggagaacactcagctgcaaaatgagaagctgtacctctactacctccagaacgggagggacatgtatgtggatcaggagctggatattaataggctgagcgattacgatgtcgaccacattgtcccacagtcgttcctgaaggacgacagcattgacaacaaggtgctgacccgctcggataagaacaggggcaagagcgataatgttccaagcgaggaggttgtgaagaagatgaagaactactggcggcagctcctgaacgcgaagctcatcacacagcggaagttcgacaacctcaccaaggctgagcgcgggggcctgagcgagctggacaaggcggggttcattaagaggcagctggtcgagacacggcagattacaaagcatgttgcgcagattctcgattcccggatgaacaccaagtacgatgagaacgataagctgattcgggaggtcaaggtaattaccctgaagtccaagctggtgtccgacttcaggaaggacttccagttctacaaggttcgggagatcaacaactaccaccacgcgcatgatgcctacctcaacgcggtcgtggggaccgctctcatcaagaagtacccaaagctggagtcagagttcgtctacggggattacaaggtttacgacgtgcggaagatgatcgctaagagcgagcaggagattggcaaggctaccgctaagtacttcttctactccaacatcatgaacttcttcaagacagagattaccctcgcgaatggcgagatccggaagaggcccctcatcgagacaaatggggagacaggggagattgtctgggataaggggcgggatttcgcgaccgtccggaaggtcctgtcgatgccccaggttaatattgtcaagaagactgaggtccagactggcggcttctcaaaggagtcgattctcccaaagaggaactccgataagctcattgctcggaagaaggattgggaccccaagaagtacgggggattcgactcccccactgttgcttactctgttctggttgttgctaaggtggagaaggggaagtcgaagaagctgaagagcgtgaaggagctgctcgggattacaattatggagaggtcatccttcgagaagaatcccatcgacttcctggaggccaagggctacaaggaggtgaagaaggacctgattattaagctgcccaagtactcgctcttcgagctggagaatgggcggaagcggatgctggcgtccgcgggggagctgcaaaaggggaacgagctggcgctcccctccaagtatgtgaacttcctctacctggcgtcgcactacgagaagctgaaggggtccccagaggataatgagcagaagcagctcttcgtcgagcagcataagcactacctggacgagattatcgagcagattagcgagttctcgaagcgggtcatcctcgcggatgcgaacctggataaggtgctcagcgcctacaataagcaccgggacaagccgattcgggagcaggcggagaatattattcacctcttcacactcaccaacctcggggcaccagctgcgttcaagtacttcgacactactatcgaccggaagcggtacacctcgacgaaggaggtgctcgacgccaccctcattcaccagtcgatcacaggcctgtacgagacacggattgacctgtcccagctcgggggcgac

SEQ ID NO: 62 - DNA, 4101 bp, synthetic construct
  misc_feature 1..4101: note = Synthetic polynucleotide
  source 1..4101: mol_type = other DNA
gacaagaagtactccattggcctggcgattgggacaaactcggtggggtgggccgtgattacggatgagtacaaggttccaagcaagaagttcaaggtcctcgggaacacagatcggcattcgattaagaagaatctcattggggcgctcctcttcgactcgggggagacagcggaggctaccaggctcaagcggacagccaggcggcggtacacaaggcggaagaatcgcatctgctacctccaggagattttctcgaatgagatggcgaaggtggacgacagcttcttccatcggctggaggagtccttcctggtggaggaggataagaagcacgagaggcatccaattttcgggaacatcgtggacgaggttgcgtaccatgagaagtaccctacaatctaccatctgcggaagaagctggttgactccacagacaaggcggacctgaggctgatctacctcgctctggcccacatgattaagttccgcgggcatttcctgatcgagggggacctgaatcccgacaattcggatgtggacaagctcttcatccagctggtgcagacctacaaccagctgttcgaggagaatcccatcaatgcgtcgggcgttgacgctaaggccattctgtccgctaggctgtcgaagagcaggaggctggagaacctgatcgcccagctgccaggcgagaagaagaatgggctcttcgggaatctgattgcgctctccctggggctgacaccgaacttcaagagcaatttcgatctggctgaggacgcgaagctccagctctcgaaggacacttacgacgatgacctcgataacctcctcgcgcagatcggggaccagtacgctgatctcttcctcgccgctaagaacctctcggatgctatcctgctctccgacattctccgggttaataccgagattacaaaggccccactgtcggcgtccatgatcaagcggtacgatgagcatcatcaggatctcaccctgctcaaggccctcgtgcggcagcagctgcccgagaagtacaaggagattttcttcgaccagagcaagaatgggtacgctggctacattgacggcggggcctcacaggaggagttctacaagttcatcaagccaatcctggagaagatggatgggacagaggagctgctggtgaagctcaaccgggaggatctgctcaggaagcagcggacgttcgacaacgggtcgattccccatcagatccacctgggggagctgcacgcgatcctgcgccggcaggaggatttctaccctttcctgaaggataatcgggagaagatcgagaagattctcaccttccggattccctactacgtcgggccactcgcgcggggcaatagcaggttcgcctggatgacacggaagagcgaggagacaatcaccccctggaacttcgaggaggttgtcgacaagggggcgtccgcccagtcattcattgagcggatgaccaatttcgacaagaatctgccaaatgagaaggttctcccaaagcatagcctcctctacgagtacttcactgtttacaacgagctgaccaaggtgaagtatgtgaccgagggcatgcggaagcccgcgttcctgtccggcgagcagaagaaggccattgtggacctcctgttcaagaccaatcgcaaggtcacagtcaagcagctcaaggaggattacttcaagaagatcgagtgcttcgactcggttgagattagcggggtggaggatcggttcaacgcgagcctcggcacttaccacgacctcctgaagatcatcaaggataaggacttcctcgacaacgaggagaacgaggatattctggaggacatcgtgctcaccctgacgctgttcgaggatcgggagatgatcgaggagcgcctgaagacctacgctcatctcttcgatgataaggtcatgaagcagctgaagaggaggcggtacaccgggtggggccgcctgagcaggaagctcattaacgggatcagggacaagcagagcggcaagaccatcctggacttcctcaagagcgatggcttcgccaaccggaatttcatgcagctcatccacgacgactccctcaccttcaaggaggacattcagaaggctcaggtcagcggccagggcgactcgctgcatgagcacatcgctaacctggcgggcagcccagccatcaagaagggcatcctccagacagtgaaggtcgtggatgagctggtgaaggtcatgggccggcataagcccgagaatattgtgattgagatggcgcgggagaatcagaccactcagaagggccagaagaactcgcgggagcgcatgaagaggatcgaggaggggattaaggagctgggcagccagattctcaaggagcaccccgtggagaatacccagctccagaacgagaagctgtacctctactacctccagaatgggcgggacatgtatgttgatcaggagctggacatcaatcgcctctcggattacgacgtggaccacatcgtgccccagagcttcctgaaggatgatagcatcgacaataaggtcctgacccgctccgacaagaatcgcggcaagagcgacaacgtgccgagcgaggaggtcgtgaagaagatgaagaactactggcggcagctgctgaacgcgaagctcattacacagcggaagttcgataacctgacgaaggcggagaggggcggcctctccgagctggacaaggcgggcttcattaagaggcagctcgtggagactcgccagatcaccaagcacgtggctcagatcctcgatagccggatgaatacgaagtacgatgagaatgacaagctcatccgggaggtgaaggtaatcaccctgaagtcaaagctcgttagcgatttccggaaggacttccagttctacaaggtgcgggagattaacaactaccatcatgcgcacgatgcgtacctcaatgcggtggtgggcacagccctgattaagaagtaccccaagctggagagcgagttcgtctacggggactacaaggtgtacgatgttcggaagatgatcgccaagagcgagcaggagattgggaaggccaccgctaagtacttcttctactcgaatattatgaatttcttcaagaccgagatcacactcgctaatggggagattcggaagcggcccctcatcgagactaacggggagactggcgagattgtgtgggacaaggggcgcgacttcgctaccgtgcgcaaggtcctctcgatgccccaggttaatattgttaagaagacagaggtgcagacgggcgggttctccaaggagtctatcctgccgaagcggaactcggacaagctgatcgcccgcaagaaggattgggaccccaagaagtacgggggattcgatagcccaaccgtggcttacagcgtcctggtggtcgccaaggttgagaaggggaagtcgaagaagctcaagagcgttaaggagctgctgggcatcaccatcatggagcggtccagcttcgagaagaatcctatcgacttcctggaggctaaggggtacaaggaggtcaagaaggacctgatcattaagctgcccaagtactctctgttcgagctggagaacgggaggaagcggatgctggcgtctgctggcgagctacagaagggcaatgagctggcgctcccctcgaagtatgtcaacttcctctacctggcttcccattacgagaagctgaagggctcgcccgaggataatgagcagaagcagctcttcgtggagcagcacaagcactacctcgacgagatcattgagcagatttcggagttctcgaagcgggtcattctcgcggacgcgaacctcgacaaggtcctctcggcgtacaacaagcaccgggacaagcccatccgggagcaggccgagaacattatccacctcttcacactgaccaacctcggcgctcccgccgcgttcaagtacttcgacaccaccattgaccgcaagagatacacatccaccaaggaggtgctggacgcgaccctcatccaccagagcatcacaggcctctacgagacacggatcgacctctcgcagctcgggggcgat

SEQ ID NO: 63 - DNA, 4092 bp, synthetic construct
  misc_feature 1..4092: note = Synthetic polynucleotide
  source 1..4092: mol_type = other DNA
gacaagaagtactcgatcggcctggcgattggcacaaacagcgtggggtgggctgtgatcactgatgagtacaaggtgccatcgaagaagttcaaggtgctggggaatacagaccggcattcgatcaagaagaatctcattggcgctctcctcttcgattccggcgagactgctgaggcgacccgcctgaagcgcaccgcccggcggcgctacactcggcggaagaataggatttgctacctccaggagattttctcgaatgagatggccaaggtggatgacagcttcttccaccgcctggaggagtcgttcctggtcgaggaggacaagaagcatgagcggcaccctatcttcgggaatatcgttgatgaggtcgcctaccacgagaagtaccccactatctaccatctccgcaagaagctcgtggacagcacagataaggccgacctccgcctgatctacctcgccctcgcgcacatgattaagttccgggggcacttcctcattgagggggatctgaatcccgataactccgacgtggacaagctgttcatccagctggtgcagacatacaaccagctgttcgaggagaatcccatcaacgcgagcggcgtggacgctaaggccattctgtcggctaggctctcgaagtcgaggcggctggagaacctgattgcgcagctccccggcgagaagaagaacgggctgttcgggaatctcatcgccctctccctcggcctcacaccaaacttcaagagcaatttcgacctggctgaggacgctaagctgcaactctcaaaggatacatacgatgacgacctggacaatctcctggctcagatcggcgaccagtacgctgacctgttcctcgcggccaagaatctgtcggacgcgattctcctcagcgacatcctgcgcgtcaataccgagattacgaaggctccactgtctgcgtcaatgattaagcggtacgatgagcatcaccaggatctgaccctcctgaaggcgctcgtgcggcagcagctgcccgagaagtacaaggagattttcttcgatcagagcaagaatggctacgccggctacatcgacgggggcgcgagccaggaggagttctacaagttcatcaagcccatcctggagaagatggacggcaccgaggagctactcgtgaagctcaatcgggaggatctcctccggaagcagcggacattcgataacgggtctatcccacaccagatccacctcggcgagctgcatgcgattctgcggcggcaggaggatttctaccctttcctgaaggacaaccgggagaagatcgagaagatcctcacattccggattccatactacgtcggccccctggcgaggggcaatagccggttcgcgtggatgacaaggaagtccgaggagactattaccccgtggaatttcgaggaggtggttgacaagggcgcttccgcgcagagcttcattgagcggatgacaaacttcgacaagaatctccccaacgagaaggtcctgccgaagcatagcctcctgtacgagtacttcaccgtctacaatgagctaactaaggtcaagtatgtgacagagggcatgaggaagccagccttcctctcaggcgagcagaagaaggccattgtggacctcctgttcaagacaaaccgcaaggtgacagtgaagcagctgaaggaggattacttcaagaagattgagtgcttcgactcagtggagatttcaggcgtggaggatcggttcaacgcgagcctggggacttaccacgacctgctgaagattattaaggacaaggacttcctggataacgaggagaatgaggacatcctggaggatattgtgctcaccctcaccctgttcgaggacagggagatgattgaggagaggctcaagacctacgcgcacctgttcgatgacaaggtcatgaagcagctgaagaggcggcgctacactgggtggggccgcctgtcgcggaagctgatcaacggcattcgggataagcagtccgggaagaccattctggatttcctgaagtcggacggcttcgccaacaggaatttcatgcagctgatccacgacgactccctcaccttcaaggaggacattcagaaggcccaggttagcggccagggggactcactccacgagcatattgccaatctggccggctctccagctatcaagaagggcatcctgcaaacagttaaggttgttgacgagctggttaaggtcatggggcggcataagcccgagaacattgtcatcgagatggctcgggagaaccagacaactcagaagggccagaagaactccagggagcgcatgaagcggattgaggagggcattaaggagctggggtcccagatcctcaaggagcaccctgtcgagaacactcagctgcaaaacgagaagctctacctgtactacctccagaacgggcgggatatgtatgtggatcaggagctggacatcaacaggctctccgactacgacgtggatcacattgtcccacagtctttcctcaaggatgattccatcgacaacaaggtgctgacgcgcagcgacaagaatagggggaagtcggacaacgttccgagcgaggaggtcgtgaagaagatgaagaattactggaggcagctcctgaatgcgaagctgatcactcagaggaagttcgacaatctgacaaaggcggagaggggcgggctctcggagctggataaggcgggcttcatcaagcggcagctcgttgaaacccggcagatcaccaagcatgtcgcccagatcctcgatagccgcatgaacaccaagtacgatgagaacgacaagctcattcgggaggttaaggtcattacgctgaagtccaagctcgtcagcgacttcaggaaggatttccagttctacaaggttcgggagattaacaactaccaccacgcgcatgatgcgtacctgaacgctgttgtcggcactgctctcatcaagaagtacccaaagctggagtccgagttcgtctacggggactacaaggtctacgatgtccggaagatgatcgccaagtcggagcaggagatcgggaaggctactgcgaagtacttcttctacagcaacattatgaatttcttcaagacggagattacgctggcgaacggggagattaggaagaggcccctcattgagactaatggggagacaggcgagattgtttgggacaagggccgcgacttcgcgactgtgcggaaggtcctgtccatgccacaggtgaatattgttaagaagacagaggtgcagactgggggcttctcgaaggagagcattctcccaaagcggaacagcgataagctcatcgcgcgcaagaaggattgggaccctaagaagtacggcggcttcgattctcccactgtggcctactccgttctcgtggttgccaaggttgagaaggggaagtcgaagaagctgaagtcggtcaaggagctgctcgggattacaatcatggagcggagcagcttcgagaagaaccctattgatttcctggaggccaagggctacaaggaggttaagaaggatctcattatcaagctccctaagtactctctgttcgagctggagaatggccggaagaggatgctggcctcggctggcgagctacagaaggggaatgagctggccctcccgtcgaagtatgtgaatttcctgtacctcgcgtcgcactacgagaagctcaagggcagcccggaggataatgagcagaagcagctcttcgtggagcagcataagcactacctggacgagatcattgagcagatcagcgagttctcgaagcgggttattctggctgatgctaacctggacaaggttctgagcgcctacaataagcatcgcgacaagccgattcgcgagcaggcggagaatattatccacctgttcaccctcactaacctcggggctcccgcggccttcaagtacttcgataccacaatagataggaagcggtacacctcgacgaaggaggtcctcgacgccacactcatccatcagtcgattacaggcctgtacgagacacggattgacctctcgcagctg

SEQ ID NO: 64 - DNA, 4101 bp, synthetic construct
  misc_feature 1..4101: note = Synthetic polynucleotide
  source 1..4101: mol_type = other DNA
gacaagaagtattccataggcctggctatcggcaccaacagcgtgggctgggccgtcatcaccgacgagtacaaagtgccgagtaaaaagttcaaagtgctcggcaacaccgaccgccactccataaagaaaaacctgatcggggcgctcctgttcgacagcggcgagacggcggaggccacccgcttgaaacgcacggcccgacggcgctacacgcggcgcaagaaccggatctgttacctacaggagattttctctaacgagatggcgaaggtggacgactcgttctttcaccgcctcgaagagtccttcctcgtggaggaggacaagaaacacgagcgccacccgatcttcggcaacatcgtggacgaggtggcctaccacgagaagtacccgaccatctaccacctccggaagaaactcgtggacagcacggacaaggccgacctgaggctcatctacctcgccctggcgcacatgattaagttccggggccacttcctgatcgagggcgacctgaacccggacaacagcgacgtggacaagctgttcatccagctagtccagacctacaaccagcttttcgaggaaaaccccatcaacgccagcggggtggacgcgaaggcgatcctgtccgcccggctgagcaagtcccggcggctggagaacctcatcgcgcagttgcccggcgagaagaagaacgggctgttcgggaacctgatcgccctctccctggggctcaccccgaacttcaagtccaacttcgacctcgccgaggacgccaaactacagctgagcaaggacacctacgacgacgacctcgacaacctgctggcccagatcggggaccagtacgcagacctgttcctcgccgccaagaacctctccgacgccatcctgctgtcggacatcctgcgggtgaacacggagatcacgaaggccccgctctcggcctcgatgattaaacgctacgacgagcaccaccaggacttgaccctcctcaaggcgctggtccgccagcagcttcccgagaagtacaaggaaatctttttcgatcagagcaagaacgggtacgccgggtacatcgacggcggggcgtcccaggaggagttctacaagttcatcaagcccatcctggagaaaatggacgggaccgaggagctgctcgtgaagctcaaccgcgaagatttgctccgcaagcagcgcacgttcgacaacgggtcgatcccgcaccagatccacctgggcgagctgcacgcgatcctcaggcgtcaggaagacttctaccccttcctcaaggacaaccgcgagaagatagagaagattctgaccttcagaattccttattacgtgggcccgctggctcggggcaactcgcgcttcgcctggatgacgcgcaagtccgaggagaccatcaccccgtggaacttcgaggaggtggtggataagggtgcctcggcccagtccttcatcgagcggatgaccaacttcgacaagaacctgccgaacgagaaggtgctccccaagcacagcctgctctacgaatatttcacggtgtacaacgagctgacgaaggtcaagtacgtgaccgagggaatgaggaaacctgcattcctctccggggagcagaagaaagccatagtcgacctcctgttcaagaccaaccggaaggtcaccgtcaagcagctcaaggaggactacttcaagaagatcgagtgcttcgattcagtggagatcagcggcgtcgaggaccggttcaacgccagcctgggcacctaccacgacctgctcaagatcatcaaggacaaggacttcctcgacaacgaggagaacgaggacatcctggaggacatcgtgctgaccctgacgctcttcgaggaccgcgagatgatcgaggagcgcctcaagacctacgcccacctgttcgacgacaaggtgatgaagcagctcaagcggcggagatatactgggtggggccgcctctcccggaagctcattaacggtatcagggataagcagtccgggaagacgatcctcgacttcctcaagtcggacgggttcgccaaccgcaacttcatgcagctcatccacgacgactccctgacgttcaaggaggacatccagaaggcccaagtgtctggtcaaggtgactcgctccacgagcacatcgccaacctcgcgggcagcccggccatcaagaagggaatactccagaccgtcaaggtggtggacgagctggtgaaggtcatgggccgccacaagccggagaacatcgtcatcgagatggcgcgggagaaccagaccacgcagaaggggcagaaaaatagccgtgagcgcatgaagcgcatcgaggaggggattaaggagttgggcagccagatcctcaaggagcaccctgtggagaacacgcagttgcaaaacgagaagctctacctgtactacctccagaacgggagggatatgtacgtggaccaagaactggacatcaaccgcctgtccgactacgacgtggaccacatcgtgccgcagagcttcctcaaggacgacagcatcgacaacaaggtgctcacccggtccgacaagaatcggggcaagtccgacaacgtgcccagcgaggaggtcgtcaaaaagatgaaaaactactggcgacaactactgaacgccaagctcatcacccagcgcaagttcgacaacctcacaaaagccgagcgcggcgggttgagcgagctggacaaggccgggttcatcaagcgccagctcgtcgagacgcgccagatcacgaagcacgtcgcgcagatactcgacagccggatgaacaccaagtacgacgagaacgacaagctcatccgggaggtgaaggtcatcaccctcaagtcgaagctcgtgagcgacttccgcaaggacttccagttctacaaggtccgggagatcaacaactaccaccacgcccacgatgcttatcttaacgccgtggtggggacggccctcattaagaaatacccgaagctggagtcggagttcgtgtacggcgactacaaggtgtacgacgtcaggaagatgatcgccaagtccgaacaggagatcgggaaggccacggcgaaatacttcttctacagcaacatcatgaacttcttcaagaccgagatcaccctcgccaacggcgagatccgcaagcgcccgctcatcgagacgaacggggagaccggcgagatcgtctgggacaaggggcgcgacttcgccactgtgcggaaggtgctgtcgatgccccaggtcaacatcgtcaagaagacggaggtccagacgggcgggttcagcaaggagagcatcctgccgaagcgcaacagcgacaagctgatcgcccgcaaaaaggactgggatccaaaaaagtacggcggcttcgacagccccaccgtcgcctacagcgtcctcgtcgtcgctaaagtcgagaagggcaagtccaaaaagctcaagagcgtcaaggagctgctcgggatcaccatcatggagcggtccagcttcgagaagaacccaattgatttcctggaggcgaagggctacaaggaggtcaagaaagacctcatcataaagctgccgaagtactcactcttcgagctggagaacgggcgcaagcggatgctggcgtcggccggagagctccaaaagggcaacgagctggcgctgccgagcaagtacgtgaacttcctctacctggcgtcccactacgagaagctcaagggcagtccagaggataacgagcagaagcagctattcgtggagcagcacaagcactacctggacgagatcatcgagcagatcagcgagttctccaagcgcgtcatcctggcggacgccaacctggacaaggtgctgtccgcgtacaacaagcaccgcgacaagccgatccgcgagcaagccgagaacatcatccacctgttcaccctcacgaacctcggggcacccgccgccttcaaatatttcgacacgaccatcgaccgcaagcgctacaccagcacgaaggaggtgctcgacgccaccctgatccaccagagcatcaccgggctgtacgagacccgcatcgacctctcgcagctcggcggggac

SEQ ID NO: 65 - DNA, 4101 bp, synthetic construct
  misc_feature 1..4101: note = Synthetic polynucleotide
  source 1..4101: mol_type = other DNA
gacaagaagtacagtattggattggccatcgggacgaacagcgtgggctgggccgtcatcaccgacgagtacaaggtgccatccaagaagtttaaggttctggggaataccgaccgccactcgatcaagaaaaatctcatcggggcgctgcttttcgacagcggcgagacggcggaagcgacgcggctcaagcggacggctcgtcgccgttacacccggcgtaagaaccgcatctgttacctccaggagatattcagcaacgagatggcgaaggtggacgactcctttttccaccgtcttgaggagtccttcctggtcgaggaggacaagaagcacgagcgccacccgatcttcgggaacatcgtggacgaggtggcctaccacgagaagtaccccacgatctaccacctccgcaaaaaactcgtggactcaactgacaaggccgatttgaggcttatctacctcgccctcgcccacatgattaagttccgtgggcacttcctaatcgagggtgacctcaaccccgacaactctgacgtggacaagctgttcatccagcttgtgcagacctacaatcagctctttgaggagaatccgatcaacgcatctggtgtggacgcaaaggccatcctcagcgcgcggctgagcaagtctaggcggttggagaacctgatcgcccaactgcccggcgagaagaaaaatggcctcttcggcaacctgatcgccctgtcgctggggctcacgccgaacttcaagagtaactttgacctggcggaggacgctaagctccagctatctaaggacacatacgacgacgacctggacaacctgctggcccagatcggcgaccagtacgccgacctcttcctagccgccaagaacctgtccgacgccatcctcctcagcgacatcctgcgcgtgaacacggagatcacgaaggctccgctcagcgcctccatgattaagcggtacgacgagcaccaccaagacctaactttactcaaagccctcgtgcggcagcagcttcccgagaagtacaaagagatattttttgatcagtccaagaacggttatgcgggctacatcgacggcggcgcgagccaggaggagttctacaagttcatcaagcccatcctggagaagatggacggcacggaggagctgctcgtgaagctcaaccgtgaagacctcctgcgaaagcagcgaaccttcgacaacggttcgatcccgcaccagatccacctcggggagctgcacgccatcctgaggcgacaggaggacttctaccctttcctaaaggacaaccgcgagaagattgaaaaaatcctgacgtttcgcataccctactacgtcggcccgctggcgcgcggcaactcccggttcgcctggatgacccgtaagagcgaggagacgatcaccccgtggaacttcgaggaggtcgtggacaagggcgcgagcgcgcagagcttcatcgagcgcatgaccaacttcgacaagaacctcccgaacgagaaggtgctcccaaagcactccctcctgtacgagtatttcaccgtgtacaacgagttgacaaaggtgaagtacgtgacggagggaatgcggaagcctgcgttcctctcgggcgagcagaagaaggcaatcgtggacctgctcttcaagaccaaccggaaggtgacggtgaagcagctcaaggaggactacttcaaaaaaatcgagtgcttcgactccgtggagataagcggcgtggaggaccgattcaacgcctccctcggcacctaccacgacctccttaagatcatcaaggacaaggacttcctggacaacgaggagaacgaggacatcctggaggacatcgtgctcaccctgaccctcttcgaggaccgggagatgatcgaggagcgcctcaagacgtacgcccacttgttcgacgacaaggtgatgaagcagctcaagcggcggcgatacaccgggtggggccgcctatcccgcaaacttatcaacggcatccgcgacaagcagtccggcaagacgatcctggatttcctcaagtcggacgggttcgccaaccggaacttcatgcagctcatccacgacgacagcctcacgttcaaggaggacatccagaaggcccaagtgagcggtcaaggggacagcctccacgagcacattgcgaaccttgctgggagccctgcgatcaagaaggggatattgcaaaccgtgaaggtcgtggacgagttggtgaaggtcatggggcgacacaagcccgagaacatcgtgatcgagatggccagggaaaatcagaccacgcagaagggccaaaaaaacagccgcgagcggatgaagcggatcgaggagggcatcaaggagctggggtcgcagatcctcaaggagcacccggtggagaacacgcagctccagaacgagaagctgtacctctattacctacagaacgggcgggatatgtacgtggaccaggagctagacatcaaccgcctgtccgactacgacgtggaccatatcgtcccgcagtcgttcttgaaggacgacagcatcgacaacaaggtgctcacaagatcggataagaatcgaggcaagtccgacaacgtgccctcggaggaggtggtcaagaaaatgaaaaactactggcggcagttgctgaacgccaagctcattacgcagcggaagttcgacaacctgacgaaggctgaacgtggtgggctcagcgagctagacaaggcggggttcatcaagcggcagctcgtcgagacccggcagatcaccaagcacgtggcgcagatcctggactcgcgcatgaacaccaagtacgacgagaacgacaagctcatccgtgaggtgaaggtcatcacccttaagtctaagctggtcagtgacttccgcaaggacttccagttctacaaggtccgggagatcaacaactaccaccacgcgcacgacgcctacctcaacgcggtggtggggacggcgcttattaagaaatatcccaagctggaaagcgagttcgtttacggcgactacaaggtgtacgacgtccgcaagatgatcgcaaagtcggaacaggaaatcggaaaggcgacggccaaatatttcttttactccaacatcatgaatttttttaagacggagatcaccctggcgaacggggagatccgcaagcggcccctcatcgagaccaacggggagacgggcgagatcgtctgggacaagggccgggacttcgccaccgtgcggaaggtgctttctatgcctcaagtcaatatcgtcaaaaagacagaggtgcagaccggcgggttcagcaaggagtctatcctgccgaagcgcaactcggacaagctcatcgcgcgcaagaaagactgggaccccaaaaaatatggcgggttcgactcgccgaccgtcgcctacagcgtcctcgtggtggctaaggtcgagaagggcaagagcaaaaagctaaagtcggtgaaggagctgctgggcatcaccatcatggagcgctcgtctttcgagaagaatccaatcgacttcctagaggcgaaggggtacaaggaggtcaaaaaggatcttatcatcaaactgccgaagtacagtctgttcgagctggagaacgggcggaagcggatgctggctagtgcgggcgagttgcagaagggcaacgagttggcactgccctccaagtacgtgaacttcctgtacctggcctcccactacgagaagctcaaggggagccccgaggacaacgagcagaagcagctattcgtcgagcagcacaagcactacctggacgagatcatcgagcagatcagtgagttctccaagcgggtcatcctcgcggacgccaacctggacaaggtgctgagcgcgtacaacaagcacagggacaagccaatcagggaacaggccgagaacatcatccacctgttcaccctgaccaacctgggtgcaccggctgccttcaagtactttgacacgaccatcgaccggaagcgctacacctccacgaaggaggtgctggacgccacgctgatccaccagagcatcaccgggctctacgagacacggatcgacctgagccagcttggcggggac

SEQ ID NO: 66 - DNA, 4092 bp, synthetic construct
  misc_feature 1..4092: note = Synthetic polynucleotide
  source 1..4092: mol_type = other DNA
gacaaaaagtattccattggactcgctatcggcacgaacagcgtcgggtgggcggtcatcactgacgagtacaaggtgccgagcaagaagtttaaggtgctgggaaacaccgacaggcactcgatcaagaaaaatcttatcggggccctactcttcgactccggagaaaccgccgaggccacccggttgaagcgcacggcccgccgtcgctacaccaggcgcaagaaccggatctgctacctccaggagatattcagcaatgagatggcgaaggtggacgactcgttttttcacaggctagaggagtctttcctcgtggaggaggacaagaaacacgagcgccaccccatcttcggcaacatcgtggatgaggtggcatatcacgagaagtacccaaccatctaccacctccgcaaaaagctcgtggactctaccgacaaggccgacctccgtctgatctacctcgcgctggcccacatgattaagttccgaggacactttctgatcgagggcgacctgaacccagacaacagcgacgtggacaagctgttcatccaacttgtccagacctacaatcagctcttcgaggagaaccctatcaacgcctcgggcgtggacgcgaaggccatcctgtccgcccgcctgagcaagtcgcggcggctggagaacctgatcgcccagctccccggcgaaaaaaagaacggcctcttcggcaacctcatcgcgttgtcgctggggctcaccccgaacttcaagtccaacttcgacctggccgaggacgctaaactccagctctcgaaggatacctacgacgacgacctcgacaacctgctggcccagatcggcgaccagtacgcggaccttttcctggcggccaagaacctgagcgacgcgatcctccttagcgacatactccgtgtgaacaccgagatcacgaaggccccgctctccgcgtccatgattaagcgctacgacgagcaccaccaagaccttaccctgcttaaggcgctggtcaggcagcagttaccggagaagtacaaggagatcttttttgatcaatctaagaacggttacgccgggtacatcgacggcggcgcgtcccaggaggagttctacaagttcatcaagccgatcttggagaaaatggacgggaccgaggagctgctcgtgaagctcaaccgcgaagacctcctccgcaagcagcgcaccttcgacaacgggagcatcccgcaccagatccacctgggagagctgcacgcgatcctgcggagacaagaggacttctaccccttcctcaaggacaaccgggagaagattgaaaaaatacttacttttcgtatcccgtactacgtcgggccccttgcgaggggcaactccagattcgcgtggatgacccgcaagtccgaggagaccatcaccccgtggaacttcgaggaggtggtggacaagggcgcgtcggcccagtcgttcatcgagcgcatgaccaacttcgacaagaaccttccgaacgagaaggtgctcccgaagcacagcctgctctacgaatattttactgtgtacaacgagctgacgaaggtcaagtacgttacggaggggatgaggaagcccgccttcctctccggcgagcagaagaaagccattgtggatctcctgttcaagaccaaccgcaaggtgacggtgaaacagctcaaagaggactacttcaagaagatcgagtgcttcgactccgtagagatcagcggggtcgaggaccgcttcaacgcctcgctgggcacgtaccacgacctgctaaagattatcaaggacaaagacttcctagacaatgaggagaacgaggacattctggaggacatcgtgctgactctgacgctgttcgaagaccgcgagatgatcgaggagcggcttaagacgtacgcccacctgttcgacgacaaggtgatgaagcagttgaaacggcggcgctacaccgggtggggccgcctctcccgcaagctcatcaacggcatccgcgacaagcagtcggggaagacgatcctggacttcctcaagagcgacggcttcgccaaccgaaacttcatgcagctaatccacgacgacagcctgacgttcaaggaggacatccagaaggcccaagtgagcggccagggagactcgctacacgagcatatcgccaacctggctggcagcccggcgattaagaaaggaatcctccaaaccgtcaaagtggtggacgagctggtgaaggtgatgggccgccacaagcccgagaacattgtgatcgagatggcgcgggagaaccagacgacgcagaagggccaaaaaaatagcagggaaaggatgaagcgaatagaggaggggatcaaggagctggggagccagattctcaaagagcacccggtcgagaacacacagctccagaacgagaagctgtacctctactacctccaaaacggccgcgatatgtacgtggaccaggaactagacatcaaccggctgagcgactatgacgtggaccacatcgtgccgcagtccttcctcaaggacgactcgattgacaacaaagtgctcactagatccgacaagaacagaggcaagagcgataacgtcccgtcggaggaggtcgtcaagaaaatgaaaaactactggcggcagctcctaaacgccaagctcatcacgcagcgtaagttcgacaacctgacgaaggcggagcggggcgggctgagcgagctggacaaagcggggttcatcaagcggcagctcgttgagacgcggcagatcacaaagcacgtcgcgcaaatcctcgactcccgcatgaacaccaagtacgacgagaacgacaagctcatccgggaggtgaaggtcattacccttaaatcgaagctcgtcagcgactttcgtaaggacttccagttctacaaggtcagagagatcaacaactaccaccacgcccacgacgcctatctgaacgccgtggtgggcaccgcgcttattaagaagtaccccaagctggagtccgagttcgtgtacggcgactacaaggtttatgacgtcaggaagatgatcgccaagtcggaacaggagatcggaaaagctaccgccaaatatttcttctatagcaacatcatgaacttcttcaaaaccgagatcaccctcgccaacggcgagatccggaagcgcccgctcatcgagaccaacggggagaccggggagatcgtctgggacaaggggcgggacttcgctactgtccgaaaggtgctctccatgccacaagtgaatatcgtcaagaaaacagaggtgcagaccggagggttcagtaaggagtccatcctgcccaagcggaactccgacaagctaattgctcgcaaaaaggattgggatcctaaaaaatatggcggcttcgactcgcccacggtcgcctactctgtgctggtcgtggcgaaggtggagaagggcaagtccaagaagctcaagagcgtcaaggagctgctggggatcacgatcatggagcgtagttcgtttgagaagaatcccatcgacttcctggaggctaagggctacaaggaggtcaaaaaggacctcatcattaagctgccgaagtacagcctcttcgagctggagaacgggcggaagcgtatgctcgcctccgctggggagttacaaaaggggaacgagctggcgctgccgtctaagtacgtcaacttcctgtacctggcctcccactacgagaagctcaaggggtcgccggaggacaacgagcagaagcagctcttcgtagagcagcacaagcactacctggacgagatcatcgagcagatttcagagttctcaaagcgggtcatcctcgccgacgccaacctggacaaggtgctctcggcctacaacaagcaccgggacaagccgatccgcgaacaggccgaaaacatcatccacctgttcacgctcaccaacctcggtgccccggcggccttcaagtactttgacacgaccatcgaccggaagcgctatacctcgacgaaggaggtgctggacgccaccctgatccaccagtccatcaccgggctttacgagacccggatcgacctctcgcagcta

SEQ ID NO: 67 - DNA, 4101 bp, synthetic construct
  misc_feature 1..4101: note = Synthetic polynucleotide
  source 1..4101: mol_type = other DNA
gacaagaagtatagtattggactcgccatcggaaccaactctgtggggtgggctgttattacagatgaatataaggtgccatccaaaaagtttaaagttctgggcaatactgatagacactcaatcaagaagaatctgataggtgcacttctgtttgatagtggagagactgccgaggcaaccagacttaaaaggactgcaagaagaagatataccagaagaaagaataggatttgctatttgcaggaaatcttcagcaacgaaatggccaaggttgatgactcatttttccataggttggaggagagttttcttgtggaggaagataagaagcacgaaagacacccaattttcgggaatatagtggacgaggtggcttatcatgagaagtatcccactatctaccacctgagaaagaaacttgtggactcaaccgataaggctgatcttaggcttatatacttggcccttgcacatatgatcaaattcaggggccattttcttatcgaaggcgatcttaatcccgataactcagatgtggacaagctgtttatacaacttgtgcaaacctacaatcaactcttcgaggagaatcccattaacgcctccggcgtggatgcaaaagccatactgtcagccagactgagcaaaagtaggagactggagaatcttatagcccaactgcccggtgaaaagaagaatgggctcttcggaaatctgatcgctctttcattggggttgacacccaactttaagagtaactttgacttggcagaagatgcaaagttgcagctcagtaaagacacatatgacgatgaccttgacaatctcttggcacaaataggggatcaatacgctgaccttttcctcgctgccaagaacctcagcgacgctatactgttgtccgacattcttagggttaataccgaaattacaaaggcccctcttagtgcaagtatgatcaaaaggtatgatgagcatcaccaagaccttacactgctgaaggctctggttagacagcaactccctgaaaagtataaggaaatattcttcgaccaaagtaagaacgggtacgccggttatattgatgggggcgcaagtcaagaagaattttacaaattcatcaagccaattcttgaaaagatggacgggactgaggaattgctggtgaaactgaatagagaggaccttcttagaaaacagaggacatttgacaatgggtccatcccacaccagattcatctgggggaactccacgcaatattgaggagacaagaagacttttacccattccttaaggataatagagagaaaatcgaaaaaatcctgactttcaggattccttactatgttgggccactggccagggggaactcaagattcgcttggatgacaaggaagtcagaagaaaccataaccccttggaattttgaagaggtggttgataagggggcatcagcccagtctttcatagagaggatgaccaactttgataaaaatcttccaaatgagaaggttttgccaaaacatagtcttttgtacgagtactttactgtttataacgaattgaccaaggtgaagtatgtgaccgagggaatgaggaagccagcatttttgtccggggagcaaaagaaagcaatcgttgatcttctcttcaagaccaacagaaaagtgaccgtgaaacaactgaaggaagactacttcaaaaagatagaatgtttcgattcagtggaaattagcggtgttgaagacaggttcaatgcttcattgggtacttaccacgacctgttgaagataatcaaagacaaggactttctcgataatgaggagaacgaagacatcttggaagacattgtgcttacactcactttgtttgaggacagggaaatgattgaggaaagactcaaaacttacgctcatttgtttgatgataaggttatgaaacaactaaaaagaagaaggtacaccggctggggaagattgagtaggaaactgatcaacggtattagagataaacaatccggaaagactatcctcgatttccttaagagtgatggctttgcaaataggaattttatgcagctgattcatgacgactcacttaccttcaaagaagacatccaaaaagctcaggtgtctgggcaaggcgacagtctgcatgaacatatagctaacttggctgggagtcccgccatcaagaaggggatacttcaaacagttaaagttgtggacgaattggtgaaggtaatgggaaggcacaagcctgaaaatatagtgatagaaatggcaagggaaaatcaaacaacccagaagggacagaagaacagtagggaaaggatgaaaaggatagaagaggggatcaaagagcttggtagccagatcctcaaggaacatccagtggagaatacccaacttcaaaacgagaaactctatttgtactacttgcagaacggaagagatatgtatgtggaccaagagcttgatattaacaggctgagcgattatgacgttgaccacatagtgccccaatcattcctcaaggatgactctattgataataaggtgctgacaaggagtgacaagaatagagggaaatccgacaacgttccatccgaggaagttgtgaagaagatgaagaactactggaggcagttgctgaacgctaagctcattacccagaggaaattcgataacctgaccaaagcagagagaggcgggctgagcgaactcgataaagcaggtttcatcaagagacaactcgtggagactaggcaaattactaagcacgtggctcaaatactcgacagcaggatgaacacaaagtacgacgagaacgacaagctcattagagaggttaaggttattactctgaaaagtaaattggttagcgatttcagaaaggatttccaattctataaggttagagagatcaacaattatcatcatgcacatgatgcctatctgaatgctgtggttggtacagcccttatcaagaagtaccctaagctagagagcgagtttgtgtacggagattataaggtgtatgatgtgaggaaaatgatcgctaaaagtgagcaagagattggaaaggctaccgccaaatacttcttttattccaatattatgaatttcttcaagacagaaatcaccctggctaacggcgagataaggaagaggccgcttatcgaaactaatggggagacaggcgaaatagtgtgggacaaagggagggatttcgcaactgtgaggaaggttttgagcatgcctcaggtgaatatcgttaagaaaaccgaagttcaaactggagggttctctaaggaaagcattctccccaagaggaactccgacaagctgattgctagaaagaaagactgggaccccaagaagtatggcggattcgactcacccactgtggcatatagcgttctcgtggtggcaaaggttgaaaagggtaaatccaaaaaactcaaatccgtgaaggaactccttggcataactattatggaaaggagtagctttgaaaagaatcccatcgactttctcgaagctaagggctataaggaagttaagaaggaccttataatcaaacttccaaaatactccctttttgagttggaaaacggcagaaagagaatgttggccagtgccggggagcttcaaaagggcaacgaactggctctgcctagcaaatatgtgaactttttgtatctggcatcacactacgagaaacttaaaggctctcctgaggacaacgagcaaaaacagctctttgttgaacagcataagcactacctcgacgagattattgagcagatcagcgagttctcaaagagagttattctggctgacgctaatcttgacaaggttttgtccgcttacaacaaacacagggataagccaatcagggagcaggcagaaaacataatccatctctttaccctgacaaacctcggtgcccccgctgctttcaagtattttgatactaccattgacaggaagagatatacttccactaaggaagtgctcgacgcaaccctcatacaccaaagtatcacaggcctctatgaaactaggatagatttgtctcaacttgggggcgat

SEQ ID NO: 68 - DNA, 4101 bp, synthetic construct
  misc_feature 1..4101: note = Synthetic polynucleotide
  source 1..4101: mol_type = other DNA
gacaaaaagtattccatcgggcttgctatcggaaccaactctgtggggtgggcagttattaccgacgaatacaaggtgcccagcaagaagtttaaggttctggggaacacagatagacatagcataaagaaaaacctgataggcgcactgttgttcgactccggggaaacagccgaagctaccaggctgaagagaactgcaagaagaaggtacaccagaagaaaaaacagaatatgttatctccaagagattttctctaacgagatggccaaggtggacgactcattctttcacagactggaagaatctttccttgtggaagaagataagaaacacgagaggcaccctatttttggcaatatcgtggatgaggtggcttaccacgaaaaataccctacaatataccacctcaggaaaaaattggttgatagtacagacaaggccgacctcaggctcatctatttggccctggcccatatgattaaattcagggggcactttctcatcgagggagatttgaaccccgacaacagtgatgttgataagctctttattcagctcgtgcagacttacaatcagttgtttgaggaaaaccccattaatgcttccggggtggacgccaaggcaatcctttctgcaagactctcaaagtcaaggagactcgaaaatctgatagcacagcttccaggagagaagaagaacgggctctttggaaacctgatcgctctgtcactcggactcacacccaatttcaaaagcaattttgatttggcagaggacgctaagctgcaactcagtaaggatacctacgacgatgacttggataatctgctcgcacaaattggggaccagtatgcagacctgtttctcgcagctaagaacttgagtgacgccatattgctcagtgacatcctcagggttaataccgagattacaaaagctccactctctgcaagcatgatcaagaggtatgacgagcaccatcaagacctgacactccttaaggcgttggttaggcagcaacttcctgaaaagtataaggaaatcttcttcgatcaaagcaaaaacggctacgccggctatatagacgggggagcatcccaagaagaattttataagttcataaaacctatattggagaagatggacgggacagaggaattgctcgtgaaactgaacagggaggatctcctcaggaagcaaaggaccttcgacaatggctccatcccacatcagattcacctcggcgaactgcacgcaatactgagaagacaagaggacttttatcctttcctgaaggacaacagggagaaaatcgagaaaatcttgacattcagaatcccatactacgttgggcctctggccagaggtaacagtaggttcgcctggatgactaggaaatcagaggagactattacaccctggaactttgaagaagttgttgataagggagcttcagcacaatcattcatcgaaagaatgacaaactttgacaaaaatctgcctaatgagaaagtgctcccaaaacattccctgctgtatgagtattttaccgtttataacgagcttaccaaggtgaaatacgttactgaaggtatgagaaagccagcttttctttcaggggagcaaaagaaggctatcgtggatcttctctttaagaccaacagaaaggttaccgtgaagcagcttaaggaagactactttaaaaagatcgagtgttttgactcagtggaaataagcggtgttgaagatagattcaacgcatccttgggaacttatcatgatcttcttaagataatcaaggataaagactttctcgacaacgaggaaaacgaagatatactggaggacatagttctgacacttactttgttcgaggatagggagatgatcgaggaaagactgaaaacatatgctcaccttttcgacgacaaagttatgaaacaactcaagagaaggagatatacagggtgggggagattgagcaggaaactgattaatggtatcagagacaaacagtcaggaaaaacaatactcgactttttgaaatcagacgggttcgcaaataggaatttcatgcagcttatacacgacgattcacttacttttaaagaggacattcaaaaggctcaagttagtggacaaggtgactccctccacgaacacatcgcaaatctcgctggcagccctgcaattaagaagggtatactccagacagttaaggttgttgacgagctggttaaagtgatgggaagacacaaacccgagaacatagtgatagagatggccagggaaaaccaaaccactcaaaaagggcagaaaaattccagagagaggatgaaaaggattgaagaaggtatcaaggagctgggtagccaaattctgaaagaacatcctgtggaaaacactcaactccagaatgagaaactctatctgtactatctgcaaaatgggagagatatgtatgtggaccaggaactggacataaacaggctctcagattacgatgtggatcatatcgtgccacagtcctttcttaaggatgatagcatcgacaataaggtgcttaccaggtccgacaagaacaggggaaagtcagataacgtgccttctgaagaagttgttaaaaagatgaagaactactggagacagctgcttaacgctaagctcataacacagaggaagtttgacaacttgaccaaggccgagagaggcggactctcagaattggataaggcagggttcataaaaaggcagctggtggaaacaaggcagataactaaacatgtggctcagatcctcgatagtaggatgaatacaaaatacgatgagaacgacaagctcataagggaggttaaagtgataactctgaaatccaaactggttagcgattttaggaaggatttccagttttacaaagttagggagatcaacaattatcatcacgcccacgatgcctacttgaacgcagttgtgggtactgcacttatcaaaaagtaccctaagctggaatccgagtttgtttatggagactataaggtgtacgacgttagaaaaatgattgcaaagtcagagcaggagatagggaaagccactgcaaaatatttcttttatagcaatatcatgaatttctttaagacagaaatcacactggccaatggggaaataaggaagaggcccctgatcgaaactaatggcgagacaggggagattgtgtgggataaaggtagggactttgcaacagtgaggaaagtgctgagcatgccccaagttaatatcgttaaaaagaccgaggttcaaacagggggctttagtaaggaaagcattttgcccaagaggaatagtgacaaattgattgctaggaaaaaagattgggaccccaaaaagtatggcggatttgatagccccactgttgcttactccgtgctcgtggttgcaaaggtggagaagggaaagagcaagaaactgaagtcagttaaggaactccttggtatcactatcatggaaagaagctcctttgagaagaaccctattgacttcctggaggctaaagggtacaaagaggttaagaaagaccttatcattaaattgcccaaatatagtcttttcgagcttgaaaacggaagaaagaggatgcttgcatccgctggcgaattgcaaaagggcaatgagcttgctctcccttccaagtatgtgaacttcctttatcttgcctcacactatgaaaaactcaaaggttcacccgaagacaacgaacaaaagcaactatttgtggaacaacacaagcactacctggacgaaatcattgagcaaatttctgagttttcaaaaagggtaatcttggctgacgcaaatctcgacaaagttttgtcagcttacaacaaacatagagataagccaattagagagcaagctgagaatatcatccatctgtttaccctgactaaccttggagcgcctgctgcttttaaatatttcgacaccacaatcgacaggaagaggtacactagcactaaggaagttctcgacgccaccctcatccaccagagtattacaggcctgtacgagacaagaattgatctttctcaacttggtggtgac

SEQ ID NO: 69 - DNA, 4101 bp, synthetic construct
  misc_feature 1..4101: note = Synthetic polynucleotide
  source 1..4101: mol_type = other DNA
gataagaagtactcaatcggtctggcaatcggaaccaactctgtgggttgggcagtgattacagatgagtataaggtgccaagcaaaaaattcaaggtgctgggtaataccgacagacacagcattaagaagaatttgattggagcactcctctttgactcaggggaaacagcagaggcaacaaggctgaagaggacagcaaggcggaggtacacaaggcggaaaaacaggatatgctacctccaggaaatctttagcaacgagatggctaaagtggatgatagctttttccatagactcgaagaatcctttcttgttgaagaggacaaaaagcatgaaaggcatcccatcttcggcaatatagttgatgaggttgcataccatgagaagtaccccacaatctaccacctcagaaagaaacttgtggactccacagataaagcagacctgaggctcatatacctcgcactcgcacacatgatcaagttcagagggcactttctcatcgaaggtgacctgaatccagataattcagatgtggataaactgtttatacagctggtgcaaacatacaaccaacttttcgaggaaaacccaatcaatgcctccggtgttgatgcaaaggccatcctgtcagcaagactcagcaaaagcaggcggctcgaaaacctcatcgcccagcttcccggtgaaaagaagaacgggctctttggtaatctcatcgcattgagccttggtcttactccaaacttcaagagcaattttgatctggcagaggatgctaaactgcaactctcaaaggacacatatgacgatgaccttgacaatctgttggcccagatcggggaccaatatgcagacctcttcctggccgcaaagaatctgtcagatgcaatcctcttgtccgacatactgagagttaacactgagatcacaaaggcacctctgtccgcctccatgattaagagatacgatgagcatcaccaggatctgactttgctcaaagccctcgttagacagcagttgccagaaaagtacaaagaaatattctttgatcaatcaaaaaacggatatgcagggtacatcgacggtggggcaagccaggaagagttctacaaattcatcaaacctatcctggaaaagatggatgggacagaagagctgctggttaagctgaatagggaagacctcctcagaaagcagaggacatttgataacgggagcatccctcatcaaatccacctcggtgaactccatgctatcctgagaaggcaggaagacttttatccatttttgaaggacaatagggagaaaatcgaaaaaatcctgacattcagaatcccatactacgttggtcctctggcaagaggtaacagtaggttcgcatggatgacaaggaaaagcgaggagacaatcacaccctggaattttgaggaagttgttgacaagggtgccagcgcacaatcctttatcgaaagaatgacaaatttcgacaagaatctgcctaacgaaaaggttctcccaaagcattcactcctgtacgaatattttacagtttataacgaactgactaaagttaaatacgttaccgagggtatgaggaagccagcattcctttccggggaacagaagaaagctattgtggacctcctgttcaagacaaatagaaaagtgacagttaagcaactcaaagaggattacttcaaaaagatcgaatgttttgactctgtggagatcagcggggtggaggatagattcaacgccagcctgggtacatatcatgatctcctgaaaatcattaaagacaaggacttccttgacaacgaggagaacgaggacattctggaagacattgttctgaccctcacactctttgaggatagggagatgattgaggaaagactgaagacctacgcccacctctttgacgataaagtgatgaaacagctcaagagaagaaggtatacaggttgggggagactgagcaggaagttgatcaatgggattagggacaaacagtccgggaaaacaatcctcgattttctgaagtcagacggtttcgcaaacagaaattttatgcagctcattcacgatgacagcttgacattcaaggaagacatccaaaaggctcaagtgagcggccaaggggatagcctccacgagcatattgcaaatctggcaggttcaccagccatcaaaaagggcatacttcagacagttaaggttgtggacgaattggttaaagttatgggcaggcataagccagagaatatcgttatcgaaatggcaagggagaaccaaacaactcaaaaagggcagaaaaatagcagagagaggatgaaaagaatcgaggaagggatcaaggaacttgggtcccaaatcctcaaggagcacccagttgaaaatactcaactgcaaaacgagaagctctatctctactatctccaaaacgggagggatatgtatgttgaccaggagctggatattaacagactgtcagattatgatgttgatcatatcgtgccccagtcattcctgaaggacgattccatcgacaacaaagttctcacaaggtccgataaaaacaggggcaagtccgataacgttccaagcgaagaagtggtgaaaaagatgaaaaactattggagacaacttctgaatgcaaagttgattactcagagaaagtttgacaacctcacaaaagcagaaagaggcgggcttagcgaactcgataaggcagggtttatcaaaagacagctggttgagacaaggcagatcacaaaacatgtggcacagatccttgactcaaggatgaataccaagtatgatgagaatgataagttgatcagggaggttaaagttatcacactcaaatccaaactggtgtcagacttcaggaaagactttcaattttataaggtgagggagatcaataactaccaccatgcacatgacgcctacctgaacgcagtggtgggtacagcattgattaaaaaataccctaagctggagtctgagtttgtgtacggggactacaaggtgtacgacgtgaggaaaatgatagccaagtccgagcaggagatcgggaaagcaacagctaagtatttcttttacagtaatatcatgaatttctttaaaactgagattactctggcaaacggggagatcaggaaaagacccctcatcgagactaatggtgaaacaggtgagatcgtttgggacaaggggagggattttgctactgttagaaaagttctgagtatgccacaagtgaatattgtgaaaaagacagaagttcagacaggtgggttctccaaagaatccatcctgcccaagagaaattcagacaagctcatcgcaagaaagaaggactgggaccctaagaagtacggaggatttgacagccccaccgtggcctattccgtgcttgttgtggcaaaggtggagaaagggaagagcaaaaaactgaaatccgtgaaagaactgctgggaattaccatcatggaaagaagctcctttgagaagaacccaatcgacttcctggaagcaaaaggatataaggaagtgaaaaaggacctcattatcaagctcccaaaatactcacttttcgagttggagaacggtagaaagaggatgctggcaagcgcaggggaacttcagaaaggcaatgagctggcattgccatcaaagtatgtgaacttcctctacttggccagccattacgagaaacttaaaggtagcccagaagataacgagcaaaaacagctctttgtggaacagcataagcattatctggatgagatcatagaacaaatctcagagttttccaagagagttatcctcgcagatgcaaacctggataaggttctctcagcctataataagcatagagacaagccaattagagagcaagcagagaacattatccacttgttcactcttacaaacctgggggcaccagccgccttcaaatatttcgatacaacaatagacagaaagaggtataccagcaccaaagaagttctcgacgccacactgatccatcaatcaatcacaggcctttacgaaactaggatcgacttgtcacaactgggtggggat

SEQ ID NO: 70 - DNA, 3307 bp, synthetic construct
  misc_feature 1..3307: note = Synthetic polynucleotide
  source 1..3307: mol_type = other DNA
gagcaaggacacctacgacgacgacttggacaacctattggcccagataggtgaccagtatgcagacctcttccttgcggccaagaacttgagtgacgctatactgctcagtgacatcctgagggtgaacactgagatcactaaggcccctctctctgcctcaatgattaagcgttacgacgagcatcaccaggatctcaccctgcttaaggcccttgttcggcagcagctccctgagaagtacaaggagatattttttgaccagtctaagaacggctacgccggttacattgacggtggggcaagccaggaggagttctacaagttcatcaagccgatccttgagaagatggacggcaccgaggagctacttgtcaagttgaaccgggaagacctgctccggaaacagcgtacattcgacaacggcagcatccctcaccagatccacctgggcgaactacacgccatcctccgacgtcaggaggacttctatccattcttgaaagataacagggaaaaaatcgaaaaaatacttacgtttcgaataccttactacgtggggccccttgctcggggaaactccagattcgcatggatgaccaggaagtcagaggagaccatcacaccctggaactttgaggaggtggttgacaaaggtgcttctgcccagtccttcattgagcggatgactaacttcgacaagaacctgcccaacgagaaggtgctgccaaagcacagcctgctctacgaatactttactgtgtacaatgagctgacgaaggtgaagtacgtgacagaggggatgcggaagcccgctttcctgagcggcgagcaaaaaaaagcaatcgtggacctactgttcaagaccaaccgaaaggtgacagtgaagcagctcaaggaggactacttcaaaaaaatcgagtgcttcgactctgttgagataagcggcgtggaggaccgattcaacgcctcattgggaacctatcacgacctgctcaagatcattaaggacaaggacttcctggataatgaggagaatgaggacatcctggaggatattgtgctgacccttactctattcgaggacagggagatgatcgaggagcgactcaagacctacgctcacctgttcgacgacaaggttatgaagcaattgaagcgtaggcgatacacggggtggggaagactctcccgaaaactgataaacggcatcagggacaagcagtcagggaagacgatcttggacttcctgaaatccgacgggttcgccaaccgcaacttcatgcagctcattcacgacgactcactaacgttcaaagaggacattcagaaggctcaagtcagtggacaaggcgactccctgcacgagcacattgcaaaccttgcgggctccccggcgattaaaaagggcattctccaaacggttaaggtggtggacgagctggtgaaggtgatgggccgacacaagcctgagaacatcgtgatcgagatggccagggagaaccagactacccagaagggtcagaagaactctcgggaacgtatgaagcgtattgaggaggggattaaggagttgggctctcaaatcctcaaggagcaccctgtggagaacactcagctccaaaacgagaagctgtacctgtactacctgcaaaacgggcgcgatatgtacgtggatcaggagttggacatcaacaggcttagcgattacgacgtggaccacatcgtgccacagtcattcttaaaggacgacagcatcgacaacaaggttctgacgaggagcgacaagaatcgagggaaaagtgacaatgttccatccgaggaggtggtcaagaaaatgaagaactattggcgtcagcttctgaacgccaagctcatcacccagcggaaattcgacaacctgactaaggctgagcgaggcggactctccgagcttgacaaggctggcttcatcaagcggcagttggtcgaaacccgacagataacgaagcacgttgcccagatacttgactcccgtatgaacaccaagtacgacgagaacgacaagctcatcagggaggtgaaggtcattacccttaagtccaaactcgtcagcgactttcgtaaggacttccagttctacaaggtgcgcgagatcaataactaccaccacgcacacgacgcctacctgaacgcagtggttggaaccgcgttgattaaaaagtaccccaagttggagtcggagttcgtttacggggactacaaggtgtacgacgttcggaagatgatcgccaagtctgaacaggagatcgggaaagcaaccgccaagtatttcttctatagcaacatcatgaacttctttaaaaccgagatcacacttgccaatggcgagatccgtaagaggccgctgatcgagacaaatggggagactggcgagatcgtgtgggacaagggccgcgacttcgcaaccgttcggaaagtcttgtccatgcctcaagtcaacatcgtcaagaagactgaggtgcaaacaggcgggttctcgaaggagtccatactgcccaagaggaactcagacaagctcatagcacgcaaaaaagactgggatccaaagaaatacggcgggttcgactcgccgacagtcgcatactccgtgttagtggtggctaaagtggaaaaggggaagtccaagaagctcaagtccgtcaaggagttgctcgggatcaccattatggaacggtcctcattcgagaagaatcccattgacttcctagaggcgaagggctacaaagaggtcaaaaaggacctaattattaagctccccaagtattcactcttcgaacttgaaaatggtcgtaagcggatgttggcaagcgctggagagcttcagaaggggaacgagcttgcactgccttccaagtacgtgaacttcctgtacctcgcctctcattacgagaagttgaagggctcaccggaggacaacgagcagaagcagttgttcgtggagcagcacaagcactacctcgacgagatcattgagcagataagtgagttcagcaaacgggtgatccttgccgacgctaacctggacaaggtgctgagcgcctacaacaagcacagagacaagccgatccgagagcaagcggagaacatcatacacctgttcaccctcacgaacctcggggctcccgcagccttcaaatattttgacacgaccatcgaccgtaaacgctacactagcacgaaggaggtgctggacgctacccttatccaccagtccatcaccggcctgtacgagacgagaatcgacttgtcgcagctcggtggtgac

SEQ ID NO: 71 - DNA, 4101 bp, synthetic construct
  misc_feature 1..4101: note = Synthetic polynucleotide
  source 1..4101: mol_type = other DNA
gacaaaaaatactcaattggtctggcaattgggaccaacagtgtcggatgggccgtgattaccgacgagtacaaggtgccgtccaaaaaattcaaggtgcttgggaacaccgaccgccactcgatcaagaaaaacctaatcggtgcgttgcttttcgacagtggggagaccgccgaggcaacacgcttaaaacgcacagctaggaggagatatacacggcgcaagaaccgaatatgctacttacaggagatattctccaatgagatggcgaaggtggacgactctttcttccatcggcttgaggaatccttcctggtcgaggaggacaagaagcacgagcgacacccgatattcgggaacatcgttgatgaggtggcgtaccacgagaagtacccaacgatataccacttacgcaagaagctcgtggactctacggacaaggccgacttgcgccttatctacttggcactggcccacatgattaagttccgaggccacttccttatcgagggtgacctgaaccccgataactccgacgtggacaagctcttcatccaactcgtccagacatacaaccagctattcgaggagaatcctatcaacgcctctggggtggacgctaaagctatcctctcagcccgcctgtcaaagtcgaggaggttggagaacctaatcgcccagcttccaggcgagaagaaaaatgggctgttcggaaaccttatcgcactctcactgggcctaaccccgaacttcaagtccaacttcgacctggcagaggacgcgaaattgcagttgtcgaaagacacctatgacgatgacctggacaacctgttggcccagataggggaccagtacgccgacctgttcctagcggccaagaacctgtccgacgccatcttgctgtcggatatactgcgggtgaacaccgagatcactaaagcacctctctccgccagcatgattaagcgttacgacgagcaccaccaagatttgaccctgctaaaggcacttgtacggcagcagcttcccgagaagtacaaggagatctttttcgaccaaagcaagaacggctacgccgggtacatcgacggaggtgccagccaggaggagttctacaagttcattaagcccatcctggagaagatggacgggactgaggaactacttgtgaagctgaaccgggaagacttactacggaagcagcgtaccttcgacaacggttctatcccacatcagatccatcttggggagttgcacgcgatcctgcgacgccaggaggacttttaccccttcctgaaagacaaccgcgagaaaatcgagaagatactgaccttcagaataccttactacgtcggaccccttgcgcgaggcaactcaagattcgcgtggatgaccaggaaatcagaggagaccatcacaccctggaatttcgaggaggtggttgacaagggtgcctccgcccagtcctttatcgaacgaatgaccaacttcgacaagaacttgcccaacgagaaggtgctccccaaacacagcctcctctacgaatatttcacagtgtacaacgagcttactaaagttaagtatgttactgagggcatgaggaaacccgccttcctgtcaggcgagcagaagaaagctattgtggacctccttttcaagaccaaccggaaggtgacagtgaagcagctcaaggaggactacttcaagaagatagagtgcttcgacagcgtggagatcagcggggtggaggacagattcaatgcctctctcggaacataccacgacttgcttaagatcatcaaggacaaggacttcctcgacaacgaggaaaacgaggatattctggaggatattgttctgactcttaccctgttcgaggaccgggagatgatcgaggagcgtctcaagacctacgcccacctgttcgacgacaaagttatgaagcagctcaagcgtcggagatataccggatggggccgtctgtctcggaagctcatcaacgggatcagggacaagcagtcagggaagacgatcttagacttccttaagtctgacggcttcgccaacaggaacttcatgcagttgatccacgacgacagccttaccttcaaggaggacatccagaaggcccaagtgagtggccagggtgacagcctccacgagcatattgctaatcttgcgggttccccagcgattaaaaagggcatacttcaaaccgttaaggtggtggacgagcttgtcaaggtgatggggcgacacaagcccgagaacatcgtgatcgagatggccagggagaaccagaccacccagaaggggcagaagaatagccgagaacgcatgaagcgcatcgaggaggggattaaggagctagggagccagatcctcaaggaacatcccgtcgagaacacccagctccagaacgagaagctatacctctactacttgcaaaacgggagggatatgtacgtggatcaggagttggacattaaccgcctaagcgactacgacgtagatcacatcgtgcctcagtcattcctcaaagacgacagcattgacaacaaagtcttgacccgatccgacaagaaccgaggaaaatccgacaatgtgccctcagaggaggtcgtcaagaaaatgaagaactattggaggcagctacttaacgccaaactcataacccagcggaagttcgacaacctgacaaaggctgagcggggtgggctcagcgagcttgacaaggctggcttcatcaagcggcagttggtggagacaagacagataacgaagcacgtggctcagatcctggactctcgcatgaacacgaagtacgacgagaacgacaaattgatccgcgaggtcaaggttattacgctcaagagcaaacttgtcagcgatttccgcaaggacttccagttctacaaggtgagggagattaacaactaccaccatgcacatgatgcctacttgaacgcagtggtggggaccgcgcttattaaaaagtaccctaagttggagtcagagttcgtttatggggactacaaggtgtacgacgtccggaagatgattgcaaagtctgaacaggaaatcgggaaggccaccgccaaatatttcttctacagtaacattatgaatttttttaagactgaaattactctcgcaaacggcgagatcaggaagcgtcccctcatcgagacaaacggggagaccggggagatagtctgggacaaggggcgggacttcgctacggtgaggaaggtgctctcgatgccacaagtgaacatcgtcaaaaagacagaggtgcagaccggtggcttctcaaaggagtcaatcctgccaaaacgtaacagcgacaagctcatcgcccgcaagaaagactgggaccctaagaagtatggtgggttcgactcaccgacggtcgcatactccgttctggtcgtggcaaaggtggaaaagggcaagtccaaaaaactgaaatccgtgaaggagttgcttggcattaccatcatggaacgcagcagcttcgagaagaaccccattgacttcctggaggctaaagggtacaaggaggtcaagaaagatttaattattaagctacctaagtacagcttgttcgagctggagaacggccgaaaacgaatgctcgcatccgccggggaacttcaaaagggcaacgagcttgcgctgccctccaagtacgtgaacttcctgtacttggcatcccactacgagaaactcaagggtagcccagaggacaacgagcagaagcagctattcgtggagcagcacaagcactacctcgacgagataatcgagcagatcagtgagttcagtaagcgggtgatactcgcggacgccaacttggacaaggtgcttagtgcctacaacaagcaccgtgacaagcccatccgagaacaggctgagaacatcatccaccttttcactctgacaaacctcggtgctcccgccgccttcaaatacttcgacactaccatcgacaggaagcgctacacatctacgaaggaagttcttgacgctacgcttattcatcagtctatcacagggctgtacgagacaaggatcgaccttagccaactcggcggggat

SEQ ID NO: 72 - DNA, 4519 bp, Rubus occidentalis
  source 1..4519: mol_type = genomic DNA
gttatctactaacatgccttatcatttgcactaaatgaatatatgatcgaccacattctttcctaaatccaaattactaagcattatcaatttgctatttagttgtctttgttttctccatgatgatgaggtagtaacattgtcatgtgaatcgaagtttagtgaaatcaaatgatttgattcgtttgtaatcaattaaacaatagttctttcatgtggttataaacaatagttccataattgtaatctcaattaaagttcatctaaattatacctcctttccaattaagtttacattgctaagtttttttttttataatatatatatatatatatatatattacagtgcatgattctatttgattgataaaaaatatatttataaagataaaattataattgtaaggtaattcattgataagatatttatatgtaaagctattaattataattgtaagtataattaattgataagttttaaagttgagtaagcaaagtcattgaatctaagatgagtttaatagactcttgcctaaaatgagtaatttcttaatgatgtgtatatgatgtggcatgctatatatgattgatacctccacatcttaggtcttagagcatcttcaacagttttcctaaaaaatctttattatagggactcaaaagttaaaatttctatattttttctttcgtagttccaacagtttttctatcatattctctataatttattatctacaattaaatatggtattatttttaaatttaaaaagtataatcaaataattaatgatgaaaatgaatatatttggaaagaggaaagagaagagggtaaaattttccaaaaagatgtttctctataattttagagtttgctataaatatagagatttttgttttctctctcatcactatctctaaaatagagataggtatagggaagctattggagtaaaaaaatacacaatttttcctaaactagagaaaaaataaattatagggaagctgttggagatgctcttaatgaagttaacaatctttttccattcagcatacatgtcataccttatagcctcatttcacatatatttcacccaaaacttacctccttaattacctctttgggtagtgctagaaggcctcaattaaggcctaacttttgaggtctcaattaagacgtaacttttgagacctcaatcacatgcatgtggcggttcatgtcatattcatgtcatcattttctatcgcatcatattttgcaataactatttataatttttttttaattagattgtactcatatatttatcttaaaatacacaaaaaaaaaattcctagaaaactggtgtatttatgtcaatatatgcaaatgctatatcatttttggtgtttttagataattgttaaagtgttgtttcaaaaatggtgtaccaaacgtatttttgctactagaaaactatgtcatttttgaaaacaaagaactgtatttgtgaagatatcaagcgcaaatttttttttttcctttttgaaaaccaaccacgtactaagagtcatgcaagagtcaacctgatcaatggttcgagttgataattttataacattctgtccataaagaagagaacattttcatctagaaaaagcattgaaccccttgcatcgatgtgacatgcacagagagaaagaaaggatgggaggtgggggactaacaatgttttaatttctaatgtccatgatattctattcatttatactcttcttcttattgctctctttcaagtttcaacacacttagagatctaaaccaagtactggttagcgaacttgaaattactataaatatagagcaatacagggaaaggcaattataagaagtaccaattcttcctgagttttgagttttcttatccatctttgattcacctttacgactccctctctctctctctctctctctctctctctctctctctctctctctctctctctcttgtaagtcccaataatggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgagatcattcttcactctggtgatgacagacccagatgctcctggccctagtgatccttatttgaaggagcacctgcactgggttgtgacagacattccaggcaccacagatgctacatttggaagagaggtggtgagctatgagatgccaaggcccaacatcggcatccacaggtttgtgtttgttctcttcaggcagaaacgaaggcagtcagtgaaccctccttcctcaagggatcacttcaacacccgaagcttcgcagcagaaaatgacctcggtcttcctgtcgctgccgtttacttcaatgcgcagagagaaacagcagcgagaagacgctagctagctagcagcaggaggcaggccagacccccaaaaattccaggctataaccataatatctaattaaataagagaaaagggtggttaatgatttaatcctccctattgttaggttattaggctaaaagtacttaatttcccaaagagagctgtgagtgtttgtgtctctcatttgatttgtgctctctccatcatcgaatggttataagtttgttctgatttctgatcagttcaatgtatctgtaaagtatctatattcgtatattctatggggttcttgtttttggttttgaatctttgatgatcaactagaactgtatagggacacgtggaacaaacccattatctctctttcgcactcttcttctagtttaagtttagggttttggtttccatttcatttcaaccggaaaatcagaaggaatcaacactatgcaccaatcaagtctgcatgcgggccccacactgtcatgtccgatcttaaataactaattaaccgttggtattgtatgttcaaaagtaagtggaaaaatcactgcttaattattaattaaaaatgttatctatgcactttagtagaacgaaaaaataaattaaaaatgaaacacaacatcattttgacatgacacaagcgtgatatttacattacaattaaaataataaattcatattatgtcgatgcctttttacatttctcgttcacaaattatttttttttttgaggaaaatataaactttattaattaatataaaaggattacaatccttagcatgagcatcttaagaaagagcattatagaccacacaaatgacctaatcgatgagtctaaacctagaactctaatcgatgattcaaaacctagaacaatacatacaccgtgcattactgctatcatacaaagtgtttatgtgaacggcattgactatatgttatctacgggatgattacgattttcatttctaaatgccttaatagttttgtttgacaaactagaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacattttcttttaaaattcgttaacatgtagaaagaaataccactaaattgatgcttagttagtatacaatttctagttccacttagttagattatagttattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggaagggaagagagagctgggttcgtgaggtggctatcggagatggaggtgcagcggtgataagctaggctcaggaggagtagcggagagcagtgatgggattgcaggtgaggctcgagcacgggctcgaggacgacgc

SEQ ID NO: 73 - DNA, 519 bp, Rubus occidentalis
  source 1..519: mol_type = other DNA
atggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgagatcattcttcactctggtgatgacagacccagatgctcctggccctagtgatccttatttgaaggagcacctgcactgggttgtgacagacattccaggcaccacagatgctacatttggaagagaggtggtgagctatgagatgccaaggcccaacatcggcatccacaggtttgtgtttgttctcttcaggcagaaacgaaggcagtcagtgaaccctccttcctcaagggatcacttcaacacccgaagcttcgcagcagaaaatgacctcggtcttcctgtcgctgccgtttacttcaatgcgcagagagaaacagcagcgagaagacgctag

SEQ ID NO: 74 - protein, 172 aa, Rubus occidentalis
MARMSEPLVVGRVIGDVLDSFTPTTKMIVTYTTRHVSNGYELLPSAVTTKPRVEIQGGDMRSFFTLVMTDPDAPGPSDPYLKEHLHWVVTDIPGTTDATFGREVVSYEMPRPNIGIHRFVFVLFRQKRRQSVNPPSSRDHFNTRSFAAENDLGLPVAAVYFNAQRETAARRR

SEQ ID NO: 75 - DNA, 549 bp, Rubus occidentalis
  source 1..549: mol_type = genomic DNA
tccatctttgattcacctttacgactccctctctctctctctctctctctctctctctctctctctctctctctctctctctcttgtaagtcccaataatggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgagatcattcttcactctggtgatgacagacccagatgctcctggccctagtgatccttatttgaaggagcacctgcactgggttgtgacagacattccaggcaccacagatgctacatttggaagagaggtggtgagctatgagatgccaaggcccaacatcggcatccacaggtttgtgtttgttctcttcaggcagaaacgaaggcagtcagtgaaccctccttcctcaagggatcacttcaacacccgaagcttcgcagcagaaaatg

SEQ ID NO: 76 - DNA, 220 bp, Rubus occidentalis
  source 1..220: mol_type = genomic DNA
tccatctttgattcacctttacgactccctctctctctctctctctctctctctctctctctctctctctctctctctctctcttgtaagtcccaataatggcaagaatgtcggaacccttggttgttggaagggtaattggagatgttcttgattccttcaccccaactaccaaaatgattgtcacttacactacgaggcacgtctctaatggatatga

SEQ ID NO: 77 - DNA, 140 bp, Rubus occidentalis
  source 1..140: mol_type = genomic DNA
ctctctctctctctctctctctctctctctctctctctctctcttgtaagtcccaataatggcaagaatgtcggaacccttggttgttggaagggtaattggagatgttcttgattccttcaccccaactaccaaaatga

SEQ ID NO: 78 - DNA, 100 bp, Rubus occidentalis
  source 1..100: mol_type = genomic DNA
ctctctctctctctctctctctcttgtaagtcccaataatggcaagaatgtcggaacccttggttgttggaagggtaattggagatgttcttgattcctt

SEQ ID NO: 79 - DNA, 60 bp, Rubus occidentalis
  source 1..60: mol_type = genomic DNA
ctcttgtaagtcccaataatggcaagaatgtcggaacccttggttgttggaagggtaatt

SEQ ID NO: 80 - DNA, 223 bp, Rubus occidentalis
  source 1..223: mol_type = genomic DNA
tctctctctctctctctctctctctctctctctcttgtaagtcccaataatggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggt

SEQ ID NO: 81 - DNA, 143 bp, Rubus occidentalis
  source 1..143: mol_type = genomic DNA
gtcccaataatggcaagaatgtcggaacccttggttgttggaagggtaattggagatgttcttgattccttcaccccaactaccaaaatgattgtcacttacactacgaggcacgtctctaatggatatgagctcttaccttc

SEQ ID NO: 82 - DNA, 103 bp, Rubus occidentalis
  source 1..103: mol_type = genomic DNA
gtcggaacccttggttgttggaagggtaattggagatgttcttgattccttcaccccaactaccaaaatgattgtcacttacactacgaggcacgtctctaat

SEQ ID NO: 83 - DNA, 63 bp, Rubus occidentalis
  source 1..63: mol_type = genomic DNA
gaagggtaattggagatgttcttgattccttcaccccaactaccaaaatgattgtcacttaca

SEQ ID NO: 84 - DNA, 223 bp, Rubus occidentalis
  source 1..223: mol_type = genomic DNA
ctctctctctctctctctctctctcttgtaagtcccaataatggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgaga

SEQ ID NO: 85 - DNA, 143 bp, Rubus occidentalis
  source 1..143: mol_type = genomic DNA
atggcaagaatgtcggaacccttggttgttggaagggtaattggagatgttcttgattccttcaccccaactaccaaaatgattgtcacttacactacgaggcacgtctctaatggatatgagctcttaccttctgcagtcac

SEQ ID NO: 86 - DNA, 103 bp, Rubus occidentalis
  source 1..103: mol_type = genomic DNA
cttggttgttggaagggtaattggagatgttcttgattccttcaccccaactaccaaaatgattgtcacttacactacgaggcacgtctctaatggatatgag

SEQ ID NO: 87 - DNA, 63 bp, Rubus occidentalis
  source 1..63: mol_type = genomic DNA
ttggagatgttcttgattccttcaccccaactaccaaaatgattgtcacttacactacgaggc

SEQ ID NO: 88 - DNA, 224 bp, Rubus occidentalis
  source 1..224: mol_type = genomic DNA
aagtcccaataatggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgagatcattcttcactctggtgatgacagaccca

SEQ ID NO: 89 - DNA, 143 bp, Rubus occidentalis
  source 1..143: mol_type = genomic DNA
ggaagggtaattggagatgttcttgattccttcaccccaactaccaaaatgattgtcacttacactacgaggcacgtctctaatggatatgagctcttaccttctgcagtcaccaccaaacctagagttgagattcaaggcgg

SEQ ID NO: 90 - DNA, 103 bp, Rubus occidentalis
  source 1..103: mol_type = genomic DNA
tcttgattccttcaccccaactaccaaaatgattgtcacttacactacgaggcacgtctctaatggatatgagctcttaccttctgcagtcaccaccaaacct

SEQ ID NO: 91 - DNA, 63 bp, Rubus occidentalis
  source 1..63: mol_type = genomic DNA
ctaccaaaatgattgtcacttacactacgaggcacgtctctaatggatatgagctcttacctt

SEQ ID NO: 92 - DNA, 223 bp, Rubus occidentalis
  source 1..223: mol_type = genomic DNA
ggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgagatcattcttcactctggtgatgacagacccagatgctcctggccctagtgatccttatttgaag

SEQ ID NO: 93 - DNA, 143 bp, Rubus occidentalis
  source 1..143: mol_type = genomic DNA
accccaactaccaaaatgattgtcacttacactacgaggcacgtctctaatggatatgagctcttaccttctgcagtcaccaccaaacctagagttgagattcaaggcggtgacatgagatcattcttcactctggtgatgac

SEQ ID NO: 94 - DNA, 96 bp, Rubus occidentalis
  source 1..96: mol_type = genomic DNA
tgtcacttacactacgaggcacgtctctaatggatatgagctcttaccttctgcagtcaccaccaaacctagagttgagattcaaggcggtgacat

SEQ ID NO: 95 - DNA, 56 bp, Rubus occidentalis
  source 1..56: mol_type = genomic DNA
acgtctctaatggatatgagctcttaccttctgcagtcaccaccaaacctagagtt

SEQ ID NO: 96 - DNA, 223 bp, Rubus occidentalis
  source 1..223: mol_type = genomic DNA
tgagctcttaccttctgcagtcaccaccaaacctagagttgagattcaaggcggtgacatgagatcattcttcactctggtgatgacagacccagatgctcctggccctagtgatccttatttgaaggagcacctgcactgggttgtgacagacattccaggcaccacagatgctacatttggaagagaggtggtgagctatgagatgccaaggcccaacatc

SEQ ID NO: 97 - DNA, 143 bp, Rubus occidentalis
  source 1..143: mol_type = genomic DNA
gagattcaaggcggtgacatgagatcattcttcactctggtgatgacagacccagatgctcctggccctagtgatccttatttgaaggagcacctgcactgggttgtgacagacattccaggcaccacagatgctacatttgg

SEQ ID NO: 98 - DNA, 103 bp, Rubus occidentalis
  source 1..103: mol_type = genomic DNA
gagatcattcttcactctggtgatgacagacccagatgctcctggccctagtgatccttatttgaaggagcacctgcactgggttgtgacagacattccaggc

SEQ ID NO: 99 - DNA, 63 bp, Rubus occidentalis
  source 1..63: mol_type = genomic DNA
tgatgacagacccagatgctcctggccctagtgatccttatttgaaggagcacctgcactggg

SEQ ID NO: 100 - DNA, 220 bp, Rubus occidentalis
  source 1..220: mol_type = genomic DNA
gatccttatttgaaggagcacctgcactgggttgtgacagacattccaggcaccacagatgctacatttggaagagaggtggtgagctatgagatgccaaggcccaacatcggcatccacaggtttgtgtttgttctcttcaggcagaaacgaaggcagtcagtgaaccctccttcctcaagggatcacttcaacacccgaagcttcgcagcagaaaatg

SEQ ID NO: 101 - DNA, 140 bp, Rubus occidentalis
  source 1..140: mol_type = genomic DNA
acattccaggcaccacagatgctacatttggaagagaggtggtgagctatgagatgccaaggcccaacatcggcatccacaggtttgtgtttgttctcttcaggcagaaacgaaggcagtcagtgaaccctccttcctca

SEQ ID NO: 102 - DNA, 100 bp, Rubus occidentalis
  source 1..100: mol_type = genomic DNA
gctacatttggaagagaggtggtgagctatgagatgccaaggcccaacatcggcatccacaggtttgtgtttgttctcttcaggcagaaacgaaggcagt

SEQ ID NO: 103 - DNA, 60 bp, Rubus occidentalis
  source 1..60: mol_type = genomic DNA
ggtgagctatgagatgccaaggcccaacatcggcatccacaggtttgtgtttgttctctt

SEQ ID NO: 104 - DNA, 204 bp, Rubus occidentalis
  source 1..204: mol_type = genomic DNA
aaacctagagttgagattcaaggcggtgacatgagatcattcttcactctggtgatgacagacccagatgctcctggccctagtgatccttatttgaaggagcacctgcactgggttgtgacagacattccaggcaccacagatgctacatttggaagagaggtggtgagctatgagatgccaaggcccaacatcggcatccac

SEQ ID NO: 105 - DNA, 132 bp, Rubus occidentalis
  source 1..132: mol_type = genomic DNA
cctggccctagtgatccttatttgaaggagcacctgcactgggttgtgacagacattccaggcaccacagatgctacatttggaagagaggtggtgagctatgagatgccaaggcccaacatcggcatccac

SEQ ID NO: 106 - DNA, 724 bp, Pyrus pyrifolia
  source 1..724: mol_type = genomic DNA
gaaaaatataaggagtgctattagctcctcctgaattgacttatccattaatcttttctttttacactctctcttctctctctctctctctctctctctctctctcttaaaatgaaaaaagcctcggagcctccggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatattggcatccacaggtttgtgtttgttctcttcaagcagaatcgaagacaatcaatcaacacaccttcctcgagggatcacttcagcactcgaagcttcgcggctgaaaatgacctgggtcttcctgtcgctgccgtctacttcaacgcgcagagagaaactgcagctagaagacgctagctagtagctgtacccagaactcctccatccattatccatatatatgttaaataaaggcttctttatagataggctattgtaactcttgtttccc

SEQ ID NO: 107 - DNA, 519 bp, Pyrus pyrifolia
  source 1..519: mol_type = other DNA
atgaaaaaagcctcggagcctccggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatattggcatccacaggtttgtgtttgttctcttcaagcagaatcgaagacaatcaatcaacacaccttcctcgagggatcacttcagcactcgaagcttcgcggctgaaaatgacctgggtcttcctgtcgctgccgtctacttcaacgcgcagagagaaactgcagctagaagacgctag

SEQ ID NO: 108 - protein, 172 aa, Pyrus pyrifolia
MKKASEPPVVGRVIGDVLDSFTATTKMSVTYNTKLVCNGLELFPSVVTAKPRVEIQGGDMRSFFTLVMTDPDFPGPSDPYLREHLHWIVTDIPGTTDATFGREVVSYEMPKPNIGIHRFVFVLFKQNRRQSINTPSSRDHFSTRSFAAENDLGLPVAAVYFNAQRETAARRR

SEQ ID NO: 109 - DNA, 401 bp, Pyrus pyrifolia
  source 1..401: mol_type = genomic DNA
cttcactgcaacaacaaaaatgtctgtcacttacaacaccaagctagtctgcaatggacttgagctctttccttctgttgtcacagccaaacctagagttgagattcaaggaggggatatgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatattggcatccacaggtttgtgtttgttctcttcaagcagaatcgaagacaatcaatcaacacaccttcctcgagggatcacttcagcactcgaagcttcgcggctgaaaatgacctgggtc

SEQ ID NO: 110 - DNA, 321 bp, Pyrus pyrifolia
  source 1..321: mol_type = genomic DNA
aagctagtctgcaatggacttgagctctttccttctgttgtcacagccaaacctagagttgagattcaaggaggggatatgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatattggcatccacaggtttgtgtttgttctcttcaagcagaatcgaagacaatcaatcaacacaccttcctcgagggatcac

SEQ ID NO: 111 - DNA, 243 bp, Pyrus pyrifolia
  source 1..243: mol_type = genomic DNA
gtcacagccaaacctagagttgagattcaaggaggggatatgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatattggcatccacaggtttgtgtttgttctcttcaagcagaat

SEQ ID NO: 112 - DNA, 165 bp, Pyrus pyrifolia
  source 1..165: mol_type = genomic DNA
atgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatatt

SEQ ID NO: 113 - DNA, 134 bp, Pyrus pyrifolia
  source 1..134: mol_type = genomic DNA
cctggccctagtgatccttatctaagggagcacctgcactggattgtgacagacattccaggcaccacagatgccacatttggaagagaggtggtgagttatgagatgccgaagcccaatattggcatccacag

SEQ ID NO: 114 - DNA, 887 bp, Prunus serotina
  source 1..887: mol_type = genomic DNA
aagcaatataagaagtacatctcaaattcctcctgagttcttatccattaatctttgatcaatagctacaccttctttctcactcttcctcattctctcctagtcacaaaaaaaatggcaagaatgtctgagcctctggttgttgggagagtgataggagatgttcttgattgtttcaccccaacaacaaaaatgtctgtcacttacaacaccaagctagtctgcaatggatatgagctctatccttctgctgtcaccaacaaacctagagttgagattcaaggaggagatatgagaactttctttactctggtcatgacagacccagatgttcctggccctagtgatccttatttaagggagcacctgcactggattgtgacagatattccaggcaccacagatgccacatttggaagagaggtggtgagctatgagatgccaaggcccaacattggcatccacaggtttgtgtttgttctcttcaagcagaaaagaaggcagtctgtgaaccctccttcctcaagggatcatttcagtgctcgaagcttcgcagccgaaaacgacctgggtcttcctgtcgctgccgtttacttcaattgccagagagaaacagcagctagaagacgctagctagctacccaaaaccccaaaggtcctccataatatgttaattttaaataaagttattatcaagtgtgtttcatcatcctcccctatcgtcagagtagtattaggccaaaactactcacgtgtaacaagaataatttccacgagagagcagtgtgtgtctggctctgtcattttactgctctccaatcggactttatgaactaatttgcagtaagtttgaagtctcatctgagtatctctaaaaaaaaaaaaaa

SEQ ID NO: 115 - DNA, 519 bp, Prunus serotina
  source 1..519: mol_type = other DNA
atggcaagaatgtctgagcctctggttgttgggagagtgataggagatgttcttgattgtttcaccccaacaacaaaaatgtctgtcacttacaacaccaagctagtctgcaatggatatgagctctatccttctgctgtcaccaacaaacctagagttgagattcaaggaggagatatgagaactttctttactctggtcatgacagacccagatgttcctggccctagtgatccttatttaagggagcacctgcactggattgtgacagatattccaggcaccacagatgccacatttggaagagaggtggtgagctatgagatgccaaggcccaacattggcatccacaggtttgtgtttgttctcttcaagcagaaaagaaggcagtctgtgaaccctccttcctcaagggatcatttcagtgctcgaagcttcgcagccgaaaacgacctgggtcttcctgtcgctgccgtttacttcaattgccagagagaaacagcagctagaagacgctag

SEQ ID NO: 116 - protein, 172 aa, Prunus serotina
MARMSEPLVVGRVIGDVLDCFTPTTKMSVTYNTKLVCNGYELYPSAVTNKPRVEIQGGDMRTFFTLVMTDPDVPGPSDPYLREHLHWIVTDIPGTTDATFGREVVSYEMPRPNIGIHRFVFVLFKQKRRQSVNPPSSRDHFSARSFAAENDLGLPVAAVYFNCQRETAARRR

SEQ ID NO: 117 - DNA, 224 bp, Prunus serotina
  source 1..224: mol_type = genomic DNA
agctacaccttctttctcactcttcctcattctctcctagtcacaaaaaaaatggcaagaatgtctgagcctctggttgttgggagagtgataggagatgttcttgattgtttcaccccaacaacaaaaatgtctgtcacttacaacaccaagctagtctgcaatggatatgagctctatccttctgctgtcaccaacaaacctagagttgagattcaaggagg

SEQ ID NO: 118 - DNA, 144 bp, Prunus serotina
  source 1..144: mol_type = genomic DNA
cacaaaaaaaatggcaagaatgtctgagcctctggttgttgggagagtgataggagatgttcttgattgtttcaccccaacaacaaaaatgtctgtcacttacaacaccaagctagtctgcaatggatatgagctctatccttc

SEQ ID NO: 119 - DNA, 104 bp, Prunus serotina
  source 1..104: mol_type = genomic DNA
tgtctgagcctctggttgttgggagagtgataggagatgttcttgattgtttcaccccaacaacaaaaatgtctgtcacttacaacaccaagctagtctgcaat

SEQ ID NO: 120 - DNA, 63 bp, Prunus serotina
  source 1..63: mol_type = genomic DNA
gggagagtgataggagatgttcttgattgtttcaccccaacaacaaaaatgtctgtcacttac

SEQ ID NO: 121 - DNA, 223 bp, Prunus serotina
  source 1..223: mol_type = genomic DNA
tgagctctatccttctgctgtcaccaacaaacctagagttgagattcaaggaggagatatgagaactttctttactctggtcatgacagacccagatgttcctggccctagtgatccttatttaagggagcacctgcactggattgtgacagatattccaggcaccacagatgccacatttggaagagaggtggtgagctatgagatgccaaggcccaacatt

SEQ ID NO: 122 - DNA, 143 bp, Prunus serotina
  source 1..143: mol_type = genomic DNA
gagattcaaggaggagatatgagaactttctttactctggtcatgacagacccagatgttcctggccctagtgatccttatttaagggagcacctgcactggattgtgacagatattccaggcaccacagatgccacatttgg

SEQ ID NO: 123 - DNA, 103 bp, Prunus serotina
  source 1..103: mol_type = genomic DNA
gagaactttctttactctggtcatgacagacccagatgttcctggccctagtgatccttatttaagggagcacctgcactggattgtgacagatattccaggc

SEQ ID NO: 124 - DNA, 63 bp, Prunus serotina
  source 1..63: mol_type = genomic DNA
tcatgacagacccagatgttcctggccctagtgatccttatttaagggagcacctgcactgga

SEQ ID NO: 125 - DNA, 134 bp, Prunus serotina
  source 1..134: mol_type = genomic DNA
cctggccctagtgatccttatttaagggagcacctgcactggattgtgacagatattccaggcaccacagatgccacatttggaagagaggtggtgagctatgagatgccaaggcccaacattggcatccacag

SEQ ID NO: 126 - DNA, 4519 bp, Prunus persica
  source 1..4519: mol_type = genomic DNA
ctttatcttactgataagatctaaagaaaaccagtaggtggataagatcaaatcactttgccatttactcttcggtaaaatatagcaataattgactggaaatgtgacgcagacagattgcacgtacagattacacagattgcagttccataacaggtgtggttcatgagaatatataataaaaataatatatacaaaaaggtaaacttttaagctcaactcaactgtatcaaccttacataatatttataaaaaaatggaaacgatagaggaaggaagcatcgatcctgagggaaaaaagaggaaattaacaattttataaataaaaaaggaaaaaaaaaaaaaggagcaaagaaagcaaaagaatggaaaattcaaacagggggggtgcatgcagtatagtatagaacactttcacatgaggtccagcttggaatatacagaagaaaaaaaaagtagtttggcttcttgtaatggaaaattcatcttctttctcattgccattcctggaacttctctgaaaatcacttttattttaatctgagttatatatattgagatgacctctcacagcaaaagcagtatagcagcaggccaggcactcatagaggtggagttcctttatgccctcagcctgtcttcaaagatagagaatctctctgaaaatgttgaaattccctctcgtgttccatatttatatatgcacacattcaacattttcttctgattacccaaactgcaaaaggtgaatttcctttataagcttgtgaatgttcagcaagaatcctagaaatttagctgtttttgctgtcttgctttttatatatatatatacatatataatagcatgaattgacagccatggaactgtaaggaccccaccatggcatgcaggattgtatataataatggttttctggcctgaccatggaaaaacatccccccgttttgaataaaaatgttcaataattaagtgcacagccaatcaaatcaagaaccaattccaagggacccaattaatatgagaggcacagatatataacataagtatattatgcatattgtcctaaataagcaaaaattattgcatacaccacatttttctcagttaatcatagaaagagtgttaattagtcgtcataagagcacataattcatgtgttagactcaacatgtcacccaacttattacgtttgattttgatcaggtaaatcattacaagaaaaattggctaatgtgatgaaattaaaagtaaagtttctaaattcgtcacatgtagttagaatatgtgaagaatttatttggtcgtcacttataaaatgatcaagagaccaacagtttttgggaaagaccaaagtcgtcacaaacatgagcgcagttctaggtgggaaagactaacaagtatttgtgatgccaacgggtcacatataaaactctaatgcgtcacaaatattaaatgttgaaatctatagtgaaaaaagagaatacaaaaagaattagaggaaaaatttccctacccaaaagaaaatatatatttcaggaattcttttggtcatcgcatataatgaactatgacaaattatgtgaggatcgggcctcacttattcacatctaatattgaagaaaccatgttatatatagaattaatacaactgttatatatacaggaatttttctggaaacgataccactaaactgtgttgatgtgacatagaaaaaagagggtgggaggtgggggactggctaacaatgttttctagagatgtccgtattctatgcctttatactctttcttcttcttaatgcccgtctcaaaacgcataatagagatctaaagtaactcgcttgaaaacactataaatatacaaccttacaggaattccactaccaagcaatataagaagtacatctctaattcctcctgagttcttatccattaatctttgatcaatagctacactttctttctcactcctcctcattctctctgtctccaaaaaatggcaagaatatctgagcctctggttgttgggagagtgataggagatgttcttgattgtttcaccccaacaacaaaaatgtctgtcacttacaacaccaggctagtctgcaatggatatgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccgggcaccacagatgccacatttggaagagaggtggtgagctatgagatgccaaggcccaacattggcatccacaggtttgtgtttgttctcttcaagcagacaagaaggcagtctgtgaaccctccttcctcaagggatcatttcagtgctcgaagctttgcagctgaaaacgacctgggtcctcctgtcgctgccgtttacttcaattgccagagagaaacggcagctagaagacgctagctagctacccaaaaccccaaagctcctccataatatgttaattttaaataaaattattatcaagtgtgtttcatcatcctcccctgtcgttagagtagtattaggctaaaactactcacatgtaaccagaataatttccagtcaagatagagctaggtgcgtttggctctgtcattttgctgctctctaatcggacttaatgaattaatttgcagaaagtttgaagtctcacctgactatctgtgaaatatctctctatggtgtttttaatttatattttattataaggggacaggagcaaccaaactttgtcctttttaagttagggttttggcttccatttcgactggaaaagtagaagaccctctattattgacttcatctcttcttcccagcttagggttttgggttgcacgtttcaagcacaaaaatatgtgcgatttgacagcagtcagtcacaccacatccagctatgtcgacgcttgtaatgaagaggcaactagttttatttttgcttttgagaacgaagaagcaagtattatagatgcatggtaaagaaaaatacttgattccgtcaaagaaagtagaaattcatactattttattgctaattatattttgatatgtgtgtagagatttctctttttgtatttttgataaattatttttgcatatatgtcgaattgtgatttgcagaaagtaggaatatctcattccatggtaaatttataatttataatatgaattatttagaatatgaaaaataatttactgcaaaagtttcacttggaaacccaattttcatgcaccctagtttttttttttttttttacacaaaccgaacattaaaaaactaaaccctaaaaactttgtcgattaagaaccaaaactttgtcatttgataccaaaaaatccacacattttatgccctaatactatatgagttattaaattattggcctttttatccatgaatttatgggttttatttatttcccctcttcattttgcatatgggtttaaacttttcccaataatggagccttgactgttttgagttgtcaacaaaaagcgataatcaacgggatcttgtgcagtggaatgacattaaattgcaggcaaggggtcacaaattcaaaccccatggcacttagttgtgtgtgtgagaaactctatttcttgtagtttagactattacttgtacttaaagaaaaataaaaattaaacaaagatatacgttatctcatctgaatcgatgcacaagttgcacatgactaaaacaacaatatatatgtccaagtgtttatctataggggcggagctagaattgtaaagttaaggggtcgaaaaagaaaatacaagtatataaaacacttaagtattcaataattatatgtttagagctaaatatttctaattgaatcatgtctaatttgttttagaggaaattttaatttaaattataagttgtttattacaaattctatatattatatataatataatagaagatggataaaaatctaaggaggccagggccactccagaactagaagtggctctgccactgcttatctaattaattaaggatccatatattcaagtccctaaatgcatacacgtattagatttaattacaaccgtatgaaattactctaccaaatcatatggttagatttccctcttgcagttgagagtcggattagccatcaatggacaatctaacataatgatgagacggaattatacaaaaaatatatatgaatgtctagcggatctttaatttgcagtgataatttcaggtattatacttatgtttctccattgcagctgtctacgttcttgagattcaagatgtgtattatacttgacgtattggtagacagtagacataattgtatttcatgaggtacgcatgcaatattatgatgattaacataattatatatattattaatcaatttgaattactttttaagatttcatattggcaggtagct

SEQ ID NO: 127 - DNA, 519 bp, Prunus persica
  source 1..519: mol_type = genomic DNA
atggcaagaatatctgagcctctggttgttgggagagtgataggagatgttcttgattgtttcaccccaacaacaaaaatgtctgtcacttacaacaccaggctagtctgcaatggatatgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccgggcaccacagatgccacatttggaagagaggtggtgagctatgagatgccaaggcccaacattggcatccacaggtttgtgtttgttctcttcaagcagacaagaaggcagtctgtgaaccctccttcctcaagggatcatttcagtgctcgaagctttgcagctgaaaacgacctgggtcctcctgtcgctgccgtttacttcaattgccagagagaaacggcagctagaagacgctag

SEQ ID NO: 128 - protein, 172 aa, Prunus persica
MARISEPLVVGRVIGDVLDCFTPTTKMSVTYNTRLVCNGYELYPSAVTTKPRVEIQGGDMRTFFTLIMTDPDVPGPSDPYLREHLHWIVTDIPGTTDATFGREVVSYEMPRPNIGIHRFVFVLFKQTRRQSVNPPSSRDHFSARSFAAENDLGPPVAAVYFNCQRETAARRR

SEQ ID NO: 129 - DNA, 223 bp, Prunus persica
  source 1..223: mol_type = genomic DNA
atagctacactttctttctcactcctcctcattctctctgtctccaaaaaatggcaagaatatctgagcctctggttgttgggagagtgataggagatgttcttgattgtttcaccccaacaacaaaaatgtctgtcacttacaacaccaggctagtctgcaatggatatgagctctatccttctgctgtcaccaccaaacctagagttgagattcaaggagg

SEQ ID NO: 130 - DNA, 143 bp, Prunus persica
  source 1..143: mol_type = genomic DNA
tctccaaaaaatggcaagaatatctgagcctctggttgttgggagagtgataggagatgttcttgattgtttcaccccaacaacaaaaatgtctgtcacttacaacaccaggctagtctgcaatggatatgagctctatcctt

SEQ ID NO: 131 - DNA, 103 bp, Prunus persica
  source 1..103: mol_type = genomic DNA
tatctgagcctctggttgttgggagagtgataggagatgttcttgattgtttcaccccaacaacaaaaatgtctgtcacttacaacaccaggctagtctgcaa

SEQ ID NO: 132 - DNA, 223 bp, Prunus persica
  source 1..223: mol_type = genomic DNA
ggttgttgggagagtgataggagatgttcttgattgtttcaccccaacaacaaaaatgtctgtcacttacaacaccaggctagtctgcaatggatatgagctctatccttctgctgtcaccaccaaacctagagttgagattcaaggaggagatatgagaactttctttactctgatcatgacagacccagatgttcctggccctagtgatccttatttaagg

SEQ ID NO: 133 - DNA, 143 bp, Prunus persica
  source 1..143: mol_type = genomic DNA
accccaacaacaaaaatgtctgtcacttacaacaccaggctagtctgcaatggatatgagctctatccttctgctgtcaccaccaaacctagagttgagattcaaggaggagatatgagaactttctttactctgatcatgac

SEQ ID NO: 134 - DNA, 104 bp, Prunus persica
  source 1..104: mol_type = genomic DNA
ctgtcacttacaacaccaggctagtctgcaatggatatgagctctatccttctgctgtcaccaccaaacctagagttgagattcaaggaggagatatgagaact

SEQ ID NO: 135 - DNA, 63 bp, Prunus persica
  source 1..63: mol_type = genomic DNA
tagtctgcaatggatatgagctctatccttctgctgtcaccaccaaacctagagttgagattc

SEQ ID NO: 136 - DNA, 223 bp, Prunus persica
  source 1..223: mol_type = genomic DNA
tgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccgggcaccacagatgccacatttggaagagaggtggtgagctatgagatgccaaggcccaacatt

SEQ ID NO: 137 - DNA, 143 bp, Prunus persica
  source 1..143: mol_type = genomic DNA
gagattcaaggaggagatatgagaactttctttactctgatcatgacagacccagatgttcctggccctagtgatccttatttaagggagcacctgcactggattgtgacagatattccgggcaccacagatgccacatttgg

SEQ ID NO: 138 - DNA, 103 bp, Prunus persica
  source 1..103: mol_type = genomic DNA
gagaactttctttactctgatcatgacagacccagatgttcctggccctagtgatccttatttaagggagcacctgcactggattgtgacagatattccgggc

SEQ ID NO: 139 - DNA, 132 bp, Prunus persica
  source 1..132: mol_type = genomic DNA
cctggccctagtgatccttatttaagggagcacctgcactggattgtgacagatattccgggcaccacagatgccacatttggaagagaggtggtgagctatgagatgccaaggcccaacattggcatccac

SEQ ID NO: 140 - DNA, 4519 bp, Prunus avium
  source 1..4519: mol_type = genomic DNA
tggtgcatgcagtatggcatagaacactttcacatgaggtccagcttggaatatacagaaaaaaagtagtttggcttcttgtaatggaaaattcaccttctttctcattgccattcctggaacttctctgaaaatcacttttattttaatctgagttatatatatattgagatgacctctcacagcaaaagctgttgcagcaggccaggcactcattgaggtggagttcctttatgccctcagcctgtcttcaaagatagagaatctttctgaaaatgttgaaattctctcgtgtaccatatttatatatgcacacattcaacattttcttctgcttaccctaactgcaaaaggtgaatttcctttataagcttgtgaatgttcagcaagaatcctagacattttgctatttttgctgtgtattgctttttatatatatatatatacatatataatagcatgaattgacagccatggaaatgtaaggaccccaccatgcaggattgtatataataatggttttctggcctgaccatggaaaaatatccccccgttttgaataaaaatgttcaataattaagtgcacagccaatcaaatcaagaaccaattccaagggacccaattaatatgagaggcacagatatataacatgagtatattatgcatattatcctaaataagcaaaaattattgcgtgcaccacatttttcttagttaatcatagagagattgttaattagtagtataagggctcgttcggcactgcttatctaggaagcacttctacttataattgtttctaccaaaagcgcttttattataaatacgttgaaattttcataagaaattaaagtgcttcttggaaaagcacttgggagtgcttcctgaagatgcacatggtatgtgcttctccagaaagcgcttctatgacccaaaatcgcttctaaacttttctggcaaacattgttagaagcgcttttggttgtcagaaagcgcttttaactgttccaaaagcactcccaaacgagtcctaaatctcaagtcgtgataagatcctaaatttttttcacttgttgctaattaattttgagttggatgttctaactatactatgatattagagcacacaattcatgtgttagactcaacacgtcacccaacttattacgtttgattttgatcagttagatcattacaagaaaatttttgactaatgtcatgaaattaaaattgaagttggtaaattcgtcacatgtgattggaatatgtgaggaatttattttgtcgtcacttataacatggccaagaaaacagcagtttgtgggaaagaccaaagtcctcacaaacatgagcgtgattctaggtgggaaagactaacaattatttgtggtgccaatggggtcacatataaaattttaatgcgtcacaaatattaaatgtgggaatctctagtgaaaaaagaaaatacaaaaagaatgagaggaaaaatttccctacccaaaagaaaatatatatatttctggaattcttttggtcatcacatataattaattatgacaaattatgtgaggatcgggcctcacttattcacatctaatatttaagaaaccatgttatagaaagaattaacatataaaatacattaatacaaccgttatatatataggaatttttctggaaacgataccactaaactctattgatgtgacatagaaaacagagggtgggaggtgggggactggctaacaatgttttctagagatgtccatattctatgcctttatactctttcttcttcttaatgcctgtctcaaaacacataatagagatctaaagtaactcgcttgaaaacactataaatacaaccttacatgaattccatttccaagcaatataagaagtacatctctaattcctcctgagttcttatccattaatctttggtcaatagctacaccttctttttcactcctcctcattctctctttctctcagaaaaatggcaagaacgtctgagcctctggttgttgggagagtaataggagatgttcttgattgtttcaccccaacaacaaaaatgtctgtcacttacaacaccaagctagtctgcaatggatatgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccaggcaccacagatgccacatttggaagagaggtggtgagctatgagatgccaaggcccaacattggcatccacaggtttgtgtttgttctcttcaagcagaaaagaaggcagtctgtgaaccctccttcctcaagggatcatttcagtgctcgaagcttcgcagctgaaaacgacctgggtcttcctgtcgctgccgtctacttcaattgccagagagaaacggcagctagaagacgctagctagctacccagaaccccaaagctcctccataatatgtgaattttaaataaaattattatcaagtgtgtttcatcatcctcccctgtcgttagagtagtattaggctaaaactactcacatgtaaccagaataatttccaatcaagagagagcaatgtgcgtttggctctgtcattttgctgctctccaatcggactttatgaattaatttgcagaaagtttgaagtctcacctgactatctgtgaaatatctctcgatggggtttttaatttatattttattataaggggacaggaggaaccaaacgtccctctttttgtcctttttaagttagggttttggcttccatttcgactggaaaagtagaagagcctctattattgacttcatagttcatctcttcttcccagtttagggttttgggttgcacgtttcaagctgaaaaatatgttcgatttgacagcagtcagtcacaccacatccagctatgtcgactcttgtaatgaagaggcaactagttttttttttgcttttgagaacgaagaagcaagtattatagatgcatggtaaagaaaaatacttgactccgtcaaagaaagtacgaattcctactacttgattgcattagagatttctcttttcgtgtttttgataaattatttttgtatatatgtagaactgtgatttacagaaagtaggaatgcctccttccatggtaaatttatgattaacaatatgatttagaataggaaaaacaatttattgtaaaagtttcacttagaaacccaattttcatgcaccctatttttttttacacaaaccgaacattaagaaactaaaccctaaaaactttgtcaattaagaaccaaaactttgtcatttgataccaaaaaagccacacattttatgcttaagaaccctaatactatatgagttattaaattattggccttttcatccatgaatttatgggttttattcttcattttgcatatgggtttaaacttttcgcaataatggagtctcgactattttgagttgtaaacaaaaagcaatagtcaacggaatctagcgcagtggaaaatgatattaaattgcagacaagaggtcacaaattcgaacccccatggcacttagttgtgtgtgtgagaaaccccctctcttgtagtttagactatcacttgtactgaaagaaaaaaaaaataaacaaagatatacgttatctcatctaaatcgatgcacaagttgcacatgattaaaacaacaatatatatgtccaagtgcttattcacaggggcgaagctagaattgtaaagttgaggggtcaaagaagaaaatacaagtatataaaacacctaagtattcaataattacatgtttagagctaaatatttctaattgagtaatgtctaatttgttttagaggaaattttaacttaaattataagttgtttattacaaattctatatgttatatataatatatctggggggccagagctactccaggactagaagtggctccgccactacttatctaattaattaaggatccatatattcaagtctccaaatgcaaacacatactagatttaattgcaaccttatgaattactctaccaaatcatagggttagatttccctcttgcagttgagcatcggattagccatcaatggacaatctaacataatgatgagacggaattatacaaaatatatatatgaatatctagcggatctttaatttgcagtgataatttcaggtattatacttgacgtattggtagacagtattggtagacagtagacataattgtattatacttgacgtattggggtagacagtattggtagacagtagacttatgttttctccattgcagctgtctaagttcttgaaattcaagatgtgtattatacttgacgtattggtagacagtagacataattgtatttcatgaggtacgcatgcaatattatgatgattaacataattata

SEQ ID NO: 141 - DNA, 519 bp, Prunus avium
  source 1..519: mol_type = other DNA
atggcaagaacgtctgagcctctggttgttgggagagtaataggagatgttcttgattgtttcaccccaacaacaaaaatgtctgtcacttacaacaccaagctagtctgcaatggatatgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccaggcaccacagatgccacatttggaagagaggtggtgagctatgagatgccaaggcccaacattggcatccacaggtttgtgtttgttctcttcaagcagaaaagaaggcagtctgtgaaccctccttcctcaagggatcatttcagtgctcgaagcttcgcagctgaaaacgacctgggtcttcctgtcgctgccgtctacttcaattgccagagagaaacggcagctagaagacgctag

SEQ ID NO: 142 - protein, 172 aa, Prunus avium
MARTSEPLVVGRVIGDVLDCFTPTTKMSVTYNTKLVCNGYELYPSAVTTKPRVEIQGGDMRTFFTLIMTDPDVPGPSDPYLREHLHWIVTDIPGTTDATFGREVVSYEMPRPNIGIHRFVFVLFKQKRRQSVNPPSSRDHFSARSFAAENDLGLPVAAVYFNCQRETAARRR

SEQ ID NO: 143 - DNA, 223 bp, Prunus avium
  source 1..223: mol_type = genomic DNA
agctacaccttctttttcactcctcctcattctctctttctctcagaaaaatggcaagaacgtctgagcctctggttgttgggagagtaataggagatgttcttgattgtttcaccccaacaacaaaaatgtctgtcacttacaacaccaagctagtctgcaatggatatgagctctatccttctgctgtcaccaccaaacctagagttgagattcaaggagg

SEQ ID NO: 144 - DNA, 143 bp, Prunus avium
  source 1..143: mol_type = genomic DNA
tctcagaaaaatggcaagaacgtctgagcctctggttgttgggagagtaataggagatgttcttgattgtttcaccccaacaacaaaaatgtctgtcacttacaacaccaagctagtctgcaatggatatgagctctatcctt

SEQ ID NO: 145 - DNA, 103 bp, Prunus avium
  source 1..103: mol_type = genomic DNA
cgtctgagcctctggttgttgggagagtaataggagatgttcttgattgtttcaccccaacaacaaaaatgtctgtcacttacaacaccaagctagtctgcaa

SEQ ID NO: 146 - DNA, 63 bp, Prunus avium
  source 1..63: mol_type = genomic DNA
gggagagtaataggagatgttcttgattgtttcaccccaacaacaaaaatgtctgtcacttac

SEQ ID NO: 147 - DNA, 223 bp, Prunus avium
  source 1..223: mol_type = genomic DNA
ggttgttgggagagtaataggagatgttcttgattgtttcaccccaacaacaaaaatgtctgtcacttacaacaccaagctagtctgcaatggatatgagctctatccttctgctgtcaccaccaaacctagagttgagattcaaggaggagatatgagaactttctttactctgatcatgacagacccagatgttcctggccctagtgatccttatttaagg

SEQ ID NO: 148 - DNA, 143 bp, Prunus avium
  source 1..143: mol_type = genomic DNA
accccaacaacaaaaatgtctgtcacttacaacaccaagctagtctgcaatggatatgagctctatccttctgctgtcaccaccaaacctagagttgagattcaaggaggagatatgagaactttctttactctgatcatgac

SEQ ID NO: 149 - DNA, 103 bp, Prunus avium
  source 1..103: mol_type = genomic DNA
tgtcacttacaacaccaagctagtctgcaatggatatgagctctatccttctgctgtcaccaccaaacctagagttgagattcaaggaggagatatgagaact

SEQ ID NO: 150 - DNA, 223 bp, Prunus avium
  source 1..223: mol_type = genomic DNA
tgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccaggcaccacagatgccacatttggaagagaggtggtgagctatgagatgccaaggcccaacatt

SEQ ID NO: 151 - DNA, 143 bp, Prunus avium
  source 1..143: mol_type = genomic DNA
gagattcaaggaggagatatgagaactttctttactctgatcatgacagacccagatgttcctggccctagtgatccttatttaagggagcacctgcactggattgtgacagatattccaggcaccacagatgccacatttgg

SEQ ID NO: 152 - DNA, 103 bp, Prunus avium
  source 1..103: mol_type = genomic DNA
gagaactttctttactctgatcatgacagacccagatgttcctggccctagtgatccttatttaagggagcacctgcactggattgtgacagatattccaggc

SEQ ID NO: 153 - DNA, 519 bp, Prunus armeniaca
  source 1..519: mol_type = other DNA
atggcaagaatgtctgagcctttggttgttgggagagtgataggagatgttcttgattgtttcaccccaacaacaaaaatgtctgtcacttacaacaccaggctagtctgcaatggatatgagctgtatccttctgctgtcaccaccaaacctagagttgagattcaaggaggagatatgagaactttctttactctgatcatgacagacccagatgttcctggccctagtgatccttatttaagggagcacctgcactggattgtgacagatattccaggcaccacagatgccacatttggaagagaggtggtgagctatgagatgccaaggcccaacattggcatccacacgtttgtgtgtgttctcttcaagcagaaaagaaggcagtctgtgaaccctccttcctcaagggatcatttcagtgctcgaagcttcgcagctgaaaacgacctggatcttcctgtcgctgccgtttacttcaattgccagagagaaacggcagctagaagacgctag

SEQ ID NO: 154 - protein, 172 aa, Prunus armeniaca
MARMSEPLVVGRVIGDVLDCFTPTTKMSVTYNTRLVCNGYELYPSAVTTKPRVEIQGGDMRTFFTLIMTDPDVPGPSDPYLREHLHWIVTDIPGTTDATFGREVVSYEMPRPNIGIHTFVCVLFKQKRRQSVNPPSSRDHFSARSFAAENDLDLPVAAVYFNCQRETAARRR

SEQ ID NO: 155 - DNA, 173 bp, Prunus armeniaca
  source 1..173: mol_type = other DNA
atggcaagaatgtctgagcctttggttgttgggagagtgataggagatgttcttgattgtttcaccccaacaacaaaaatgtctgtcacttacaacaccaggctagtctgcaatggatatgagctgtatccttctgctgtcaccaccaaacctagagttgagattcaaggagg

SEQ ID NO: 156 - DNA, 133 bp, Prunus armeniaca
  source 1..133: mol_type = other DNA
atggcaagaatgtctgagcctttggttgttgggagagtgataggagatgttcttgattgtttcaccccaacaacaaaaatgtctgtcacttacaacaccaggctagtctgcaatggatatgagctgtatcctt

SEQ ID NO: 157 - DNA, 103 bp, Prunus armeniaca
  source 1..103: mol_type = other DNA
tgtctgagcctttggttgttgggagagtgataggagatgttcttgattgtttcaccccaacaacaaaaatgtctgtcacttacaacaccaggctagtctgcaa

SEQ ID NO: 158 - DNA, 223 bp, Prunus armeniaca
  source 1..223: mol_type = other DNA
tgagctgtatccttctgctgtcaccaccaaacctagagttgagattcaaggaggagatatgagaactttctttactctgatcatgacagacccagatgttcctggccctagtgatccttatttaagggagcacctgcactggattgtgacagatattccaggcaccacagatgccacatttggaagagaggtggtgagctatgagatgccaaggcccaacatt

SEQ ID NO: 159 - DNA, 143 bp, Prunus armeniaca; identical to SEQ ID NO: 151
  source 1..143: mol_type = other DNA

SEQ ID NO: 160 - DNA, 103 bp, Prunus armeniaca; identical to SEQ ID NO: 152
  source 1..103: mol_type = other DNA

SEQ ID NO: 161 - DNA, 132 bp, Prunus armeniaca
  source 1..132: mol_type = other DNA
cctggccctagtgatccttatttaagggagcacctgcactggattgtgacagatattccaggcaccacagatgccacatttggaagagaggtggtgagctatgagatgccaaggcccaacattggcatccac

SEQ ID NO: 162 - DNA, 667 bp, Malus domestica
  source 1..667: mol_type = genomic DNA
ggagtgctattagctcctcctgaattgacttatccattaatcttctctttttacactctctcttctctctctctctctctctctctctctctctctctctctctctctctctctctcttaaaatgaaaagagcctcggagcctctggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaatcaatcaacacaccttcctcgagggatcacttcagcactcgaagcttcgcggctgaaaatgacctgggtcttcctgtcgctgccgtctacttcaacgcgcagagagaaactgcagctagaagacgctagctagtagctgtacccagaactcctcc

SEQ ID NO: 163 - DNA, 519 bp, Malus domestica
  source 1..519: mol_type = other DNA
atgaaaagagcctcggagcctctggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaatcaatcaacacaccttcctcgagggatcacttcagcactcgaagcttcgcggctgaaaatgacctgggtcttcctgtcgctgccgtctacttcaacgcgcagagagaaactgcagctagaagacgctag

SEQ ID NO: 164 - protein, 172 aa, Malus domestica
MKRASEPLVVGRVIGDVLDSFTATTKMSVTYNTKLVCNGLELFPSVVTAKPRVEIQGGDMRSFFTLVMTDPDFPGPSDPYLREHLHWIVTDIPGTTDATFGREVVSYEMPKPNIGIHRFVFVLFKQNQRQSINTPSSRDHFSTRSFAAENDLGLPVAAVYFNAQRETAARRR

SEQ ID NO: 165 - DNA, 491 bp, Malus domestica
  source 1..491: mol_type = genomic DNA
ctctctctctctctcttaaaatgaaaagagcctcggagcctctggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaatcaatcaacacaccttcctcgagggatcacttcagcactcgaagcttcgcggctgaaaatgacctgggtcttcctgtcgct

SEQ ID NO: 166 - DNA, 421 bp, Malus domestica
  source 1..421: mol_type = genomic DNA
cctcggagcctctggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaatcaatcaacacaccttcctcgagggatcacttcagcactcg

SEQ ID NO: 167 - DNA, 354 bp, Malus domestica
  source 1..354: mol_type = genomic DNA
aatgtctgtcacttacaacaccaagctagtctgcaatggacttgagctctttccttctgttgtcacagccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaatcaatcaacacaccttcctcgagggatcacttcagcactcg

SEQ ID NO: 168 - DNA, 246 bp, Malus domestica
  source 1..246: mol_type = genomic DNA
ccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaat

SEQ ID NO: 169 - DNA, 132 bp, Malus domestica
  source 1..132: mol_type = genomic DNA
ccgacccagattttcctggccctagtgatccttatctaagggagcacctgcactggattgtgacagacattccaggcaccacagatgccacatttggaagagaggtggtgagttatgagatgccgaagccca

SEQ ID NO: 170 - DNA, 112 bp, Malus domestica
  source 1..112: mol_type = genomic DNA
ggcaccacagatgccacatttggaagagaggtggtgagttatgagatgccgaagcccaacattggcatccaccggtttgtgtttgttcttttcaagcagaatcaaagacaat

SEQ ID NO: 171 - DNA, 132 bp, Malus domestica
  source 1..132: mol_type = genomic DNA
cctggccctagtgatccttatctaagggagcacctgcactggattgtgacagacattccaggcaccacagatgccacatttggaagagaggtggtgagttatgagatgccgaagcccaacattggcatccac

SEQ ID NO: 172 - DNA, 4342 bp, Fragaria vesca
  source 1..4342: mol_type = genomic DNA
gcaatagagagaaaagaaccgtgaggaggaaaaggaggaatgaaactacttaaaaatataaagagcgaaaagaaataaagggatacgttgaaacattgggcatggtgaatgcagagagttgcagaagagatacagctagcttcggcatatgcagcagagaaaagtactttggcttcttgttatagcgaatccactccttgatatctagtgcttaattgccaattctggaacacttctctgcaacactttattcactttggcatgggttgtagcactcgtctgagatgacctctccatcagcactatcagtgggtgggttcccttttgccttgtgcttctgtctctcgttgctgaaagaagtctttgaattaccaaaataaccaagagaatctctcaaaatggtaaattcccatatgagtatacttgcttgtaaatgagaaaggttctgtggtgcaacacattcagcaaattttcttattcagttttcagtataccctatctggaaaagatgaaggttttctgttactagctgtatgaatcttcaggaagaacagagaatccgagacatttttaatttgctgtttcgttataaaatagcatgaagtgacagctaattcaaggacccggcccacatgcatgatggattgttttttttttttttttttcttttatggcaaagaacttttaagaagaacacactgtattttttttttttgaagaagaataattcagcaggtttacagccaatcaagaactgattccaaagaccatgcatattagagtacgtttggcacattagtatagaattcttatttttataaaataatgtatcatgtattaaatataaaaatttgaatagaatatcttgtattttttttgtttatatgtttctctagccacaaaattcacttgttctagctatcaataaatcacctgtttatccgattcaagaaaaagaaattcacttgtttcttacgattccttaaaatcttaaattgatgcaaggttgaaaagagtgtaataggttattgcactctgtcatttttttttatcaggtaaattacaactgtgtgagtcgaactcataacctctcacttaccaaaaagaggagagactatgttactagactaaatggcacttcatcatctttgtctgttaatttatttgttgaatatccaacatcttagttttcctgcaaggcatttcctatgtaattaaattccaagtatatatgacatcttatcattccttaagcctgatcatttcacaggtctcccaaaacttacctccttcatctttcattgtatgagtgaaaacttcccctcggcaaagccattatcgccaaaacttcttttgagtctcaagtcttaaccattctacattcatgcaagagtacgtcaacatgcatgatcgacggttgaaattgattcacagtttcgggtagtacatcttgtccagaaagaagagaacattttcctcaagaaaaagcattaaaccctttgcattgatgtgacatgcacagagagaaagagaggacgggaggtgcgagagactagctaacaatgttttaaacttctaaatttcttctaatgtcccatgatattctattcatttatactcttcttcttcttcttctctcccaaaacacttgtgagatctagagcaaactagttagcgtactgcagatcactataaatatagaccaatacagagattcacttgcaagcaaaggcaaatattagaagagctaatcctttccccgagtttatccatctttgatttatctgtacaaccttttctcttctccctctttcgagttctaacaatggcaagaatgtcggaacctctagctgttggaagagtcataggagatgttcttgattccttcacccccactacaaaaatgattctcttacaacagcaagctcgtctgcaatggacatgagctcttcccttctgcagtcaccgccaaacctagagttgagattcaaggaggcgacatgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgctgccgtttacttcaatgcacagagagaaacggcagcaagaagacgctagctagctaggcaccttgcagggaggcaggccagatcccaaaagatctcttgctataaccagaattaatcctccctaattaaataaaagagaagtgtgtagtaatgaattaatcctccctttggctaggttattaggctaaaattactcgcatcttaccagaataattttcccaaacggagctgtacgtgagtgtttgtgtgactgtcattagcttttctttttcctctctccggcctggaatgttatgaagtaaatttgttcttagttaatttgatgtaccggtaaagtatctattcaatatagatacatgtctttggttttgaatcatcgttgatcactggaaatctataaaggaacacgtggaacaaacccattatctgtctatttcaattctcttcttctggttaaggtttagggcatccatttcatttatgatgcaattagaatctgtgttcaagctttgtccacccatggagaaaaattcaacctaggttgaatgccacatacaaatctcacccaagtgaaaaaaaacccactttcaacccataaaatgggctatctcattttccaacctaggttggattgttgggagagaagtgacccaggttggatgagaaatccagcccaggttgctttgtgacacctcaaccgagcccacttgcccatttttcacgtggtgtgcatcatcttcttgcctcccacaaacaaacgcgcttggagttggcgttttgcgcacgaaccaacaacagccatgcaatagcgcgtgtattgtgccattttgtcgagacatactgcgagcttcctaacccccccccccccccccccccgacacgtgtcacgtcaccgatgttgctaactttcattttgaatgtaacggtcaaaagatctgagccgttgctggagcaatctgactgttgtaattagctagctttttttttttgaatctgaccgttttggaatattttcaacgatgagaaaaaagtttataacggtaagaaaaaaaatatcccctataacgggaagaaaaaaatattaaaaaaatttgtataatcccctataattaaatacccatcacaattttacacatccgcaccaatttctctaagacgtcaaaatttcctagtctccaaattccgtagctttcatcttctaggttttaccttatttcaatcattctctctcttctcattcgcactcttcacaatgaataatttgtggcaaaggatccgaaaatctcaagaagaagacgactaagatgatctagcaactaacaatatcgttatcgcagccattgctcaccttgaagctgaaaaccaacctcgaggtcgcggatctcgtccaggtcgtcgtccaaaccaacctggagaaagggaggacaaaggcaaaggtatgcttgaagattattttgtcgaacgttctatttttaatgatgaggaattccgagttcgctacagaatgagccacggtgtcttcaaccgcatatgtggtgacctttgcaactacgatcgatattttgttcaaaaaatcgatgcttccaagaaggtcggtctacttccccaacaaaagatgacatcctctttacagatgctcgcctacggcacatccgcagatcaatgtgctgagtattgtcagatgacaaaatctacctctatcgagtgcctccaacgatttacaagaggaattgttgctctttactcagcagattaccttcaagctcttaatccagtcgatctaaaaaaacttcttgctaaaggtgaaaagcgaggttttccagggatgattgtgagcatcgactgcatgcattggcaatggaagaattgtccatccggtaattgggctggtgaatatagtggtagaaaacacatccccactatcattcttaaagcggtcgcattatacgacacttggatatgacacaccttctttggaatacccgggtcatgaaataacctcaatgtcctagcaaaatcctcgttgtttgacgagcttactatcggtca

SEQ ID NO: 173 - DNA, 519 bp, Fragaria vesca
  source 1..519: mol_type = other DNA
atggcaagaatgtcggaacctctagctgttggaagagtcataggagatgttcttgattccttcacccccactacaaaaatgattgtctcttacaacagcaagctcgtctgcaatggacatgagctcttcccttctgcagtcaccgccaaacctagagttgagattcaaggaggcgacatgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgctgccgtttacttcaatgcacagagagaaacggcagcaagaagacgctag

SEQ ID NO: 174 - protein, 172 aa, Fragaria vesca
MARMSEPLAVGRVIGDVLDSFTPTTKMIVSYNSKLVCNGHELFPSAVTAKPRVEIQGGDMRSFFTLVMTDPDVPGPSDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 175 - DNA, 224 bp, Fragaria vesca
  source 1..224: mol_type = genomic DNA
tgagctcttcccttctgcagtcaccgccaaacctagagttgagattcaaggaggcgacatgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacatag

SEQ ID NO: 176 - DNA, 143 bp, Fragaria vesca
  source 1..143: mol_type = genomic DNA
gagattcaaggaggcgacatgagatcattcttcactctggtaatgacagacccagatgttcctggccctagtgatccttatttgaaagagcacctgcactggattgtgacagacattcctggcaccacagatgctacatttgg

SEQ ID NO: 177 - DNA, 103 bp, Fragaria vesca
  source 1..103: mol_type = genomic DNA
gagatcattcttcactctggtaatgacagacccagatgttcctggccctagtgatccttatttgaaagagcacctgcactggattgtgacagacattcctggc

SEQ ID NO: 178 - DNA, 63 bp, Fragaria vesca
  source 1..63: mol_type = genomic DNA
taatgacagacccagatgttcctggccctagtgatccttatttgaaagagcacctgcactgga

SEQ ID NO: 179 - DNA, 222 bp, Fragaria vesca
  source 1..222: mol_type = genomic DNA
tgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacc

SEQ ID NO: 180 - DNA, 140 bp, Fragaria vesca
  source 1..140: mol_type = genomic DNA
cctggccctagtgatccttatttgaaagagcacctgcactggattgtgacagacattcctggcaccacagatgctacatttggaagagaagtggtgagctacgagatgccaaggccaaacataggcatccacaggtttgt

SEQ ID NO: 181 - DNA, 100 bp, Fragaria vesca
  source 1..100: mol_type = genomic DNA
tttgaaagagcacctgcactggattgtgacagacattcctggcaccacagatgctacatttggaagagaagtggtgagctacgagatgccaaggccaaac

SEQ ID NO: 182 - DNA, 60 bp, Fragaria vesca
  source 1..60: mol_type = genomic DNA
ggattgtgacagacattcctggcaccacagatgctacatttggaagagaagtggtgagct

SEQ ID NO: 183 - DNA, 132 bp, Fragaria vesca
  source 1..132: mol_type = genomic DNA
cctggccctagtgatccttatttgaaagagcacctgcactggattgtgacagacattcctggcaccacagatgctacatttggaagagaagtggtgagctacgagatgccaaggccaaacataggcatccac

SEQ ID NO: 184 - DNA, 4534 bp, Arabidopsis thaliana
  source 1..4534: mol_type = genomic DNA
cggggatagagaagattcttttgggggggtttggggtcgtgactcatgtcgtaagttgttgcttgctacatcttttggattaagcttcacctaagctaaatagctagttgatacgttgtctactttttttttttttttgtcgttgatacgttgtctacttgtattgagcacattgatgtttctcttttctttttgaaaattcgatttatctttttttttttttctaaattgttaaagggttgtcttgtttggagaatttgttgtagacaactttatataagtttgatatattttgctttgtgcctttattctgattaattaccaaatcttaataaagcatcaatcaagactttctaatataagaaaataaataaattattatttagtggagaatccataataagttaataatcattatattcaataggacaaaacctttgggtgtgtttatattagtctacggattttctgatttgtataacacttttggtatttcagattaatagagtattatggttgaaatatgcattcgtaaaatcttcggaagttttaaggttttgtaactaataatcatatatagaatcccaatttgaagaatcggttcttcacgttggttttattgattcgataaaaagaagtttttttaagaatcaatgcgacaatgcgttgtcgttccacgagcaaggagtcccaaatctgcgatatgtttctttcaaatagcctctgagctaggcctgggcattcagtttcggttcggttgcgcatggtttcggttatttcggttatagtaatataagaaccatttggatatttgaggaattttggttcggttcggttcggatatctttcggttcggttcggtttggatattaaacttggtaaccataaataaactaaaaccgaaataaccgactaaaaaccgaaacaaccaaaaaatttgaccaataaccgatatagaacgtacgtaaaaaagagaaatattcacaactcacaaagatggtccatactctccataatattaaaacacctgattcacaactcacgaaattcaacttgcaaactaataaagagaaaaacatagttaagtgtcaatacatactgtgatatattatctatcatacatatatgctttggttttcatttggttatcggttattaacctaaccgaaaccgaaaccgaaatctaagacatataatattcaaccggttattttaagctatccaaacctgaaccgaaccatgtttttcggttcgattcggttcggttaatcggttagcggtttttttgcccaggcctactctgagcaataattgtatccggagttgtaatagaatcaaagtacgatgagagtgtttttatgacaaatatcttaatcttggccaattatatgttctactgaaattctttttgaattcatcgaccagtgagacttaaaaatagctttttattcgccgaggtatatatagctaggaattttgtcgaaatttagacgttagtgggttttgttcttcgtgacacaaaagatattctatatattaacgaaatctagcgatcgatatggtatttatataaagtcttggtcatagataggggttgaaacttgaaaccatgcatgatatgccaatgttgctgaagcagtcaatgttgctgaagaagtcaaacgtaattatatagtgaataccaaaaaagtgatatttcttaattcaattaaatataattatagttttaaatcacctaaaataagttacttattaaaaccccccaaatttactttaatatagttggtgtacatgtttgagaaagcaaacaaaaagaaaaagaaaaagaaaaaaaaaagagaaagaggttagtacacataattgggaattaatgtctattgattcttttatctttctctctctctctaagacggaaaacccctataaatagatgtctcggtcgtctctttgtctcccaaatcactacaaatctctcttttcctctaagttaacaaaagaaaatggagaatatgggaactagagtgatagagccattgataatggggagagtggtaggagatgttcttgatttcttcactccaacaactaagatgaatgttagttataacaagaagcaagtctccaatggccatgagctctttccttcttctgtttcctccaagcctagggttgagatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatcccttcgagagatcacttcaacactcgtaaatttgcggtcgagtatgatcttggtctccctgtcgcggccgtcttctttaacgcacaaagagaaaccgctgcacgcaaacgctagtttcatgattgtcataaactgcaaaaatgaaagaagaaaatttgcatgtaatctcatgtttatttgtgttctgaatttccgtactctgaataaaaactgccaaagatgagttgaatccgaaatatcaattgagtttacagaagtattgataacgatctgtcgattatcagaataaaaactagattaattgcatatcatgtttagcattgtaatactacaaaaatagtaaactcttgattaattaataaaatctaagttgctgtagtatataaatcattaaatcctcatacatggcttgataggtcacatcacatgtagtgaaccttattatatgataaacgtggagatacggaaaaggatagttaaacgatgaaaactttttttagttctggtcaaagtgacaagaccttgatgaccatctaaaatatgatcctctcctcttcttactcggcgatcatattcatatctattttcacggctggtattgtgacaaacaaccaccttttattttcaaaaaaaaatctaataggcatatgtatatttctctatagggaccctttctctatatatatatttattacattccaaaattcaacgtcaaaaatatagatttattgggtataaatccctcactagcgtagacatgtccgcggtaaacaaaaaatgtctctgcctacaaactatataattcgaaacatattagatagaaaagtcagtcttcaataaaatatttattatatgtatatatgaggtatgagatatagttttttgtcaagagttttaagaaaaaatatctatattcaaccgaataaccattaaataacaagtcttaatcattaaaatgcttacaatattttcatatttttacttaagattttactttttgctaccttgcaagttgcaaaagtataattaagtaattaacagtctagtttttgacaacaagaagaaattagcattctacattgattcagtgttcaaatggagaaaagttttttttaaatggattggccattttgggtgtataaagaatatagtacagcctgaatgatagacccatctttgagaaatgagagatgcaagtgggaaatattaatttcaacggtaaactcaagagagatcacgtgtcattaattcagtggttagcggctaagtactgttacaattatcctcaacgccgaccaacttctggttctctacatgtatattcgctatcatataaacaaatctagcaaaatacgactaacttagtaacttgtttcgattttgttagattactattacatagtttggattctcgtttattaacaaattaccaaatatcataaacatttttcgttccagctcaatgactaaaggtattgaaatcgagtttaagttcaatgtgggtttcaatcattttattttatttttgcttaagaatagtcaaagcaaaattgatcaaaaacaaactggttttggtttcattgtgaattaaaaaaaaaaagggataagataagggttatctacacatgtgccataacaaaatcttatgtaattattgcttccaagacttcagagctaaaataaataaagaaaaaactgaagaaaaaacaaagaagtatatacagtacacctacgtaggcaaattgattcgctaccaaatcaagggcatgcaaagactttaggatttgcctgaaaaagacactgtgaactagaacattatatagagttttagggtttcagtaacacataatgtgcctgtgttgtgtttagccacttatggttattttcatagctaaatatttttccaaaagtgaaacaccatttttatttataaaatgactggcccctcgcgtttggtgttattacaaatcaacacgtgctctgttccattccatccctgattttactcaaaattttgctacatctaaatatatttcttattgccaaaattgagatggtgattggtgtcagtatttgggtatgaatcgttttgaactaaaagtctaaaacattat

SEQ ID NO: 185 - DNA, 534 bp, Arabidopsis thaliana
  source 1..534: mol_type = other DNA
atggagaatatgggaactagagtgatagagccattgataatggggagagtggtaggagatgttcttgatttcttcactccaacaactaagatgaatgttagttataacaagaagcaagtctccaatggccatgagctctttccttcttctgtttcctccaagcctagggttgagatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatcccttcgagagatcacttcaacactcgtaaatttgcggtcgagtatgatcttggtctccctgtcgcggccgtcttctttaacgcacaaagagaaaccgctgcacgcaaacgctag

SEQ ID NO: 186 - protein, 177 aa, Arabidopsis thaliana
MENMGTRVIEPLIMGRVVGDVLDFFTPTTKMNVSYNKKQVSNGHELFPSSVSSKPRVEIHGGDLRSFFTLVMIDPDVPGPSDPFLKEHLHWIVTNIPGTTDATFGKEVVSYELPRPSIGIHRFVFVLFRQKQRRVIFPNIPSRDHFNTRKFAVEYDLGLPVAAVFFNAQRETAARKR

SEQ ID NO: 187 - DNA, 371 bp, Arabidopsis thaliana
  source 1..371: mol_type = genomic DNA
gaatgttagttataacaagaagcaagtctccaatggccatgagctctttccttcttctgtttcctccaagcctagggttgagatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatcccttcgagagatcacttcaacactcgtaaatttgcggtcgagt

SEQ ID NO: 188 - DNA, 331 bp, Arabidopsis thaliana
  source 1..331: mol_type = genomic DNA
gagctctttccttcttctgtttcctccaagcctagggttgagatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatcccttcgagagatcacttcaacactcgtaaatttgcggtcgagt

SEQ ID NO: 189 - DNA, 252 bp, Arabidopsis thaliana
  source 1..252: mol_type = genomic DNA
agatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatccctt

SEQ ID NO: 190 - DNA, 174 bp, Arabidopsis thaliana
  source 1..174: mol_type = genomic DNA
tgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttca

SEQ ID NO: 191 - DNA, 136 bp, Arabidopsis thaliana
  source 1..136: mol_type = genomic DNA
tccaggtcctagtgacccctttctaaaagaacacctgcactggatcgttacaaacattcccggcacaacagatgctacgtttggcaaagaggtggtgagctatgaattgccaaggccaagcatagggatacatagg

SEQ ID NO: 192 - DNA, 132 bp, Arabidopsis thaliana
  source 1..132: mol_type = genomic DNA
ccaggtcctagtgacccctttctaaaagaacacctgcactggatcgttacaaacattcccggcacaacagatgctacgtttggcaaagaggtggtgagctatgaattgccaaggccaagcatagggatacat

SEQ ID NO: 193 - protein, 44 aa, Rubus occidentalis
PGPSDPYLKEHLHWVVTDIPGTTDATFGREVVSYEMPRPNIGIH

SEQ ID NO: 194 - protein, 44 aa, Pyrus pyrifolia
PGPSDPYLREHLHWIVTDIPGTTDATFGREVVSYEMPKPNIGIH

SEQ ID NO: 195 - protein, 44 aa, Prunus serotina
PGPSDPYLREHLHWIVTDIPGTTDATFGREVVSYEMPRPNIGIH

SEQ ID NO: 196 - protein, 44 aa, Fragaria vesca
PGPSDPYLKEHLHWIVTDIPGTTDATFGREVVSYEMPRPNIGIH

SEQ ID NO: 197 - protein, 44 aa, Arabidopsis thaliana
PGPSDPFLKEHLHWIVTNIPGTTDATFGKEVVSYELPRPSIGIH

SEQ ID NO: 198 - DNA, 23 bp, synthetic construct
  source 1..23: mol_type = other DNA
gtagttggggtgaaggaatcaag

SEQ ID NO: 199 - DNA, 23 bp, synthetic construct
  source 1..23: mol_type = other DNA
gtggtgactgcagaaggtaagag

SEQ ID NO: 200 - DNA, 23 bp, synthetic construct
  source 1..23: mol_type = other DNA
atttgtaggtgatgacagaccca

SEQ ID NO: 201 - DNA, 23 bp, synthetic construct
  source 1..23: mol_type = other DNA
aaggagcacctgcactggtataa

SEQ ID NO: 202 - DNA, 23 bp, synthetic construct
  source 1..23: mol_type = other DNA
taggtgatgacagacccagatgc

SEQ ID NO: 203 - DNA, 23 bp, synthetic construct
  source 1..23: mol_type = other DNA
attgtaagttataccagtgcagg

SEQ ID NO: 204 - DNA, 23 bp, synthetic construct
  source 1..23: mol_type = other DNA
gttattttcacaggtaatgacag

SEQ ID NO: 205 - DNA, 23 bp, synthetic construct
  source 1..23: mol_type = other DNA
acaggtaatgacagacccagatg

SEQ ID NO: 206 - DNA, 23 bp, synthetic construct
  source 1..23: mol_type = other DNA
aaataaggatcactagggccagg

SEQ ID NO: 207 - DNA, 23 bp, synthetic construct
  source 1..23: mol_type = other DNA
ttgttggggtgaaacaatcaaga

SEQ ID NO: 208 - DNA, 23 bp, synthetic construct
  source 1..23: mol_type = other DNA
gtggtgacagcagaaggatagag

SEQ ID NO: 209 - DNA, 23 bp, synthetic construct
  source 1..23: mol_type = other DNA
ttcaccccaactaccaaaatgat

SEQ ID NO: 210 - DNA, 23 bp, synthetic construct
  source 1..23: mol_type = other DNA
ttagagacgtgcctcgtagtgta

SEQ ID NO: 211 - DNA, 20 bp, synthetic construct
  source 1..20: mol_type = other DNA
ggcaagaatgtcggaaccct

SEQ ID NO: 212 - DNA, 20 bp, synthetic construct
  source 1..20: mol_type = other DNA
ggcccaacatcggcatccac

SEQ ID NO: 213 - DNA, 20 bp, synthetic construct
  source 1..20: mol_type = other DNA
ggcaccacagatgctacatt

SEQ ID NO: 214 - DNA, 5843 bp, synthetic construct
  source 1..5843: mol_type = other DNA
ccaccatatccaaatctctaaaccytaaaccytaaaatycaccatacacctaatccctcgaccctarayycaaaatyyacyataccyctaatytttcaaycctaaacgycacaaatcattaaycaaaaaytmtaaactctaaytcctcaaccttaacccttaaaytgtaaattytaaactcataaccttaaattytaaaccctaaatcctaaacgtaaatcccccaaaayyctagctacaagtaattyctcaagtcttcytaattawtgatyatgcyyaaattgggacatattacttgatatcttctcaactargttgttatctactaacatgccttatcatttgcactaaatraatatatgaccayattctttcctaaatycaaattactaagcattatcaatttrctatttagttgtctttrttttytcyatgatgatraggtagtaacattgtcatstraatcraastttagtgaaagtaartgatttgaktcgtttacaatcaattaaacaatagttctttcatgtggttataawcaatwrttccataattgtaatctcaattaaarttcatctaaattrtacctcctttcyaattaagtttacattgctaagtttttttttttataatatatatatatatatatatatattaaagtgcatgattctatttgattgataaaaaatatatttataaagataaaattataattgtaaggtaattcattrataaratrtttatatataaagctattaattataattgtaagtataattaattaacaagttttaaagttgagtaagcaatagccattgaatctgagatgagtatgatagactttgcataaaatgastaggcaattgtttaatgatgtgtatatgatgtggcatgccasatatgattgaracctcyacatcttaggtcttagagcatytycaasagttttcctaaaaaatctttattatagaaattcaaaagttaaaatwtctataatttttattttgcarttccaayagtttttctatcatattctctataatttattatctacaattaaatatggtattatttttaaatttaaaaagtataatcaaataattaatratgaaaatgaatatatttggaaagaggaaagagaakagggtaaaatttttyaaaaagatgtttcyctataattttagagtttgctrtaaatatagagattyttattttctctcttctcactatctctaaaatagagatargtatagggaarctattggagtaaaaaaatactcaatttttcctaaactrgagaaaaaataaattatagggaagctcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaacgtattwttgctacaagaaaactawgtcatttttgaaaacaaaacctgkatttgtgaagatatcaagcacaattttttttttctttttgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctctcyttcgcactcttcttctagtttaagtttagggttttggtktccwtttcatttsaacgggaaaatcagaaggaatcaacrcgatgacccaatctagtctgcgtgcgggccccacactgtcatgtccgatcttaaataactaattaaccgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgacaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttaaaattcgttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctatcggagatggagggtgcggtggtgatccgctaggctcaggagcagcagcggagagcagtgatgggattrcaactgcggctcgagcackggcycgakgacgacrycgrygrcrtgagatgggtgcagagagaagagaagaaaggaaaaggaaaagaagagaaattcggtgcccgaggcgaactgaaattttaaggcctaattttttctatataaatgtatacatttatttttagtataatttttttcattaaatagagaaatgcgtaattattyaattaatcagagtaatttgtggagagtatgaaaaaatttaagacatytttgaaatcgtgaaaattaatacttaagttgaattcttaaatctcttcattaattt

SEQ ID NO: 215 - DNA, 1113 bp, synthetic construct
  source 1..1113: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtgatgacagacccagatkctcctggccctagtgatccttatttgaaggagcacctgcactgggttgtgacagacattccaggcaccacagatgctacatttggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 216 - protein, 172 aa, synthetic construct
MARMSEPLVVGRVIGDVLDSFTPTTKMIVTYTTRHVSNGYELLPSAVTTKPRVEIQGGDMRSFFTLVMTDPDXPGPSDPYLKEHLHWVVTDIPGTTDATFGREVVSYEMPRXNIGIHRFVFVLFRQKRRQSVNPPXSRDXFNTRSFAAENDLGLPVAAVYFNXQRETAARRR

SEQ ID NO: 217 - DNA, 295 bp, synthetic construct
  source 1..295: mol_type = other DNA
tctctctctctctctctckctmtctctctctctctcgtaagtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatcttttttttttgtctgggttcgtyttttwaccaattgtgt

SEQ ID NO: 218 - DNA, 215 bp, synthetic construct
  source 1..215: mol_type = other DNA
gtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacat

SEQ ID NO: 219 - DNA, 175 bp, synthetic construct
  source 1..175: mol_type = other DNA
gtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatca

SEQ ID NO: 220 - DNA, 135 bp, synthetic construct
  source 1..135: mol_type = other DNA
gaagggtcattggagatgttcttgattccttcaccccaactaccaaaatgattgtcacttacactacgaggcacgtctctaatggatatgagctcttaccttctgcagtcaccaccaaacctagagttgagattc

SEQ ID NO: 221 - DNA, 95 bp, synthetic construct
  source 1..95: mol_type = other DNA
cttgattccttcaccccaactaccaaaatgattgtcacttacactacgaggcacgtctctaatggatatgagctcttaccttctgcagtcaccac

SEQ ID NO: 222 - DNA, 223 bp, synthetic construct
  source 1..223: mol_type = other DNA
tctctctctctctctctckctmtctctctctctctcgtaagtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggt

SEQ ID NO: 223 - DNA, 143 bp, synthetic construct
  source 1..143: mol_type = other DNA
gtcgtaawaatggcaagaatgtcggaacccttggttgttggaagggtcattggagatgttcttgattccttcaccccaactaccaaaatgattgtcacttacactacgaggcacgtctctaatggatatgagctcttaccttc

SEQ ID NO: 224 - DNA, 103 bp, synthetic construct
  source 1..103: mol_type = other DNA
gtcggaacccttggttgttggaagggtcattggagatgttcttgattccttcaccccaactaccaaaatgattgtcacttacactacgaggcacgtctctaat

SEQ ID NO: 225 - DNA, 63 bp, synthetic construct
  source 1..63: mol_type = other DNA
gaagggtcattggagatgttcttgattccttcaccccaactaccaaaatgattgtcacttaca

SEQ ID NO: 226 - DNA, 223 bp, synthetic construct
  source 1..223: mol_type = other DNA
ggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatcttttttttttgtctgggttcgtyttttwaccaattgtgt

SEQ ID NO: 227 - DNA, 142 bp, synthetic construct
  source 1..142: mol_type = other DNA
accccaactaccaaaatgattgtcacttacactacgaggcacgtctctaatggatatgagctcttaccttctgcagtcaccaccaaacctagagttgagattcaaggaggtgacatgagatcattcttcactctggtataca

SEQ ID NO: 228 - DNA, 103 bp, synthetic construct
  source 1..103: mol_type = other DNA
tgtcacttacactacgaggcacgtctctaatggatatgagctcttaccttctgcagtcaccaccaaacctagagttgagattcaaggaggtgacatgagatca

SEQ ID NO: 229 - DNA, 63 bp, synthetic construct
  source 1..63: mol_type = other DNA
acgtctctaatggatatgagctcttaccttctgcagtcaccaccaaacctagagttgagattc

SEQ ID NO: 230 - DNA, 220 bp, synthetic construct
  source 1..220: mol_type = other DNA
gagatcattcttcactctggtgatgacagacccagatkctcctggccctagtgatccttatttgaaggagcacctgcactgggttgtgacagacattccaggcaccacagatgctacatttggaagagaggtggtgagctatgagatgccaaggccyaacatcggcatccacaggtttgtgtttgttctcttcaggcagaaacgaaggcagtcagtgaac

SEQ ID NO: 231 - DNA, 140 bp, synthetic construct
  source 1..140: mol_type = other DNA
cctggccctagtgatccttatttgaaggagcacctgcactgggttgtgacagacattccaggcaccacagatgctacatttggaagagaggtggtgagctatgagatgccaaggccyaacatcggcatccacaggtttgt

SEQ ID NO: 232 - DNA, 100 bp, synthetic construct
  source 1..100: mol_type = other DNA
tttgaaggagcacctgcactgggttgtgacagacattccaggcaccacagatgctacatttggaagagaggtggtgagctatgagatgccaaggccyaac

SEQ ID NO: 233 - DNA, 60 bp, synthetic construct
  source 1..60: mol_type = other DNA
gggttgtgacagacattccaggcaccacagatgctacatttggaagagaggtggtgagct

SEQ ID NO: 234 - DNA, 5840 bp, synthetic construct
  source 1..5840: mol_type = other DNA
ycaccatayccaaatctctaaaccytaaaccctaaaattcaccataccyctaatcyctcraccctaratacaaaatccacyatacyyctaatycctcaaycctaaacgycacaaatcattaaycaaaaactctaaactctaaytcctcaaycttaaccyttaaaytgtaaattywaaactcataaccttaaattytaaaccctaaatmctaaacgtaaccyccctaaaayyctarctacaagtaattyctcaagtcttcytaattawtratyaygcyyaaattgggacatattayttgatatcttctcaactrggttgttatctactaacatgccttatcatttgyaytamayraatatatgaccayatyctttcmtaaatycaaawtactaarcattwtcaatttgctatttagttgtctttrttttctccatgatgatragrtagtaacattgtcaygtgaatcraagtttagtgaaatcaaatratttgattcgtttgyaatyaattamacaatagttctttcatgtggttataaacaatagttccataattgtaatctcaattaaagttcatctaaattatayctcctttccaattaagtttakattgctargtttttttttttataatatatatatatatatatatatattamagtgcatgattctatttgattgataaaaaatatatttataaagataaaattataattgtaaggtaattcattgataaratatttatatstaaarctattaattataattgtaagtataattaattrayaagttttaaarttgagtaagcaatagycattgaatctdagatgagtwtratagactnttgcmtaaaatgagtannnnattkyttaatgatgtgtatatgatgtggyatgcyatatatgattgagacctccacatcttaggtcttagagcatctycaabagttttcctaaaaaatctttattatagrgaytcaaaagttaaaatytctatartttttcttycatarttccaacagtttttctatcatattctctataatttattatctacaattaaatatggtattatttttaaatttaaaaagtataatcaaataattaatgatgaaaatgaatatatttggaaagaggaaagagaakagggtaaaawtttccaaaaagatgtttcyctataattttagagtttgctrtaaatatagagatttttrttttctctctyctcactatctctaaaatagagataggtatagrraarctattggagtaaaaaaatacwcaatttttcctaaactagagaaaaaataaattatagggaagctyttggagatgctcttaatgaagtcaacratcyttttccattcagcatacatgymataccttatagcctcatttcacatatatttcacccaaaacttrcctccttaattacctctttgsgtagtactagagggtctcaattaasgcctaactttygaggtctcaattaaaaygtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattgttaaagtgttgtttmaaaaatggtgtaccaaamgtattwttgctacaagaaaacwawgtcatttttgaaaacaaaacctgkatttgtgaagatatcaarcrcaannttttttttttctttttgaaaaccaacacgtactaagagtcatgcaagagtcaacataatcaatggttcgatttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcaacacacttasagatctaaagcaagtactggttagcgaacttgaaattactataaatatagagcaatacagggaaaggcaattataagaagtacaaatgcctcctccttcctgagttwtcagttttcttatccatctttgatttacctttacgactctctctctctctctctctctctctctctctctctctctcgtaagtcgtaataatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatctttttttttttgtctgggttcgtytttttaccaattgtgtkktktcagatctacgaaatcgcttttaagttaattraatttgatttgtaggtgatgacagacccagatkctcctggccctagtgatccttatttgaaggagcacctgcactggtataacttacaatcaaacctagaaactaaaaaaacaaagacaataaacattcaattaattaaattcagaattaattataaatgttgtcacattcagtkcgttcaattcggatttagagtctaaaaacawaagcaatgcttgtytccraatttgaagaaaayattagtttttttttttrttacattggagggawtgaacctttaacctagtctaattctaattcaattactcaccaacccataytayttrtgcyaactctaagggmttggaaaacattagtttcttgaatatgtgtgtgtatatatataaatatatatatatagatattaatgtgaatttsttttctyttcattacatggtcatatttatgtttttggaattttagaatataattgratctagaaattaaagcaagaaaaaagtgcttatgaaaatctgaactagaaaagctytacaattatggagtatatacatatcmagctgggagctaacagttttggtttaaattgaactaaacagggttgtgacagacattccaggcaccacagatgctacatttggttggttaattcactgctagttcagctaccagttatttagtgatcttatagctagagaagatatatcagacaatatcagttatcratcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcacttcaacacccgaarcttcgcagccgaaaatgacctcggtcttcctgtcgctgccgtttacttcaatgcrcagagagaaacagcagcgagaagacgctagctagctagccgcaggaggcaggccagacycccaaatttccaggctataaccatartatctaattaaataaaagaaaagggtggttaatgatttaatcctccctattgttaggttcctaggctaaaagtacttaakttcccaragagagcygtgagtgtttgtgcgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctstcyttcgcactcttcttstagtttaagtttagggttttggtktccatttcatttsaacargaaaatcakaaggaatcaacrcgatgacccaatctagtctgcgtgcgggcyccacactgtcatgtccgatcttaaataactaattaaccgttggtattgtatattcaaaagtaagtggaaaaatattaattatgttagatgctatatcaataaactttagtagsacraaaaaataaattaaaaatgaaacacaacatcattttgacatgacacaagcatgatakttacattacaattaaagcaacaaattcatattatgtcaatgttttttttacatttctcgttcacaaagtatttttttttttgagaaaataacataaaytttattaattaatataaaaggattacaatcctaagcatgaacatcytaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgayaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttawaattcgttaacatttagaaagaaataccactaaattgatgcttagttartatacaatttctaattccacttggttagatwatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggaagggaagagagmgctgggttcgtgaggtkgctrtcggagatggagstgcrgcggtgatmmgctaggctcaggaggagtagcggagagcagtgatgggattrcarstgmggctcgagcackggctcgaggacgacrccgrcggcgtgagatgggtgcagagagaagagaagaaaggaaaaggaaaagaagagaaattcggtggccgaggcgaactgaaattttaagryctaattttttctatrtaaatgtatacatttatttttggtataannnnnnnnttywttwmattraatagaraaatgcrtaattattyaattaatyrgagtaatttgtggagartatgaaaaaatttaagayatmtttraaatygtraaaattaatacttaagttgaattcttaaatctcttcattaattt

SEQ ID NO: 235 - DNA, 522 bp, synthetic construct
  source 1..522: mol_type = other DNA
atggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtgatgacagacccagatkctcctggccctagtgatccttatttgaaggagcacctgcactggtgggttgtgacagacattccaggcaccacagatgctacatttggaagagaggtggtgagctatgagatgccaaggccyaacatcggcatccacaggtttgtgtttgttctcttcaggcagaaacgaaggcagtcagtgaaccctccttcmtcaagggatcacttcaacacccgaarcttcgcagccgaaaatgacctcggtcttcctgtcgctgccgtttacttcaatgcrcagagagaaacagcagcgagaagacgctag

SEQ ID NO: 236 - protein, 173 aa, synthetic construct
MARMSEPLVVGRVIGDVLDSFTPTTKMIVTYTTRHVSNGYELLPSAVTTKPRVEIQGGDMRSFFTLVMTDPDXPGPSDPYLKEHLHWWVVTDIPGTTDATFGREVVSYEMPRXNIGIHRFVFVLFRQKRRQSVNPPXSRDHFNTRXFAAENDLGLPVAAVYFNXQRETAARRR

SEQ ID NO: 237 - DNA, 295 bp, synthetic construct
  source 1..295: mol_type = other DNA
tctctctctctctctctctctctctctctctctctcgtaagtcgtaataatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatctttttttttttgtctgggttcgtytttttaccaattgtg

SEQ ID NO: 238 - DNA, 215 bp, synthetic construct
  source 1..215: mol_type = other DNA
gtcgtaataatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacat

SEQ ID NO: 239 - DNA, 223 bp, synthetic construct
  source 1..223: mol_type = other DNA
tctctctctctctctctctctctctctctctctctcgtaagtcgtaataatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggt

SEQ ID NO: 240 - DNA, 143 bp, synthetic construct
  source 1..143: mol_type = other DNA
gtcgtaataatggcaagaatgtcggaacccttggttgttggaagggtcattggagatgttcttgattccttcaccccaactaccaaaatgattgtcacttacactacgaggcacgtctctaatggatatgagctcttaccttc

SEQ ID NO: 241 - DNA, 223 bp, synthetic construct
  source 1..223: mol_type = other DNA
ggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatctttttttttttgtctgggttcgtytttttaccaattgtg

SEQ ID NO: 242 - DNA, 143 bp, synthetic construct
  source 1..143: mol_type = other DNA
accccaactaccaaaatgattgtcacttacactacgaggcacgtctctaatggatatgagctcttaccttctgcagtcaccaccaaacctagagttgagattcaaggaggtgacatgagatcattcttcactctggtatacat

SEQ ID NO: 243 - DNA, 221 bp, synthetic construct
  source 1..221: mol_type = other DNA
aactagaaaagctytacaattatggagtatatacatatcmagctgggagctaacagttttggtttaaattgaactaaacagggttgtgacagacattccaggcaccacagatgctacatttggttggttaattcactgctagttcagctaccagttatttagtgatcttatagctagagaagatatatcagacaatatcagttatcratcaatwtwtattc

SEQ ID NO: 244 - DNA, 140 bp, synthetic construct
  source 1..140: mol_type = other DNA
agctgggagctaacagttttggtttaaattgaactaaacagggttgtgacagacattccaggcaccacagatgctacatttggttggttaattcactgctagttcagctaccagttatttagtgatcttatagctagaga

SEQ ID NO: 245 - DNA, 100 bp, synthetic construct
  source 1..100: mol_type = other DNA
ggtttaaattgaactaaacagggttgtgacagacattccaggcaccacagatgctacatttggttggttaattcactgctagttcagctaccagttattt

SEQ ID NO: 246 - DNA, 60 bp, synthetic construct
  source 1..60: mol_type = other DNA
gggttgtgacagacattccaggcaccacagatgctacatttggttggttaattcactgct

SEQ ID NO: 247 - protein, 42 aa, synthetic construct
PSDPYLKEHLHWVVTDIPGTTDATFGREVVSYEMPRXNIGIH

SEQ ID NO: 248 - protein, 43 aa, synthetic construct
PSDPYLKEHLHWWVVTDIPGTTDATFGREVVSYEMPRXNIGIH

SEQ ID NO: 249 - DNA, 23 bp, synthetic construct
  source 1..23: mol_type = other DNA
gtagttggggtgaaggaatcaag

SEQ ID NO: 250 - DNA, 23 bp, synthetic construct
  source 1..23: mol_type = other DNA
gtggtgactgcagaaggtaagag

SEQ ID NO: 251 - DNA, 20 bp, synthetic construct
  source 1..20: mol_type = other DNA
ggcaccacagatgctacatt

SEQ ID NO: 252 - DNA, 5843 bp, synthetic construct
  source 1..5843: mol_type = other DNA
ccaccatatccaaatctctaaaccytaaaccytaaaatycaccatacacctaatcyctcgaccctarayycaaaatyyacyatacyyctaatytttcaaycctaarygycacaaatcattaaycaaaaaytmtaaaytctaaytcctcaaycwtaacyyttaaaytrtaaattytaaactcataaccttaaattytaaaccctaaatcctaaacgtaaatcccccaaaayyctarctacaartaattyctcaagtcttcytaattawtratyayrcyyaaattgggayatattacttratatcttctcaactargttgttatctactaacatgccttaycatttgyamtamatraatatatgaccayatyctttcctaaatycaaattaytaagcattatcaatttrctatwtagttgtctttrttttytcyatgwtgatraggtagtaacattgtcatstraatcraastttagtgaaagtaartgatttgaktygtttacaatcaattaaamaatagttctttcatgtggttataawyaatwrttccatrattgtaatctcaattaaarttcwtcwaaattrtacctcmtttcyaattaagtttacattgctaagtttttttttttataatatatatatatatatatatatattaaagtgcatgattctatttgattgataaaaaatatatttataaagataaaattataattgtaaggtaattcattrataaratrtttatatataaarctattaattataattgtaagtataattaattaacaagttttaaarttgagtaagcaatagccattgaatctgagatgagtatgatagactttgcataaaatgastaggcaattgtttaatgatgtgtatatgatgtggyatgccasatatgattgaracctcyacatcttaggycttagagcatytycaasagtttycytaaaaaatctttattatagaaattcaaaagttaaaatwtctawawtttttattttgcarttccaayagtttwtctatcatattctctataatttattatmtacaattaaatatggtattatttttaaatttwaaaartataatcaaataattaatratgraaatgaatatatttggaaagargaaagaraakagrgtaaaawtttyyaaaaasatrtttcyctataattttagagtttgctrtaaatatarakattyttattttctytcttctcactawctctaaaatagagatargtatagggaarytattggagyaaaaaaatactcaattwttcctaaactrgagaaaaaataaattatagggaarstcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccyyaatcycayatagcggtccatgtyatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaamgtattwttgctacaagaaaactawgtcatttttgaaaacaaaacctgkatttgtgaagatatcaagcacaatttwttwtwtcttttkgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccrtctttgatttgcctttacgactctctctctmkctctctctctctctctctctctctctctctckctmtctctctctctctcgtaagtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcwatgcttgtytcyrratttgaagaaaaytttagtttttttttttkrttacattggarggrwtgaacctytracctagyctaattctaaytcaattaytcaccaacccatactayttgtgcyaactctaagggmttggaaaacattagtttcttgaatatgtrtgtktatatatataaatatatatatatagatattaatgtgaatttgttttctyttcattacatggtyatttatgtttttggaattttagaatataattgratytagaaattaaagcaagaaaaaagtgcktatgaaaatctgaacwagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctstcyttcgcactcttcttctagtttaagtttagggttttggtktccwtttcatttsaacgggaaaatcakaaggaatcaacrcgatgacccaatctagtctgcgtgcgggccccacactgtcatgtccgatcttaaataactaattaacsgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgayaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttamaattcrttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctrtcggagatggagggtgcggtggtgatccgctaggctcaggagcagcagcggagagcartgatrggattrcaactgcggctckagcackggcycgakgacgacrycgrygrcrtgagatgggtgcagagagaagagaagaaaggaaaaggaaaagaagagaaattcggtgcccgaggcgaactgaaattttaaggcctaattttttctatataaatrtatacatttattttkrgtataatttttttcattaaatagaraaatgcgtaattattyaattaatcagartaatttgtgkagagtatgaaaaaatwtaagayatytttraaatyrtraaaattaatayttaagtwraattyttaaatytctwcattaattt

SEQ ID NO: 253 - protein, 172 aa, Rubus sp.
MARMSEPLVVGRVIGDVLDSFTPTTKMIVTYTTRHVSNGYELLPSAVTTKPRVEIQGGDMRSFFTLVMTDPDXPGPSDPYLKEHLHWVVTDIPGTTDATFGREVVSYEMPRPNIGIHRFVFVLFRQKRRQSVNPPSSRDHFNTRSFAAENDLGLPVAAVYFNAQRETAARRR

SEQ ID NO: 254 - DNA, 295 bp, synthetic construct; identical to SEQ ID NO: 217
  source 1..295: mol_type = other DNA

SEQ ID NO: 255 - DNA, 215 bp, synthetic construct; identical to SEQ ID NO: 218
  source 1..215: mol_type = other DNA

SEQ ID NO: 256 - DNA, 175 bp, synthetic construct; identical to SEQ ID NO: 219
  source 1..175: mol_type = other DNA

SEQ ID NO: 257 - DNA, 135 bp, synthetic construct; identical to SEQ ID NO: 220
  source 1..135: mol_type = other DNA

SEQ ID NO: 258 - DNA, 95 bp, synthetic construct; identical to SEQ ID NO: 221
  source 1..95: mol_type = other DNA

SEQ ID NO: 259 - DNA, 223 bp, synthetic construct; identical to SEQ ID NO: 222
  source 1..223: mol_type = other DNA

SEQ ID NO: 260 - DNA, 143 bp, synthetic construct; identical to SEQ ID NO: 223
  source 1..143: mol_type = other DNA

SEQ ID NO: 261 - DNA, 103 bp, synthetic construct; identical to SEQ ID NO: 224
  source 1..103: mol_type = other DNA

SEQ ID NO: 262 - DNA, 63 bp, synthetic construct; identical to SEQ ID NO: 225
  source 1..63: mol_type = other DNA

SEQ ID NO: 263 - DNA, 223 bp, synthetic construct; identical to SEQ ID NO: 226
  source 1..223: mol_type = other DNA

SEQ ID NO: 264 - DNA, 143 bp, synthetic construct; identical to SEQ ID NO: 242
  source 1..143: mol_type = other DNA

SEQ ID NO: 265 - DNA, 103 bp, synthetic construct; identical to SEQ ID NO: 228
  source 1..103: mol_type = other DNA

SEQ ID NO: 266 - DNA, 63 bp, synthetic construct; identical to SEQ ID NO: 229
  source 1..63: mol_type = other DNA

SEQ ID NO: 267 - DNA, 220 bp, synthetic construct
  source 1..220: mol_type = other DNA
aacwagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtatt

SEQ ID NO: 268 - DNA, 140 bp, synthetic construct
  source 1..140: mol_type = other DNA
agctgggarctaacagttttggtttaaattgaactaaacagggttgtgacagacattccaggcaccacagatgctacatttggttggttaattcactgctagttcagctaccagttatttagtgatcttatagctagaga

SEQ ID NO: 269 - DNA, 100 bp, synthetic construct; identical to SEQ ID NO: 245
  source 1..100: mol_type = other DNA

SEQ ID NO: 270 - DNA, 60 bp, synthetic construct; identical to SEQ ID NO: 246
  source 1..60: mol_type = other DNA

SEQ ID NO: 271 - protein, 41 aa, Rubus sp.
MARMSEPLVVGRVIGDVLDSFTPTTKMIVTYTTRHVSNGYE

SEQ ID NO: 272 - protein, 27 aa, Rubus sp.
LVVGRVIGDVLDSFTPTTKMIVTYTTR

SEQ ID NO: 273 - protein, 41 aa, Rubus sp.
TKMIVTYTTRHVSNGYELLPSAVTTKPRVEIQGGDMRSFFT

SEQ ID NO: 274 - protein, 27 aa, Rubus sp.
TTRHVSNGYELLPSAVTTKPRVEIQGG

SEQ ID NO: 275 - protein, 26 aa, Rubus sp.
HLHWVVTDIPGTTDATFGREVVSYEM

SEQ ID NO: 276 - protein, 26 aa, Rubus sp.
LHWWVVTDIPGTTDATFGREVVSYEM

SEQ ID NO: 277 - DNA, 5818 bp, synthetic construct
  source 1..5818: mol_type = other DNA
ccaccatatccaaatctctaaaccytaaaccytaaaatycaccatacacctaatccctcgaccctarayycaaaatyyacyataccyctaatytttcaaycctaaacgycacaaatcattaaycaaaaaytmtaaactctaaytcctcaaccttaacccttaaaytgtaaattytaaactcataaccttaaattytaaaccctaaatcctaaacgtaaatcccccaaaayyctagctacaagtaattyctcaagtcttcytaattawtgatyatgcyyaaattgggacatattacttgatatcttctcaactargttgttatctactaacatgccttatcatttgcactaaatraatatatgaccayattctttcctaaatycaaattactaagcattatcaatttrctatttagttgtctttrttttytcyatgatgatraggtagtaacattgtcatstraatcraastttagtgaaagtaartgatttgaktcgtttacaatcaattaaacaatagttctttcatgtggttataawcaatwrttccataattgtaatctcaattaaarttcatctaaattrtacctcctttcyaattaagtttacattgctaagtttttttttttataatatatatatatatatatatatattaaagtgcatgattctatttgattgataaaaaatatatttataaagataaaattataattgtaaggtaattcattrataaratrtttatatataaagctattaattataattgtaagtataattaattaacaagttttaaagttgagtaagcaatagccattgaatctgagatgagtatgatagactttgcataaaatgastaggcaattgtttaatgatgtgtatatgatgtggcatgccasatatgattgaracctcyacatcttaggtcttagagcatytycaasagttttcctaaaaaatctttattatagaaattcaaaagttaaaatwtctataatttttattttgcarttccaayagtttttctatcatattctctataatttattatctacaattaaatatggtattatttttaaatttaaaaagtataatcaaataattaatratgaaaatgaatatatttggaaagaggaaagagaakagggtaaaatttttyaaaaagatgtttcyctataattttagagtttgctrtaaatatagagattyttattttctctcttctcactatctctaaaatagagatargtatagggaarctattggagtaaaaaaatactcaatttttcctaaactrgagaaaaaataaattatagggaagctcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaacgtattwttgctacaagaaaactawgtcatttttgaaaacaaaacctgkatttgtgaagatatcaagcacaattttttttttctttttgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattgaccccaactaccaaaatgattgtcacttacactacgaggcacgtctctaatggatatgagctcttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctctcyttcgcactcttcttctagtttaagtttagggttttggtktccwtttcatttsaacgggaaaatcagaaggaatcaacrcgatgacccaatctagtctgcgtgcgggccccacactgtcatgtccgatcttaaataactaattaaccgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgacaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttaaaattcgttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctatcggagatggagggtgcggtggtgatccgctaggctcaggagcagcagcggagagcagtgatgggattrcaactgcggctcgagcackggcycgakgacgacrycgrygrcrtgagatgggtgcagagagaagagaagaaaggaaaaggaaaagaagagaaattcggtgcccgaggcgaactgaaattttaaggcctaattttttctatataaatgtatacatttatttttagtataatttttttcattaaatagagaaatgcgtaattattyaattaatcagagtaatttgtggagagtatgaaaaaatttaagacatytttgaaatcgtgaaaattaatacttaagttgaattcttaaatctcttcattaattt

SEQ ID NO: 278 - DNA, 5823 bp, synthetic construct
  source 1..5823: mol_type = other DNA
ccaccatatccaaatctctaaaccytaaaccytaaaatycaccatacacctaatccctcgaccctarayycaaaatyyacyataccyctaatytttcaaycctaaacgycacaaatcattaaycaaaaaytmtaaactctaaytcctcaaccttaacccttaaaytgtaaattytaaactcataaccttaaattytaaaccctaaatcctaaacgtaaatcccccaaaayyctagctacaagtaattyctcaagtcttcytaattawtgatyatgcyyaaattgggacatattacttgatatcttctcaactargttgttatctactaacatgccttatcatttgcactaaatraatatatgaccayattctttcctaaatycaaattactaagcattatcaatttrctatttagttgtctttrttttytcyatgatgatraggtagtaacattgtcatstraatcraastttagtgaaagtaartgatttgaktcgtttacaatcaattaaacaatagttctttcatgtggttataawcaatwrttccataattgtaatctcaattaaarttcatctaaattrtacctcctttcyaattaagtttacattgctaagtttttttttttataatatatatatatatatatatatattaaagtgcatgattctatttgattgataaaaaatatatttataaagataaaattataattgtaaggtaattcattrataaratrtttatatataaagctattaattataattgtaagtataattaattaacaagttttaaagttgagtaagcaatagccattgaatctgagatgagtatgatagactttgcataaaatgastaggcaattgtttaatgatgtgtatatgatgtggcatgccasatatgattgaracctcyacatcttaggtcttagagcatytycaasagttttcctaaaaaatctttattatagaaattcaaaagttaaaatwtctataatttttattttgcarttccaayagtttttctatcatattctctataatttattatctacaattaaatatggtattatttttaaatttaaaaagtataatcaaataattaatratgaaaatgaatatatttggaaagaggaaagagaakagggtaaaatttttyaaaaagatgtttcyctataattttagagtttgctrtaaatatagagattyttattttctctcttctcactatctctaaaatagagatargtatagggaarctattggagtaaaaaaatactcaatttttcctaaactrgagaaaaaataaattatagggaagctcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaacgtattwttgctacaagaaaactawgtcatttttgaaaacaaaacctgkatttgtgaagatatcaagcacaattttttttttctttttgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggattcaccccaactaccaaaatgattgtcacttacactacgaggcacgtctctaatggatatgagctcttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctctcyttcgcactcttcttctagtttaagtttagggttttggtktccwtttcatttsaacgggaaaatcagaaggaatcaacrcgatgacccaatctagtctgcgtgcgggccccacactgtcatgtccgatcttaaataactaattaaccgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgacaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttaaaattcgttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctatcggagatggagggtgcggtggtgatccgctaggctcaggagcagcagcggagagcagtgatgggattrcaactgcggctcgagcackggcycgakgacgacrycgrygrcrtgagatgggtgcagagagaagagaagaaaggaaaaggaaaagaagagaaattcggtgcccgaggcgaactgaaattttaaggcctaattttttctatataaatgtatacatttatttttagtataatttttttcattaaatagagaaatgcgtaattattyaattaatcagagtaatttgtggagagtatgaaaaaatttaagacatytttgaaatcgtgaaaattaatacttaagttgaattcttaaatctcttcattaattt

SEQ ID NO: 279 - DNA, 5826 bp, synthetic construct
  source 1..5826: mol_type = other DNA
ccaccatatccaaatctctaaaccytaaaccytaaaatycaccatacacctaatccctcgaccctarayycaaaatyyacyataccyctaatytttcaaycctaaacgycacaaatcattaaycaaaaaytmtaaactctaaytcctcaaccttaacccttaaaytgtaaattytaaactcataaccttaaattytaaaccctaaatcctaaacgtaaatcccccaaaayyctagctacaagtaattyctcaagtcttcytaattawtgatyatgcyyaaattgggacatattacttgatatcttctcaactargttgttatctactaacatgccttatcatttgcactaaatraatatatgaccayattctttcctaaatycaaattactaagcattatcaatttrctatttagttgtctttrttttytcyatgatgatraggtagtaacattgtcatstraatcraastttagtgaaagtaartgatttgaktcgtttacaatcaattaaacaatagttctttcatgtggttataawcaatwrttccataattgtaatctcaattaaarttcatctaaattrtacctcctttcyaattaagtttacattgctaagtttttttttttataatatatatatatatatatatatattaaagtgcatgattctatttgattgataaaaaatatatttataaagataaaattataattgtaaggtaattcattrataaratrtttatatataaagctattaattataattgtaagtataattaattaacaagttttaaagttgagtaagcaatagccattgaatctgagatgagtatgatagactttgcataaaatgastaggcaattgtttaatgatgtgtatatgatgtggcatgccasatatgattgaracctcyacatcttaggtcttagagcatytycaasagttttcctaaaaaatctttattatagaaattcaaaagttaaaatwtctataatttttattttgcarttccaayagtttttctatcatattctctataatttattatctacaattaaatatggtattatttttaaatttaaaaagtataatcaaataattaatratgaaaatgaatatatttggaaagaggaaagagaakagggtaaaatttttyaaaaagatgtttcyctataattttagagtttgctrtaaatatagagattyttattttctctcttctcactatctctaaaatagagatargtatagggaarctattggagtaaaaaaatactcaatttttcctaaactrgagaaaaaataaattatagggaagctcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaacgtattwttgctacaagaaaactawgtcatttttgaaaacaaaacctgkatttgtgaagatatcaagcacaattttttttttctttttgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttttcaccccaactaccaaaatgattgtcacttacactacgaggcacgtctctaatggatatgagctctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctctcyttcgcactcttcttctagtttaagtttagggttttggtktccwtttcatttsaacgggaaaatcagaaggaatcaacrcgatgacccaatctagtctgcgtgcgggccccacactgtcatgtccgatcttaaataactaattaaccgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgacaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttaaaattcgttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctatcggagatggagggtgcggtggtgatccgctaggctcaggagcagcagcggagagcagtgatgggattrcaactgcggctcgagcackggcycgakgacgacrycgrygrcrtgagatgggtgcagagagaagagaagaaaggaaaaggaaaagaagagaaattcggtgcccgaggcgaactgaaattttaaggcctaattttttctatataaatgtatacatttatttttagtataatttttttcattaaatagagaaatgcgtaattattyaattaatcagagtaatttgtggagagtatgaaaaaatttaagacatytttgaaatcgtgaaaattaatacttaagttgaattcttaaatctcttcattaattt

SEQ ID NO: 280 - DNA, 5764 bp, synthetic construct
  source 1..5764: mol_type = other DNA
ccaccatatccaaatctctaaaccytaaaccytaaaatycaccatacacctaatccctcgaccctarayycaaaatyyacyataccyctaatytttcaaycctaaacgycacaaatcattaaycaaaaaytmtaaactctaaytcctcaaccttaacccttaaaytgtaaattytaaactcataaccttaaattytaaaccctaaatcctaaacgtaaatcccccaaaayyctagctacaagtaattyctcaagtcttcytaattawtgatyatgcyyaaattgggacatattacttgatatcttctcaactargttgttatctactaacatgccttatcatttgcactaaatraatatatgaccayattctttcctaaatycaaattactaagcattatcaatttrctatttagttgtctttrttttytcyatgatgatraggtagtaacattgtcatstraatcraastttagtgaaagtaartgatttgaktcgtttacaatcaattaaacaatagttctttcatgtggttataawcaatwrttccataattgtaatctcaattaaarttcatctaaattrtacctcctttcyaattaagtttacattgctaagtttttttttttataatatatatatatatatatatatattaaagtgcatgattctatttgattgataaaaaatatatttataaagataaaattataattgtaaggtaattcattrataaratrtttatatataaagctattaattataattgtaagtataattaattaacaagttttaaagttgagtaagcaatagccattgaatctgagatgagtatgatagactttgcataaaatgastaggcaattgtttaatgatgtgtatatgatgtggcatgccasatatgattgaracctcyacatcttaggtcttagagcatytycaasagttttcctaaaaaatctttattatagaaattcaaaagttaaaatwtctataatttttattttgcarttccaayagtttttctatcatattctctataatttattatctacaattaaatatggtattatttttaaatttaaaaagtataatcaaataattaatratgaaaatgaatatatttggaaagaggaaagagaakagggtaaaatttttyaaaaagatgtttcyctataattttagagtttgctrtaaatatagagattyttattttctctcttctcactatctctaaaatagagatargtatagggaarctattggagtaaaaaaatactcaatttttcctaaactrgagaaaaaataaattatagggaagctcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaacgtattwttgctacaagaaaactawgtcatttttgaaaacaaaacctgkatttgtgaagatatcaagcacaattttttttttctttttgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttcttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctctcyttcgcactcttcttctagtttaagtttagggttttggtktccwtttcatttsaacgggaaaatcagaaggaatcaacrcgatgacccaatctagtctgcgtgcgggccccacactgtcatgtccgatcttaaataactaattaaccgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgacaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttaaaattcgttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctatcggagatggagggtgcggtggtgatccgctaggctcaggagcagcagcggagagcagtgatgggattrcaactgcggctcgagcackggcycgakgacgacrycgrygrcrtgagatgggtgcagagagaagagaagaaaggaaaaggaaaagaagagaaattcggtgcccgaggcgaactgaaattttaaggcctaattttttctatataaatgtatacatttatttttagtataatttttttcattaaatagagaaatgcgtaattattyaattaatcagagtaatttgtggagagtatgaaaaaatttaagacatytttgaaatcgtgaaaattaatacttaagttgaattcttaaatctcttcattaattt

SEQ ID NO: 281 - DNA, 5820 bp, synthetic construct
  source 1..5820: mol_type = other DNA
ccaccatatccaaatctctaaaccytaaaccytaaaatycaccatacacctaatccctcgaccctarayycaaaatyyacyataccyctaatytttcaaycctaaacgycacaaatcattaaycaaaaaytmtaaactctaaytcctcaaccttaacccttaaaytgtaaattytaaactcataaccttaaattytaaaccctaaatcctaaacgtaaatcccccaaaayyctagctacaagtaattyctcaagtcttcytaattawtgatyatgcyyaaattgggacatattacttgatatcttctcaactargttgttatctactaacatgccttatcatttgcactaaatraatatatgaccayattctttcctaaatycaaattactaagcattatcaatttrctatttagttgtctttrttttytcyatgatgatraggtagtaacattgtcatstraatcraastttagtgaaagtaartgatttgaktcgtttacaatcaattaaacaatagttctttcatgtggttataawcaatwrttccataattgtaatctcaattaaarttcatctaaattrtacctcctttcyaattaagtttacattgctaagtttttttttttataatatatatatatatatatatatattaaagtgcatgattctatttgattgataaaaaatatatttataaagataaaattataattgtaaggtaattcattrataaratrtttatatataaagctattaattataattgtaagtataattaattaacaagttttaaagttgagtaagcaatagccattgaatctgagatgagtatgatagactttgcataaaatgastaggcaattgtttaatgatgtgtatatgatgtggcatgccasatatgattgaracctcyacatcttaggtcttagagcatytycaasagttttcctaaaaaatctttattatagaaattcaaaagttaaaatwtctataatttttattttgcarttccaayagtttttctatcatattctctataatttattatctacaattaaatatggtattatttttaaatttaaaaagtataatcaaataattaatratgaaaatgaatatatttggaaagaggaaagagaakagggtaaaatttttyaaaaagatgtttcyctataattttagagtttgctrtaaatatagagattyttattttctctcttctcactatctctaaaatagagatargtatagggaarctattggagtaaaaaaatactcaatttttcctaaactrgagaaaaaataaattatagggaagctcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaacgtattwttgctacaagaaaactawgtcatttttgaaaacaaaacctgkatttgtgaagatatcaagcacaattttttttttctttttgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggttcaccccaactaccaaaatgattgtcacttacactacgaggcacgtctctaatggatatgagcttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctctcyttcgcactcttcttctagtttaagtttagggttttggtktccwtttcatttsaacgggaaaatcagaaggaatcaacrcgatgacccaatctagtctgcgtgcgggccccacactgtcatgtccgatcttaaataactaattaaccgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgacaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttaaaattcgttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctatcggagatggagggtgcggtggtgatccgctaggctcaggagcagcagcggagagcagtgatgggattrcaactgcggctcgagcackggcycgakgacgacrycgrygrcrtgagatgggtgcagagagaagagaagaaaggaaaaggaaaagaagagaaattcggtgcccgaggcgaactgaaattttaaggcctaattttttctatataaatgtatacatttatttttagtataatttttttcattaaatagagaaatgcgtaattattyaattaatcagagtaatttgtggagagtatgaaaaaatttaagacatytttgaaatcgtgaaaattaatacttaagttgaattcttaaatctcttcattaattt

SEQ ID NO: 282 - DNA, 5822 bp, synthetic construct
  source 1..5822: mol_type = other DNA
ccaccatatccaaatctctaaaccytaaaccytaaaatycaccatacacctaatccctcgaccctarayycaaaatyyacyataccyctaatytttcaaycctaaacgycacaaatcattaaycaaaaaytmtaaactctaaytcctcaaccttaacccttaaaytgtaaattytaaactcataaccttaaattytaaaccctaaatcctaaacgtaaatcccccaaaayyctagctacaagtaattyctcaagtcttcytaattawtgatyatgcyyaaattgggacatattacttgatatcttctcaactargttgttatctactaacatgccttatcatttgcactaaatraatatatgaccayattctttcctaaatycaaattactaagcattatcaatttrctatttagttgtctttrttttytcyatgatgatraggtagtaacattgtcatstraatcraastttagtgaaagtaartgatttgaktcgtttacaatcaattaaacaatagttctttcatgtggttataawcaatwrttccataattgtaatctcaattaaarttcatctaaattrtacctcctttcyaattaagtttacattgctaagtttttttttttataatatatatatatatatatatatattaaagtgcatgattctatttgattgataaaaaatatatttataaagataaaattataattgtaaggtaattcattrataaratrtttatatataaagctattaattataattgtaagtataattaattaacaagttttaaagttgagtaagcaatagccattgaatctgagatgagtatgatagactttgcataaaatgastaggcaattgtttaatgatgtgtatatgatgtggcatgccasatatgattgaracctcyacatcttaggtcttagagcatytycaasagttttcctaaaaaatctttattatagaaattcaaaagttaaaatwtctataatttttattttgcarttccaayagtttttctatcatattctctataatttattatctacaattaaatatggtattatttttaaatttaaaaagtataatcaaataattaatratgaaaatgaatatatttggaaagaggaaagagaakagggtaaaatttttyaaaaagatgtttcyctataattttagagtttgctrtaaatatagagattyttattttctctcttctcactatctctaaaatagagatargtatagggaarctattggagtaaaaaaatactcaatttttcctaaactrgagaaaaaataaattatagggaagctcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaacgtattwttgctacaagaaaactawgtcatttttgaaaacaaaacctgkatttgtgaagatatcaagcacaattttttttttctttttgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggttcaccccaactaccaaaatgattgtcacttacactacgaggcacgtctctaatggatatgagctcttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctctcyttcgcactcttcttctagtttaagtttagggttttggtktccwtttcatttsaacgggaaaatcagaaggaatcaacrcgatgacccaatctagtctgcgtgcgggccccacactgtcatgtccgatcttaaataactaattaaccgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgacaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttaaaattcgttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctatcggagatggagggtgcggtggtgatccgctaggctcaggagcagcagcggagagcagtgatgggattrcaactgcggctcgagcackggcycgakgacgacrycgrygrcrtgagatgggtgcagagagaagagaagaaaggaaaaggaaaagaagagaaattcggtgcccgaggcgaactgaaattttaaggcctaattttttctatataaatgtatacatttatttttagtataatttttttcattaaatagagaaatgcgtaattattyaattaatcagagtaatttgtggagagtatgaaaaaatttaagacatytttgaaatcgtgaaaattaatacttaagttgaattcttaaatctcttcattaattt

SEQ ID NO: 283 - DNA, 5756 bp, synthetic construct
  source 1..5756: mol_type = other DNA
ccaccatatccaaatctctaaaccytaaaccytaaaatycaccatacacctaatccctcgaccctarayycaaaatyyacyataccyctaatytttcaaycctaaacgycacaaatcattaaycaaaaaytmtaaactctaaytcctcaaccttaacccttaaaytgtaaattytaaactcataaccttaaattytaaaccctaaatcctaaacgtaaatcccccaaaayyctagctacaagtaattyctcaagtcttcytaattawtgatyatgcyyaaattgggacatattacttgatatcttctcaactargttgttatctactaacatgccttatcatttgcactaaatraatatatgaccayattctttcctaaatycaaattactaagcattatcaatttrctatttagttgtctttrttttytcyatgatgatraggtagtaacattgtcatstraatcraastttagtgaaagtaartgatttgaktcgtttacaatcaattaaacaatagttctttcatgtggttataawcaatwrttccataattgtaatctcaattaaarttcatctaaattrtacctcctttcyaattaagtttacattgctaagtttttttttttataatatatatatatatatatatatattaaagtgcatgattctatttgattgataaaaaatatatttataaagataaaattataattgtaaggtaattcattrataaratrtttatatataaagctattaattataattgtaagtataattaattaacaagttttaaagttgagtaagcaatagccattgaatctgagatgagtatgatagactttgcataaaatgastaggcaattgtttaatgatgtgtatatgatgtggcatgccasatatgattgaracctcyacatcttaggtcttagagcatytycaasagttttcctaaaaaatctttattatagaaattcaaaagttaaaatwtctataatttttattttgcarttccaayagtttttctatcatattctctataatttattatctacaattaaatatggtattatttttaaatttaaaaagtataatcaaataattaatratgaaaatgaatatatttggaaagaggaaagagaakagggtaaaatttttyaaaaagatgtttcyctataattttagagtttgctrtaaatatagagattyttattttctctcttctcactatctctaaaatagagatargtatagggaarctattggagtaaaaaaatactcaatttttcctaaactrgagaaaaaataaattatagggaagctcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaacgtattwttgctacaagaaaactawgtcatttttgaaaacaaaacctgkatttgtgaagatatcaagcacaattttttttttctttttgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctctcyttcgcactcttcttctagtttaagtttagggttttggtktccwtttcatttsaacgggaaaatcagaaggaatcaacrcgatgacccaatctagtctgcgtgcgggccccacactgtcatgtccgatcttaaataactaattaaccgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgacaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttaaaattcgttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctatcggagatggagggtgcggtggtgatccgctaggctcaggagcagcagcggagagcagtgatgggattrcaactgcggctcgagcackggcycgakgacgacrycgrygrcrtgagatgggtgcagagagaagagaagaaaggaaaaggaaaagaagagaaattcggtgcccgaggcgaactgaaattttaaggcctaattttttctatataaatgtatacatttatttttagtataatttttttcattaaatagagaaatgcgtaattattyaattaatcagagtaatttgtggagagtatgaaaaaatttaagacatytttgaaatcgtgaaaattaatacttaagttgaattcttaaatctcttcattaattt

SEQ ID NO: 284 - DNA, 5828 bp, synthetic construct
  source 1..5828: mol_type = other DNA
ccaccatatccaaatctctaaaccytaaaccytaaaatycaccatacacctaatccctcgaccctarayycaaaatyyacyataccyctaatytttcaaycctaaacgycacaaatcattaaycaaaaaytmtaaactctaaytcctcaaccttaacccttaaaytgtaaattytaaactcataaccttaaattytaaaccctaaatcctaaacgtaaatcccccaaaayyctagctacaagtaattyctcaagtcttcytaattawtgatyatgcyyaaattgggacatattacttgatatcttctcaactargttgttatctactaacatgccttatcatttgcactaaatraatatatgaccayattctttcctaaatycaaattactaagcattatcaatttrctatttagttgtctttrttttytcyatgatgatraggtagtaacattgtcatstraatcraastttagtgaaagtaartgatttgaktcgtttacaatcaattaaacaatagttctttcatgtggttataawcaatwrttccataattgtaatctcaattaaarttcatctaaattrtacctcctttcyaattaagtttacattgctaagtttttttttttataatatatatatatatatatatatattaaagtgcatgattctatttgattgataaaaaatatatttataaagataaaattataattgtaaggtaattcattrataaratrtttatatataaagctattaattataattgtaagtataattaattaacaagttttaaagttgagtaagcaatagccattgaatctgagatgagtatgatagactttgcataaaatgastaggcaattgtttaatgatgtgtatatgatgtggcatgccasatatgattgaracctcyacatcttaggtcttagagcatytycaasagttttcctaaaaaatctttattatagaaattcaaaagttaaaatwtctataatttttattttgcarttccaayagtttttctatcatattctctataatttattatctacaattaaatatggtattatttttaaatttaaaaagtataatcaaataattaatratgaaaatgaatatatttggaaagaggaaagagaakagggtaaaatttttyaaaaagatgtttcyctataattttagagtttgctrtaaatatagagattyttattttctctcttctcactatctctaaaatagagatargtatagggaarctattggagtaaaaaaatactcaatttttcctaaactrgagaaaaaataaattatagggaagctcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaacgtattwttgctacaagaaaactawgtcatttttgaaaacaaaacctgkatttgtgaagatatcaagcacaattttttttttctttttgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttttcaccccaactaccaaaatgattgtcacttacactacgaggcacgtctctaatggatatgagccttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctctcyttcgcactcttcttctagtttaagtttagggttttggtktccwtttcatttsaacgggaaaatcagaaggaatcaacrcgatgacccaatctagtctgcgtgcgggccccacactgtcatgtccgatcttaaataactaattaaccgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgacaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttaaaattcgttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctatcggagatggagggtgcggtggtgatccgctaggctcaggagcagcagcggagagcagtgatgggattrcaactgcggctcgagcackggcycgakgacgacrycgrygrcrtgagatgggtgcagagagaagagaagaaaggaaaaggaaaagaagagaaattcggtgcccgaggcgaactgaaattttaaggcctaattttttctatataaatgtatacatttatttttagtataatttttttcattaaatagagaaatgcgtaattattyaattaatcagagtaatttgtggagagtatgaaaaaatttaagacatytttgaaatcgtgaaaattaatacttaagttgaattcttaaatctcttcattaattt

SEQ ID NO: 285 - protein, 30 aa, synthetic construct
MARMSEPLVVGRVIDPNYQNDCHLHYEARL

SEQ ID NO: 286 - protein, 44 aa, synthetic construct
MARMSEPLVVGRVIGFTPTTKMIVTYTTRHVSNGYELFCSHHQT

SEQ ID NO: 287 - protein, 45 aa, synthetic construct
MARMSEPLVVGRVIGDVFTPTTKMIVTYTTRHVSNGYELCSHHQT

SEQ ID NO: 288 - protein, 33 aa, synthetic construct
MARMSEPLVVGRVIGDVLLQSPPNLELRFKEVT

SEQ ID NO: 289 - protein, 21 aa, synthetic construct
MARMSEPLVVGRVIGSPQLPK

SEQ ID NO: 290 - protein, 143 aa, synthetic construct
MARMSEPLVVGRVIGSAVTTKPRVEIQGGDMRSFFTLVMTDPDXPGPSDPYLKEHLHWVVTDIPGTTDATFGREVVSYEMPRPNIGIHRFVFVLFRQKRRQSVNPPSSRDHFNTRSFAAENDLGLPVAAVYFNAQRETAARRR

SEQ ID NO: 291 - protein, 167 aa, synthetic construct
MARMSEPLVVGRVIGDVFTPTTKMIVTYTTRHVSNGYEPSAVTTKPRVEIQGGDMRSFFTLVMTDPDXPGPSDPYLKEHLHWVVTDIPGTTDATFGREVVSYEMPRPNIGIHRFVFVLFRQKRRQSVNPPSSRDHFNTRSFAAENDLGLPVAAVYFNAQRETAARRR

SEQ ID NO: 292 - DNA, 16 bp, synthetic construct
  source 1..16: mol_type = other DNA
agatgttcttgattcc

SEQ ID NO: 293 - DNA, 87 bp, synthetic construct
  source 1..87: mol_type = other DNA
agatgttcttgattccttcaccccaactaccaaaatgattgtcacttacactacgaggcacgtctctaatggatatgagctcttacc

SEQ ID NO: 294 - DNA, 16 bp, synthetic construct
  source 1..16: mol_type = other DNA
agatgttcttgattcc

SEQ ID NO: 295 - DNA, 1778 bp, synthetic construct
  source 1..1778: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgtgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 296 - protein, 31 aa, synthetic construct
MARMSEPLVVGRVIGDVQSPPNLELRFKEVT

SEQ ID NO: 297 - DNA, 1784 bp, synthetic construct
  source 1..1784: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttcttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 298 - DNA, 1851 bp, synthetic construct
  source 1..1851: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttctcttcaccccaactaccaaaatgattgtcacttacactacgaggcacgtctctaatggatatgagccttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 299 - protein, 168 aa, synthetic construct
MARMSEPLVVGRVIGDVLFTPTTKMIVTYTTRHVSNGYEPSAVTTKPRVEIQGGDMRSFFTLVMTDPDXPGPSDPYLKEHLHWVVTDIPGTTDATFGREVVSYEMPRPNIGIHRFVFVLFRQKRRQSVNPPSSRDHFNTRSFAAENDLGLPVAAVYFNAQRETAARRR

SEQ ID NO: 300 - DNA, 1839 bp, synthetic construct
  source 1..1839: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggattcaccccaactaccaaaatgattgtcacttacactacgaggcacgtctctaatggatatgagtctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 301 - protein, 164 aa, synthetic construct
MARMSEPLVVGRVIGFTPTTKMIVTYTTRHVSNGYESAVTTKPRVEIQGGDMRSFFTLVMTDPDXPGPSDPYLKEHLHWVVTDIPGTTDATFGREVVSYEMPRPNIGIHRFVFVLFRQKRRQSVNPPSSRDHFNTRSFAAENDLGLPVAAVYFNAQRETAARRR

SEQ ID NO: 302 - DNA, 1845 bp, synthetic construct
  source 1..1845: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagcttcaccccaactaccaaaatgattgtcacttacactacgaggcacgtctctaatggatatgagctcttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 303 - protein, 22 aa, synthetic construct
MARMSEPLVVGRVIGASPQLPK

SEQ ID NO: 304 - DNA, 1847 bp, synthetic construct
  source 1..1847: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttcaccccaactaccaaaatgattgtcacttacactacgaggcacgtctctaatggatatgagctcttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 305 - protein, 33 aa, synthetic construct
MARMSEPLVVGRVIGDVHPNYQNDCHLHYEARL

SEQ ID NO: 306 - DNA, 1786 bp, synthetic construct
  source 1..1786: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttcccttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 307 - protein, 25 aa, synthetic construct
MARMSEPLVVGRVIGDVPFCSHHQT

SEQ ID NO: 308 - DNA, 1838 bp, synthetic construct
  source 1..1838: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattgaccccaactaccaaaatgattgtcacttacactacgaggcacgtctctaatggatatgagctcttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 309 - DNA, 1843 bp, synthetic construct
  source 1..1843: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggattcaccccaactaccaaaatgattgtcacttacactacgaggcacgtctctaatggatatgagctcttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 310 - DNA, 1846 bp, synthetic construct
  source 1..1846: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttttcaccccaactaccaaaatgattgtcacttacactacgaggcacgtctctaatggatatgagctctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 311 - DNA, 1784 bp, synthetic construct; identical to SEQ ID NO: 297
  source 1..1784: mol_type = other DNA

SEQ ID NO: 312 - protein, 34 aa, synthetic construct
MARMSEPLVVGRVIGDVLHPNYQNDCHLHYEARL

SEQ ID NO: 313 - DNA, 1848 bp, synthetic construct
  source 1..1848: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttcttcaccccaactaccaaaatgattgtcacttacactacgaggcacgtctctaatggatatgagctcctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

(19) United States
(12) Patent Application Publication
(10) Pub. No.: US 2026/0043040 A1
(43) Pub. Date: Feb. 12, 2026

(54) EARLY FLOWERING ROSACEAE PLANTS WITH IMPROVED CHARACTERISTICS

(71) Applicant: PAIRWISE PLANTS SERVICES, INC., Durham, NC (US)

(72) Inventors: Brian Charles Wilding Crawford, Cary, NC (US); Nicholas Bate, Raleigh, NC (US)

(21) Appl. No.: 18/851,097

(22) PCT Filed: Mar. 28, 2023
(86) PCT No.: PCT/US2023/065013

§ 371 Date: Sep. 26, 2024

Related U.S. Application Data
(60) Provisional application No. 63/325,815, filed on Mar. 31, 2022.

(51) Int. Cl. C12N 15/82

(52) U.S. Cl. CPC C12N 15/827

(57) ABSTRACT

This invention relates to compositions and methods for modifying TFL genes in Rosaceae plants to remove or reduce the dependency on environmental triggers for flowering, optionally producing plants having improved characteristics for breeding and production, optionally, including a reduced time to initiate flowering, a longer duration of flowering, and/or improved yield characteristics. The invention further relates to Rosaceae plants produced using the methods and compositions of the invention.

### STATEMENT OF PRIORITY

This application claims the benefit, under 35 U.S.C. § 119 (e), of U.S. Provisional Application No. 63/325,815 filed on Mar. 31, 2022, the entire contents of which is incorporated by reference herein.

### STATEMENT REGARDING ELECTRONIC FILING OF A SEQUENCE LISTING

A Sequence Listing in XML format, entitled 1499_93WO_ST26.xml, 521,889 bytes in size, generated on Mar. 23, 2023 and filed herewith, is hereby incorporated by reference into the specification for its disclosures

### FIELD OF THE INVENTION

This invention relates to compositions and methods for modifying TFL genes in Rosaceae plants to remove or reduce the dependency on environmental triggers for flowering, optionally producing plants having improved characteristics for breeding and production, optionally, including a reduced time to initiate flowering, a longer duration of flowering, and/or improved yield characteristics. The invention further relates to Rosaceae plants produced using the methods and compositions of the invention.

### BACKGROUND OF THE INVENTION

Genetic gain through breeding in Rosaceae is limited by environmental controls that are required to trigger flowering and a long juvenile phase, often several years long. Natural variants in flowering time genes have reduced environmental dependency for flowering as well as altered plant architecture and improved determinate growth habit to generate variants that are more conducive to plant production. However, such natural variation is relatively rare and has occurred in only a few plant species.

The present invention overcomes these shortcomings by providing methods for modifying the flowering time in Rosaceae plants.

### SUMMARY OF THE INVENTION

One aspect of the invention provides a Rosaceae plant or part thereof comprising at least one mutation in an endogenous TERMINAL FLOWER (TFL) gene encoding a TFL protein, optionally wherein the at least one mutation may be a non-natural mutation.

A second aspect of the invention provides a Rosaceae plant cell, comprising an editing system comprising: (a) a CRISPR-Cas effector protein; and (b) a guide nucleic acid having a spacer sequence with complementarity to (a portion of) an endogenous target gene encoding a TFL protein in the Rosaceae plant cell.

A third aspect of the invention provides Rosaceae plant cell comprising at least one 1 mutation within an endogenous TFL gene, wherein the at least one mutation is a base substitution, base insertion or a base deletion that is introduced using an editing system that comprises a nucleic acid binding domain that binds to a target site in the endogenous TFL gene, optionally wherein the at least one mutation may be a non-natural mutation.

A fourth aspect of the invention provides a method of producing/breeding a transgene-free edited Rosaceae plant, comprising: crossing the Rosaceae plant of the invention with a transgene free Rosaceae plant, thereby introducing the at least mutation into the Rosaceae plant that is transgene-free; and selecting a progeny Rosaceae plant that comprises the at least one mutation and is transgene-free, thereby producing a transgene free edited Rosaceae plant.

In a fifth aspect, a method of providing a plurality of Rosaceae plants having a reduced time to flowering (less time for flower initiation), a longer duration of flowering, and/or one or more improved yield characteristics is provided, the method comprising planting two or more Rosaceae plants of the invention in a growing area, thereby providing a plurality of Rosaceae plants having reduced time to flowering (less time for flower initiation), longer duration of flowering, and/or one or more improved yield characteristics as compared to a plurality of control Rosaceae plants not comprising the mutation.

A sixth aspect of the invention provides a method for editing a specific site in the genome of a Rosaceae plant cell, the method comprising: cleaving, in a site-specific manner, a target site within an endogenous TFL gene in the Rosaceae plant cell, wherein the endogenous TFL gene (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, thereby generating an edit in the endogenous TFL gene of the Rosaceae plant cell and producing a plant cell comprising the edit in the endogenous TFL gene.

A seventh aspect of the invention provides a method for making a Rosaceae plant, comprising: (a) contacting a population of Rosaceae plant cells comprising at least one endogenous TFL gene with a nuclease linked to a nucleic acid binding domain (e.g., an editing system) that binds to a target site in the at least one endogenous TFL gene, wherein the at least one endogenous TFL gene: (i) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (ii) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (iii) encodes an amino acid sequence having at least 80% sequence identity to any one of SEQ ID NOs:74, 108, 116, 128, 142, 154, 164, 174, 186, 216, 236 or 253, and/or (iv) encodes a region having at least 80% sequence identity to any one of the amino acid sequences of SEQ ID NOs:193-197, 247, 248, or 271-276; (b) selecting a Rosaceae plant cell from said population that comprises a mutation in the at least one endogenous TFL gene, optionally wherein the mutation may be a non-natural mutation; and (c) growing the selected Rosaceae plant cell into a Rosaceae plant.

An eighth aspect of the invention provides a method for reducing time to flowering in a Rosaceae plant or part thereof, comprising (a) contacting a Rosaceae plant cell comprising an endogenous TFL gene with a nuclease targeting the endogenous TFL gene, wherein the nuclease is linked to a nucleic acid binding domain that binds to a target site in the endogenous TFL gene, wherein the endogenous TFL gene: (i) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (ii) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (iii) encodes an amino acid sequence having at least 80% sequence identity to any one of SEQ ID NOs:74, 108, 116, 128, 142, 154, 164, 174, 186, 216, 236 or 253, and/or (iv) encodes a region having at least 80% sequence identity to any one of the amino acid sequences of SEQ ID NOs:193-197, 247, 248, or 271-276; and (b) growing the Rosaceae plant cell into a Rosaceae plant comprising a mutation in the endogenous TFL gene, optionally wherein the mutation may be a non-natural mutation, thereby reducing time to flowering in a Rosaceae plant or part thereof.

A ninth aspect provides a method for producing a Rosaceae plant or part thereof comprising at least one cell having a mutated endogenous TFL gene, the method comprising contacting a target site in an endogenous TFL gene in the Rosaceae plant or part with a nuclease comprising a cleavage domain and a DNA-binding domain, wherein the nucleic acid binding domain binds to a target site in the endogenous TFL gene, wherein the endogenous TFL gene (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, thereby producing the Rosaceae plant or part thereof comprising at least one cell having an endogenous TFL gene with a mutation.

A tenth aspect provides a method for producing a Rosaceae plant or part thereof comprising a mutated endogenous TFL gene and exhibiting reduced time to flowering, a longer duration of flowering, one or more improved yield characteristics and/or a more determinate plant growth pattern, the method comprising contacting a target site in an endogenous TFL gene in the Rosaceae plant or part thereof with a nuclease comprising a cleavage domain and a DNA-binding domain, wherein the nucleic acid binding domain binds to a target site in the endogenous TFL gene, wherein the endogenous TFL gene: (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, thereby producing the Rosaceae plant or part thereof comprising an endogenous TFL gene having a mutation and reduced time to flowering, longer duration of flowering, one or more improved yield characteristics and/or a more determinate plant growth pattern.

An eleventh aspect provides a guide nucleic acid that binds to a target site in a TFL gene, wherein the target site is in a region of the TFL gene having at least 80% sequence identity to any one of SEQ ID NOs:75-105, 109-113, 117-125, 129-139, 143-152, 155-161, 165-171, 175-183, 187-192, 217-233, 237-246, or 254-270.

In a twelfth aspect a system is provided comprising a guide nucleic acid of the invention and a CRISPR-Cas effector protein that associates with the guide nucleic acid.

In a thirteenth aspect, a gene editing system is provided comprising a CRISPR-Cas effector protein in association with a guide nucleic acid, wherein the guide nucleic acid comprises a spacer sequence that binds to a TFL gene.

A fourteenth aspect provides a complex comprising a CRISPR-Cas effector protein having a cleavage domain and a guide nucleic acid (e.g., gRNA), wherein the guide nucleic acid binds to a target site in a TFL gene, wherein the TFL gene (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, wherein the cleavage domain cleaves a target strand in the TFL gene.

In a fifteenth aspect, an expression cassette is provided comprising (a) a polynucleotide encoding CRISPR-Cas effector protein comprising a cleavage domain and (b) a guide nucleic acid that binds to a target site in an endogenous TFL gene, wherein the guide nucleic acid comprises a spacer sequence that is complementary to and binds to (i) a portion of nucleic acid having at least 80% sequence identity to any one of the nucleotide sequences of SEQ ID NOs:72, 73, 106, 107, 114, 115, 126, 127, 140, 141, 153, 162, 163, 172, 173, 184, 185, 214, 215, 234, 235, or 252; (ii) a portion of nucleic acid having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270; (iii) a portion of a nucleic acid encoding an amino acid sequence having at least 80% sequence identity to any one of the amino acid sequences of SEQ ID NOs:74, 108, 116, 128, 142, 154, 164, 174, 186, 216, 236 or 253; and/or (iv) a portion of a nucleic acid encoding an amino acid sequence having at least 80% sequence identity to any one of SEQ ID NOs:193-197, 247, 248, or 271-276.

A sixteenth aspect of the invention provides a mutated endogenous TFL gene, wherein the mutated endogenous TFL gene comprises a nucleic acid sequence having a at least 90% sequence identity to any one of SEQ ID NO:277-284, 295, 297, 298, 300, 302, 304, 306, 308, 309, 310, 311, or 313 and/or encodes a mutated TFL1 polypeptide having at least 90% sequence identity to any one of SEQ ID NOs:285-291, 296, 299, 301, 303, 305, 307, or 312, optionally wherein the mutated endogenous TFL gene comprises a non-natural mutation.

In a seventeenth aspect, a method is provided for creating a mutation in an endogenous TERMINAL FLOWER (TFL) gene in a plant, the method comprising: (a) targeting a gene editing system to a portion of the TFL gene that comprises a sequence having at least 90% sequence identity to any one of SEQ ID NOs:75-105, 109-113, 117-125, 129-139, 143-152, 155-161, 165-171, 175-183, 187-192, 217-233, 237-246, or 254-270; and (b) selecting a plant that comprises a modification located in a region of the one or more TFL genes having at least 90% identity to any one of SEQ ID NOs:75-105, 109-113, 117-125, 129-139, 143-152, 155-161, 165-171, 175-183, 187-192, 217-233, 237-246, or 254-270, optionally wherein the mutated endogenous TFL gene comprises a non-natural mutation.

An eighteenth aspect provides a nucleic acid encoding a null mutation or a dominant negative mutation of a Rosaceae TFL gene and/or a Rosaceae plant or part thereof comprising the nucleic acid encoding a null mutation or a dominant negative mutation of a Rosaceae TFL gene, optionally wherein the Rosaceae plant exhibits a reduced time to flowering, a longer duration of flowering, one or more improved yield characteristics and/or a more determinate plant growth pattern when compared to a control plant.

In a nineteenth aspect, a mutated endogenous TFL gene is provided that is produced by contacting a target site in an endogenous TFL gene in a Rosaceae plant or part thereof with a nuclease comprising a cleavage domain and a nucleic acid binding domain, wherein the nucleic acid binding domain binds to a target site in the endogenous TFL gene, wherein the endogenous TFL gene: (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, optionally, wherein the mutated endogenous TFL gene produces a truncated TFL polypeptide or no detectable TFL polypeptide.

Further provided is a Rosaceae plant or part thereof comprising a nucleic acid comprising a mutated endogenous TFL gene as described herein.

Also provided are Rosaceae plants comprising in their genome one or more mutated TFL genes and exhibiting a reduced time to flowering, a longer duration of flowering, one or more improved yield characteristics and/or a more determinate plant growth pattern, which Rosaceae plants are produced by the methods described herein, as well as polypeptides, polynucleotides, nucleic acid constructs, expression cassettes and vectors for making a Rosaceae plant of this invention.

These and other aspects of the invention are set forth in more detail in the description of the invention below.

### BRIEF DESCRIPTION OF THE SEQUENCES

SEQ ID NOs:1-17 are exemplary Cas12a amino acid sequences useful with this invention.

SEQ ID NOs:18-20 are exemplary Cas12a nucleotide sequences useful with this invention.

SEQ ID NO:21-22 are exemplary regulatory sequences encoding a promoter and intron.

SEQ ID NOs:23-29 are exemplary cytosine deaminase sequences useful with this invention.

SEQ ID NOs:30-40 are exemplary adenine deaminase amino acid sequences useful with this invention.

SEQ ID NO:41 is an exemplary uracil-DNA glycosylase inhibitor (UGI) sequences useful with this invention.

SEQ ID NOs:42-44 provides an example of a protospacer adjacent motif position for a Type V CRISPR-Cas12a nuclease.

SEQ ID NOs:45-47 provide example peptide tags and affinity polypeptides useful with this invention.

SEQ ID NOs:48-58 provide example RNA recruiting motifs and corresponding affinity polypeptides useful with this invention.

SEQ ID NOs:59-60 are exemplary Cas9 polypeptide sequences useful with this invention.

SEQ ID NOs:61-71 are exemplary Cas9 polynucleotide sequences useful with this invention.

SEQ ID NOs:72, 106, 114, 126, 140, 162, 172, or 184 are example TFL genomic sequences useful with this invention.

SEQ ID NOs: 73, 107, 115, 127, 141, 153, 163, 173, or 185 are example TFL coding sequences (cds) useful with this invention.

SEQ ID NOs: 74, 108, 116, 128, 142, 154, 164, 174, or 186, or are example TFL polypeptide sequences useful with this invention.

SEQ ID NOs:75-105, 109-113, 117-125, 129-139, 143-152, 155-161, 165-171, 175-183, or 187-192 provide example portions or regions of TFL genomic sequences useful with this invention.

SEQ ID NOs:193-197 provide example portions or regions of a TFL polypeptide.

SEQ ID NOs:198-210 are example spacer sequences for CRISPR-Cas guides (Cas12) useful with this invention.

SEQ ID NOs:211-213 are example spacer sequences for CRISPR-Cas guides (Cas9) useful with this invention.

SEQ ID NO:214 and SEQ ID NO:215 are a TFL consensus genomic sequence and a TFL consensus coding sequence, respectively, from the blackberry line A.

SEQ ID NO:216 is a TFL consensus coding TFL polypeptide from the blackberry line A.

SEQ ID NOs:217-233 are example portions or regions of TFL genomic sequences from the blackberry line A useful with this invention.

SEQ ID NOs:247, 271-274 and 275 provide example portions or regions of a TFL polypeptide from the blackberry line A.

SEQ ID NO:234 and SEQ ID NO:235 are a TFL consensus genomic sequence and a TFL consensus coding sequence, respectively, from the blackberry line B.

SEQ ID NO:236 is a TFL consensus coding TFL polypeptide from the blackberry line B.

SEQ ID NOs:237-246 are example portions or regions of TFL genomic sequences from the blackberry line B useful with this invention.

SEQ ID NOs: 248, 271-274, and 276 provide example portions or regions of a TFL polypeptide from the blackberry line B.

SEQ ID NO:252 is a TFL consensus genomic sequence from the blackberry line C.

SEQ ID NO:253 is a TFL consensus coding TFL polypeptide from the blackberry line C.

SEQ ID NOs:254-270 are example portions or regions of TFL genomic sequences from the blackberry line C useful with this invention.

SEQ ID NOs: 247, 271-274, and 275 provide example portions or regions of a TFL polypeptide from the blackberry line C.

SEQ ID NOs:249-251 are example spacer sequences for CRISPR-Cas guides useful with this invention.

SEQ ID NOs:277-284, 295, 297, 298, 300, 302, 304, 306, 308, 309, 310, 311, and 313 are example mutated TFL1 genes.

SEQ ID NOs:285-291, 296, 299, 301, 303, 305, 307, and 312 are example mutated TFL1 polypeptides encoded by SEQ ID NOs:277-284, 295, 297, 298, 300, 302, 304, 306, 308, 309, 310, 311, and 313.

SEQ ID NOs:292-294 are example portions deleted from TFL1 genes.

### BRIEF DESCRIPTION OF THE DRAWINGS

FIG. 1 provides an alignment between the amino acid sequences of SEQ ID NOs:74, 108, 116, 128, 142, 154, 164, and 174.

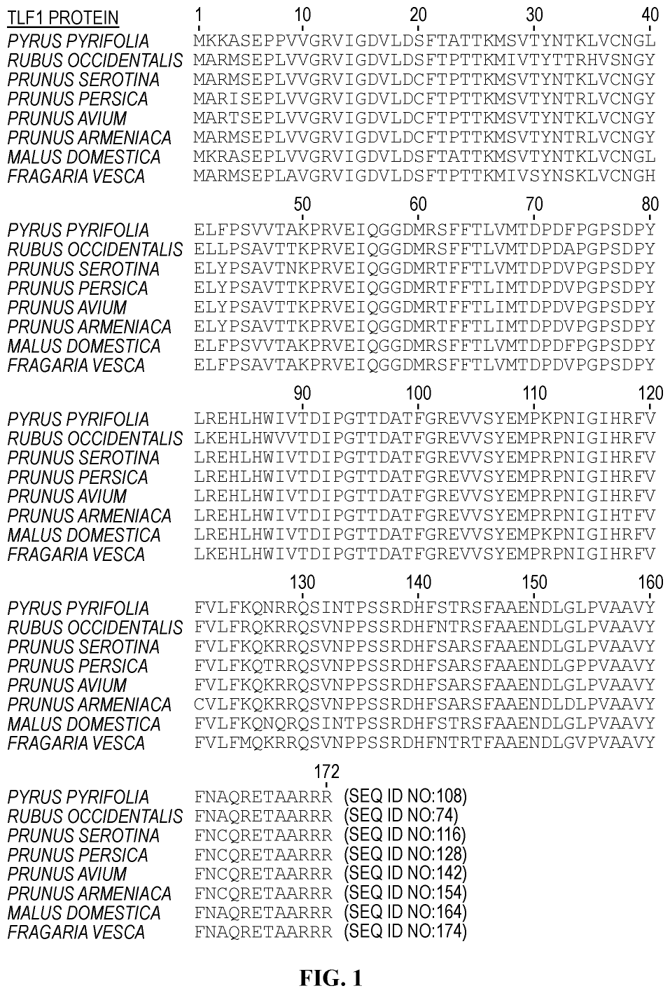

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, +5%, +1%, +0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In particular embodiments, the reduction can result in no or essentially no (i.e., an insignificant amount, e.g., less than about 10% or even 5%) detectable activity or amount. For example, “reduced time to flowering,” or “reduced time to initiation of flowering” means a reduction in the time to flower initiation (e.g., in a Rosaceae plant) of about 5% to about 95%, (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95%; e.g., about 5% to about 75%, about 5% to about 80%, about 5% to about 85%, about 5% to about 90%, about 5% to about 95%, about 15% to about 80%, about 15% to about 85%, about 25% to about 75%, about 25% to about 80%, about 25% to about 85%, about 25% to about 90%, about 25% to about 95%, about 50% to about 80%, about 50% to about 85%, about 75% to about 80%, about 75% to about 85%, about 75% to about 90%, about 75% to about 95%, about 80% to about 85%, about 80% to about 95%), or any range or value therein, as compared to a control plant not comprising the same mutation. A control plant is typically the same plant as the edited plant, but the control plant has not been similarly edited and therefore is devoid of the mutation. A control plant maybe an isogenic plant and/or a wild type plant. Thus, a control plant can be the same breeding line, variety, or cultivar as the subject plant into which a mutation as described herein is introgressed, but the control breeding line, variety, or cultivar is free of the mutation. In some embodiments, a comparison between a plant of the invention and a control plant is made under the same growth conditions, e.g., the same environmental conditions (soil, hydration, light, heat, nutrients, and the like).

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

A “heterologous” or a “recombinant” nucleotide sequence is a nucleotide sequence not naturally associated with a host cell into which it is introduced, including non-naturally occurring multiple copies of a naturally occurring nucleotide sequence. A “heterologous” nucleotide/polypeptide may originate from a foreign species, or, if from the same species, is substantially modified from its native form in composition and/or genomic locus by deliberate human intervention.

A “native” or “wild type” nucleic acid, nucleotide sequence, polypeptide or amino acid sequence refers to a naturally occurring or endogenous nucleic acid, nucleotide sequence, polypeptide or amino acid sequence. In some contexts, a “wild type” nucleic acid is a nucleic acid that is not edited as described herein and can differ from an “endogenous” gene that may be edited as described herein (e.g., a mutated endogenous gene). In some contexts, a “wild type” nucleic acid (e.g., unedited) may be heterologous to the organism in which the wild type nucleic acid is found (e.g., a transgenic organism). As an example, a “wild type endogenous TFL gene” is a TFL gene that is naturally occurring in or endogenous to the reference organism, e.g., a plant in the Rosaceae family, and may be subject to modification as described herein, after which, such a modified endogenous gene is no longer wild type.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus.

A “null allele” is a nonfunctional allele caused by a genetic mutation that results in a complete lack of production of the corresponding protein or produces a protein that is non-functional.

A “recessive mutation” is a mutation in a gene that produces a phenotype when homozygous but the phenotype is not observable when the locus is heterozygous.

A “dominant mutation” is a mutation in a gene that produces a mutant phenotype in the presence of a non-mutated copy of the gene. A dominant mutation may be a loss or a gain of function mutation, a hypomorphic mutation, a hypermorphic mutation or a weak loss of function or a weak gain of function.

A “dominant negative mutation” is a mutation that produces an altered gene product (e.g., having an aberrant function relative to wild type), which gene product adversely affects the function of the wild-type allele or gene product. For example, a “dominant negative mutation” may block a function of the wild type gene product. A dominant negative mutation may also be referred to as an “antimorphic mutation.”

A “semi-dominant mutation” refers to a mutation in which the penetrance of the phenotype in a heterozygous organism is less than that observed for a homozygous organism.

A “weak loss-of-function mutation” is a mutation that results in a gene product having partial function or reduced function (partially inactivated) as compared to the wildtype gene product.

A “hypomorphic mutation” is a mutation that results in a partial loss of gene function, which may occur through reduced expression (e.g., reduced protein and/or reduced RNA) or reduced functional performance (e.g., reduced activity), but not a complete loss of function/activity. A “hypomorphic” allele is a semi-functional allele caused by a genetic mutation that results in production of the corresponding protein that functions at anywhere between 1% and 99% of normal efficiency.

A “hypermorphic mutation” is a mutation that results in increased expression of the gene product and/or increased activity of the gene product.

As used herein, a “non-natural mutation” refers to a mutation that is generated though human intervention and differs from mutations found in the same gene that have occurred in nature (e.g., occurred naturally).

The terms “determinate” or “indeterminate” are used herein in reference to the growth habit of a plant shoot. An “indeterminate” shoot meristem refers to a shoot meristem that continues to grow (no defined end status). A “determinate” shoot meristem refers to a shoot meristem that grows to a fixed length or for fixed length of time. The term “determinate plant growth” refers to plant growth in which the main stem ends in an inflorescence or other reproductive structure (e.g., a bud) and stops continuing to elongate indefinitely with only branches from the main stem having further and similarly restricted growth, e.g., growth characterized by sequential flowering from the central or uppermost bud to the lateral or basal buds. The term “indeterminate plant growth” refers to plant growth in which the main stem continues to elongate indefinitely without being limited by a terminal inflorescence or other reproductive structure, e.g., growth characterized by sequential flowering from the lateral or basal buds to the central or uppermost buds, e.g., the growth of the axis of the plant is not limited by a reproductive structure.

A “more determinate plant growth pattern” refers to a plant that has more shoot meristems that grows to a fixed length or for fixed length of time than a control plant devoid of the mutation in the endogenous TFL gene. In some embodiments, the number of shoot meristems that grow to a fixed length or for a fixed length of time are increased by 50% to 100% (about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%) as compared to a control plant devoid of the mutation in the endogenous TFL gene. In some embodiments, a plant of this invention having a mutation as described herein and a more determinate growth pattern may have stems (about 50% to about 100% of stems) that are about 30% to about 85% or more shorter than a control plant. Thus, a plant of this invention having a mutation as described herein and a more determinate growth pattern can have a more bushy growth habit and can be about 30% to about 85% shorter than a control plant (e.g., having stems about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85% shorter).

A “locus” is a position on a chromosome where a gene or marker or allele is located. In some embodiments, a locus may encompass one or more nucleotides.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with increased yield under non-water stress conditions may be introgressed from a donor into a recurrent parent that does not comprise the marker and does not exhibit increased yield under non-water stress conditions. The resulting offspring could then be backcrossed one or more times and selected until the progeny possess the genetic marker(s) associated with increased yield under non-water stress conditions in the recurrent parent background.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.).

As used herein, the terms “cultivar” and “variety” refer to a group of similar plants that by structural or genetic features and/or performance can be distinguished from other varieties within the same species.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterologous” refers to a nucleotide/polypeptide that originates from a foreign species, or, if from the same species, is substantially modified from its native form in composition and/or genomic locus by deliberate human intervention.

The terms “reduced time to flowering,” “reduced time to flower initiation,” or “reduced period of juvenility” (and grammatical variations thereof) will be used interchangeably herein and mean a shorter time to flowering (e.g., to begin or initiate the flowering process or to begin the growth stage of flowering). The length of time before a plant initiates the flowering process can vary between species and even between different varieties and cultivars. For example, in general, a floricane flowering Rubus plant flowers and produces fruit within about 15-20 months (e.g., 15, 15.5, 16, 16.5, 17, 17.5, 18, 18.5, 19, 19.5, or 20 months) of cane emergence and a primocane flowering Rubus plant flowers and produces fruit after about 3-5 months (e.g., about 3, 3.25, 3.5, 3.75, 4, 4.25, 4.5, 4.75, or 5 months and any range or value therein, e.g., about 12 weeks to about 15 weeks and any range or value therein, e.g., about 12, 13, 14, or 15 weeks and any range or value therein) of cane growth. In contrast, Rubus plants of the present invention can provide a reduction in time to flowering from about 15-20 months to about 6-9 months and any range or value therein of cane emergence (e.g., about 6, 6.25, 6.5, 6.75, 7, 7.25, 7.5, 7.75, 8, 8.25, 8.5, 8.75, or 9 months and any range or value therein, e.g., about 24 weeks to about 36 weeks and any range or value therein, e.g., about 24, 25, 26, 27, 28, 29, 30, 31, 32, 33, 34, 35, or 36 weeks and any range or value therein) as compared to a floricane flowering Rubus plant not comprising a mutation in an endogenous TFL gene as described herein. In some embodiments, Rubus plants of the present invention can provide a reduction in time to flowering from about 3-5 months to about 1-2 months of cane growth in a primocane flowering variety (e.g., about 1, 1.25, 1.5, 1.75, or 2 months and any range or value therein, e.g., about 4 weeks to about 8 weeks and any range or value therein, e.g., about 4, 5, 6, 7, or 8 weeks and any range or value therein) as compared to a primocane flowering Rubus plant not comprising a mutation in an endogenous TFL gene as described herein.

Thus, in some embodiments, a reduced time to flowering in a Rosaceae plant may be about 5% to about 95%, (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95% or any range or value therein; e.g., about 5% to about 20% (e.g., about 5, 6, 7, 8, 9, 10, 11, 12, 13, 14, 15, 16, 17, 18, 19, or 20%), about 5% to about 25%, (e.g., about 5, 6, 7, 8, 9, 10, 11, 12, 13, 14, 15, 16, 17, 18, 19, 20, 21, 22, 23, 24, or 25%), about 5% to about 30% (e.g., about 5,6, 7, 8, 9, 10, 11, 12, 13, 14, 15, 16, 17, 18, 19, 20, 21, 22, 23, 24, 25, 26, 27, 28, 29, or 30%), about 5% to about 50%, about 5% to about 75%, about 5% to about 80%, about 5% to about 85%, about 5% to about 90%, about 5% to about 95%, about 15% to about 80%, about 15% to about 85%, about 25% to about 75%, about 25% to about 80%, about 25% to about 85%, about 25% to about 90%, about 25% to about 95%, about 50% to about 80%, about 50% to about 85%, about 75% to about 80%, about 75% to about 85%, about 75% to about 90%, about 75% to about 95%, about 80% to about 85%, about 80% to about 95%), or any range or value therein, as compared to a control plant not comprising the same mutation. In some embodiments, the present invention provides a reduced time to flowering in a Rubus plant of about to 5%-50% (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% or any range or value therein, optionally about 5%-25%) as compared to a wild type Rubus (e.g., a Rubus plant not comprising a mutation in an endogenous TFL gene as described herein). Thus, in some embodiments, a Rubus plant of the present invention will flower and/or produce fruit within 2-9 months (e.g., 2, 2.5, 3, 3.5, 4, 4.5 5, 5.5, 6, 6.5, 7, 7.5, 8, 8.5, or 9 months) of cane emergence. For a Prunus plant (e.g., cherry) comprising a mutation as described herein, time to flowering may be reduced by 40% to about 90% (e.g., about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90%, or any range or value therein), optionally about 60% to about 85% as compared to a wild type Prunus (e.g., a Prunus plant not comprising a mutation in an endogenous TFL gene as described herein). As an example, the methods of present invention may provide blackberry plants that fruit on new wood. That is, a blackberry plant of the invention may flower and produce fruit on canes produced the same year that the cane is produced instead of the second year (e.g., about 18 months or longer after cane emergence)—that is, a blackberry plant of the invention may flower on new wood rather than old wood, thereby reducing the time to flowering in the blackberry plant. Similarly, other Rosaceae plants having a mutation as described herein may also exhibit a reduced time to flowering. In some embodiments, a Rosaceae plant of the invention may further exhibit a phenotype of a longer duration of flowering, one or more (e.g., 1, 2, 3, 4, 5, 6, or more) improved yield characteristics (e.g., increased fruit production) and/or a more determinate plant growth pattern.

The term “longer duration of flowering” refers to the period from the initiation of flowering to the end of flowering (e.g., last flower produced) is longer as compared to a Rosaceae plant (e.g., Rubus plant, Prunus plant, Fragaria plant, a Malus plant, etc.) that does not comprise at least one mutation in an endogenous TFL gene as described herein. For example, a Rosaceae plant that comprises one or more mutations in one or more endogenous TFL genes as described herein may have a duration of flowering that is at least one day to at least 5 weeks (e.g., 1, 2, 3, 4, 5, 6, 7 days to about 1, 2, 3, 4, or 5 weeks; e.g., about 1, 2, 3, 4, 5, 6, 7, 8, 9, 10, 11, 12, 13, 14, 15 days to about 16, 17, 18, 19, 20, 21, 22, 23, 24, 25, 26, 27, 28, 29, 30 31, 32, 33, 34, 35 days; e.g., about 1, 2, 3, 4, or 5 weeks) longer than a Rosaceae plant that does not comprise one or more TFL mutations. For example, a blackberry plant typically flowers over a period of 3 weeks (e.g., about 21 days). Thus, in some embodiments, a blackberry plant of this invention will flower for longer than 3 weeks by one or more days as compared to blackberry plant that does not comprise at least one mutation in an endogenous TFL gene as described herein. In some embodiments, a blackberry plant of this invention will flower for longer than 3 weeks by at least 5 days to about 5 weeks as compared to blackberry plant that does not comprise at least one mutation in an endogenous TFL gene as described herein.

As used herein a “control plant” means a plant that does not contain an edited TFL gene or genes as described herein that imparts an enhanced/improved trait (e.g., yield trait) or altered phenotype. A control plant is used to identify and select a plant edited as described herein and that has an enhanced trait or altered phenotype as compared to the control plant. A suitable control plant can be a plant of the parental line used to generate a plant comprising a mutated TFL gene(s), for example, a Rosaceae plant (e.g., Rubus plant, Prunus plant, Fragaria plant, a Malus plant, etc.) devoid of an edit in an endogenous TFL gene as described herein. A suitable control plant can also be a plant that contains recombinant nucleic acids that impart other traits, for example, a transgenic plant having enhanced herbicide tolerance. A suitable control plant can in some cases be a progeny of a heterozygous or hemizygous transgenic plant line that is devoid of the mutated TFL gene as described herein, known as a negative segregant, or a negative isogenic line.

An enhanced trait (e.g., improved yield trait) may include, for example, decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, increased yield, increased nitrogen use efficiency, and increased water use efficiency as compared to a control plant. An altered phenotype may be, for example, plant height, biomass, canopy area, anthocyanin content, chlorophyll content, water applied, water content, and water use efficiency.

In some embodiments, a plant of this invention may comprise one or more improved yield traits including, but not limited to, higher yield (bu/acre), increased biomass, increased plant height, increased stem diameter, increased leaf area, increased number of flowers, increased number of pods, including an increased number of pods per node and/or an increased number of pods per plant, increased number of seeds per pod, increase number of seeds, increased seed size, and/or increased seed weight (e.g., increase in 100-seed weight) as compared to a control plant devoid of the at least one mutation. In some embodiments, an improved yield trait or characteristic may be, for example, increased fruit production on a per plant or per hectare basis.

As used herein a “trait” is a physiological, morphological, biochemical, or physical characteristic of a plant or particular plant material or cell. In some instances, this characteristic is visible to the human eye and can be measured mechanically, such as seed or plant size, weight, shape, form, length, height, growth rate and development stage, or can be measured by biochemical techniques, such as detecting the protein, starch, certain metabolites, or oil content of seed or leaves, or by observation of a metabolic or physiological process, for example, by measuring tolerance to water deprivation or particular salt or sugar concentrations, or by the measurement of the expression level of a gene or genes, for example, by employing Northern analysis, RT-PCR, microarray gene expression assays, or reporter gene expression systems, or by agricultural observations such as hyperosmotic stress tolerance or yield. However, any technique can be used to measure the amount of, the comparative level of, or the difference in any selected chemical compound or macromolecule in the transgenic plants.

As used herein an “enhanced trait” means a characteristic of a plant resulting from mutations in a TFL gene(s) as described herein. Such traits include, but are not limited to, an enhanced agronomic trait characterized by enhanced plant morphology, physiology, growth and development, yield, nutritional enhancement, disease or pest resistance, or environmental or chemical tolerance. In some embodiments, an enhanced trait/altered phenotype may be, for example, decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, drought tolerance, increased water use efficiency, cold tolerance, increased nitrogen use efficiency, and increased yield. In some embodiments, a trait is increased yield under nonstress conditions or increased yield under environmental stress conditions. Stress conditions can include both biotic and abiotic stress, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, plant biomass, pod number, pod position on the plant, number of internodes, incidence of pod shatter, grain size, ear size, ear tip filling, kernel abortion, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), flowering time and duration, ear number, ear size, ear weight, seed number per ear or pod, seed size, composition of seed (starch, oil, protein) and characteristics of seed fill.

Also used herein, the term “trait modification” encompasses altering the naturally occurring trait by producing a detectable difference in a characteristic in a plant comprising a mutation in an endogenous TFL gene as described herein relative to a plant not comprising the mutation, such as a wild-type plant, or a negative segregant. In some cases, the trait modification can be evaluated quantitatively. For example, the trait modification can entail an increase or decrease in an observed trait characteristics or phenotype as compared to a control plant. It is known that there can be natural variations in a modified trait. Therefore, the trait modification observed entails a change of the normal distribution and magnitude of the trait characteristics or phenotype in the plants as compared to a control plant.

The present disclosure relates to a plant with improved economically relevant characteristics, more specifically increased yield. More specifically the present disclosure relates to a plant comprising a mutation(s) in a TFL gene(s) as described herein, wherein the plant has increased yield as compared to a control plant devoid of said mutation(s). In some embodiments, plants produced as described herein exhibit increased yield or improved yield trait components as compared to a control plant. In some embodiments, a plant of the present disclosure exhibits an improved trait that is related to yield, including but not limited to increased nitrogen use efficiency, increased nitrogen stress tolerance, increased water use efficiency and increased drought tolerance, as defined and discussed infra.

Yield can be defined as the measurable produce of economic value from a crop. Yield can be defined in the scope of quantity and/or quality. Yield can be directly dependent on several factors, for example, the number and size of organs, plant architecture (such as the number of branches, plant biomass, e.g., increased root biomass, steeper root angle and/or longer roots, and the like), flowering time and duration, grain fill period. Root architecture and development, photosynthetic efficiency, nutrient uptake, stress tolerance, early vigor, delayed senescence and functional stay green phenotypes may be factors in determining yield. Optimizing the above-mentioned factors can therefore contribute to increasing crop yield.

Reference herein to an increase/improvement in yield-related traits can also be taken to mean an increase in biomass (weight) of one or more parts of a plant, which can include above ground and/or below ground (harvestable) plant parts. In particular, such harvestable parts are seeds, and performance of the methods of the disclosure results in plants with increased yield and in particular increased seed yield relative to the seed yield of suitable control plants. The term “yield” of a plant can relate to vegetative biomass (root and/or shoot biomass), to reproductive organs, and/or to propagules (such as seeds) of that plant.

Increased yield of a plant of the present disclosure can be measured in a number of ways, including test weight, seed number per plant, seed weight, seed number per unit area (for example, seeds, or weight of seeds, per acre), bushels per acre, tons per acre, or kilo per hectare. Increased yield can result from improved utilization of key biochemical compounds, such as nitrogen, phosphorous and carbohydrate, or from improved responses to environmental stresses, such as cold, heat, drought, salt, shade, high plant density, and attack by pests or pathogens.

“Increased yield” can manifest as one or more of the following: (i) increased plant biomass (weight) of one or more parts of a plant, particularly aboveground (harvestable) parts, of a plant, increased root biomass (increased number of roots, increased root thickness, increased root length) or increased biomass of any other harvestable part; or (ii) increased early vigor, defined herein as an improved seedling aboveground area approximately three weeks post-germination.

“Early vigor” refers to active healthy plant growth especially during early stages of plant growth, and can result from increased plant fitness due to, for example, the plants being better adapted to their environment (for example, optimizing the use of energy resources, uptake of nutrients and partitioning carbon allocation between shoot and root). Early vigor, for example, can be a combination of the ability of seeds to germinate and emerge after planting and the ability of the young plants to grow and develop after emergence. Plants having early vigor also show increased seedling survival and better establishment of the crop, which often results in highly uniform fields with the majority of the plants reaching the various stages of development at substantially the same time, which often results in increased yield. Therefore, early vigor can be determined by measuring various factors, such as kernel weight, percentage germination, percentage emergence, seedling growth, seedling height, root length, root and shoot biomass, canopy size and color and others.

Further, increased yield can also manifest as increased total seed yield, which may result from one or more of an increase in seed biomass (seed weight) due to an increase in the seed weight on a per plant and/or on an individual seed basis an increased number of, for example, flowers/panicles per plant; an increased number of pods; an increased number of nodes; an increased number of flowers (“florets”) per panicle/plant; increased seed fill rate; an increased number of filled seeds; increased seed size (length, width, area, perimeter, and/or weight), which can also influence the composition of seeds; and/or increased seed volume, which can also influence the composition of seeds. In one embodiment, increased yield can be increased seed yield, for example, increased seed weight; increased number of filled seeds; and increased harvest index.

Increased yield can also result in modified architecture, or can occur because of modified plant architecture.

Increased yield can also manifest as increased harvest index, which is expressed as a ratio of the yield of harvestable parts, such as seeds, over the total biomass

The disclosure also extends to harvestable parts of a plant such as, but not limited to, seeds, leaves, fruits, flowers, bolls, pods, siliques, nuts, stems, rhizomes, tubers and bulbs. The disclosure furthermore relates to products derived from a harvestable part of such a plant, such as dry pellets, powders, oil, fat and fatty acids, starch or proteins.

The present disclosure provides a method for increasing “yield” of a plant or “broad acre yield” of a plant or plant part defined as the harvestable plant parts per unit area, for example seeds, or weight of seeds, per acre, pounds per acre, bushels per acre, tones per acre, tons per acre, kilo per hectare.

As used herein “nitrogen use efficiency” refers to the processes which lead to an increase in the plant's yield, biomass, vigor, and growth rate per nitrogen unit applied. The processes can include the uptake, assimilation, accumulation, signaling, sensing, retranslocation (within the plant) and use of nitrogen by the plant.

As used herein “increased nitrogen use efficiency” refers to the ability of plants to grow, develop, or yield faster or better than normal when subjected to the same amount of available/applied nitrogen as under normal or standard conditions; ability of plants to grow, develop, or yield normally, or grow, develop, or yield faster or better when subjected to less than optimal amounts of available/applied nitrogen, or under nitrogen limiting conditions.

As used herein “nitrogen limiting conditions” refers to growth conditions or environments that provide less than optimal amounts of nitrogen needed for adequate or successful plant metabolism, growth, reproductive success and/or viability.

As used herein the “increased nitrogen stress tolerance” refers to the ability of plants to grow, develop, or yield normally, or grow, develop, or yield faster or better when subjected to less than optimal amounts of available/applied nitrogen, or under nitrogen limiting conditions.

Increased plant nitrogen use efficiency can be translated in the field into either harvesting similar quantities of yield, while supplying less nitrogen, or increased yield gained by supplying optimal/sufficient amounts of nitrogen. The increased nitrogen use efficiency can improve plant nitrogen stress tolerance and can also improve crop quality and biochemical constituents of the seed such as protein yield and oil yield. The terms “increased nitrogen use efficiency”, “enhanced nitrogen use efficiency”, and “nitrogen stress tolerance” are used inter-changeably in the present disclosure to refer to plants with improved productivity under nitrogen limiting conditions.

As used herein “water use efficiency” refers to the amount of carbon dioxide assimilated by leaves per unit of water vapor transpired. It constitutes one of the most important traits controlling plant productivity in dry environments. “Drought tolerance” refers to the degree to which a plant is adapted to arid or drought conditions. The physiological responses of plants to a deficit of water include leaf wilting, a reduction in leaf area, leaf abscission, and the stimulation of root growth by directing nutrients to the underground parts of the plants. Typically, plants are more susceptible to drought during flowering and seed development (the reproductive stages), as plant's resources are deviated to support root growth. In addition, abscisic acid (ABA), a plant stress hormone, induces the closure of leaf stomata (microscopic pores involved in gas exchange), thereby reducing water loss through transpiration, and decreasing the rate of photosynthesis. These responses improve the water-use efficiency of the plant on the short term. The terms “increased water use efficiency”, “enhanced water use efficiency”, and “increased drought tolerance” are used inter-changeably in the present disclosure to refer to plants with improved productivity under water-limiting conditions.

As used herein “increased water use efficiency” refers to the ability of plants to grow, develop, or yield faster or better than normal when subjected to the same amount of available/applied water as under normal or standard conditions; ability of plants to grow, develop, or yield normally, or grow, develop, or yield faster or better when subjected to reduced amounts of available/applied water (water input) or under conditions of water stress or water deficit stress.

As used herein “increased drought tolerance” refers to the ability of plants to grow, develop, or yield normally, or grow, develop, or yield faster or better than normal when subjected to reduced amounts of available/applied water and/or under conditions of acute or chronic drought; ability of plants to grow, develop, or yield normally when subjected to reduced amounts of available/applied water (water input) or under conditions of water deficit stress or under conditions of acute or chronic drought.

As used herein, “drought stress” refers to a period of dryness (acute or chronic/prolonged) that results in water deficit and subjects plants to stress and/or damage to plant tissues and/or negatively affects grain/crop yield; a period of dryness (acute or chronic/prolonged) that results in water deficit and/or higher temperatures and subjects plants to stress and/or damage to plant tissues and/or negatively affects grain/crop yield.

As used herein, “water deficit” refers to the conditions or environments that provide less than optimal amounts of water needed for adequate/successful growth and development of plants.

As used herein, “water stress” refers to the conditions or environments that provide improper (either less/insufficient or more/excessive) amounts of water than that needed for adequate/successful growth and development of plants/crops thereby subjecting the plants to stress and/or damage to plant tissues and/or negatively affecting grain/crop yield.

As used herein “water deficit stress” refers to the conditions or environments that provide less/insufficient amounts of water than that needed for adequate/successful growth and development of plants/crops thereby subjecting the plants to stress and/or damage to plant tissues and/or negatively affecting grain yield.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length relative (e.g., reduced by 1, 2, 3, 4, 5, 6, 7, 8, 9, 10, 11, 12, 13, 14, 15, 16, 17, 18, 19, 20, 20, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, or 900 or more nucleotides or any range or value therein) to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. As an example, a repeat sequence of guide nucleic acid of this invention may comprise a “portion” of a wild type CRISPR-Cas repeat sequence (e.g., a wild Type CRISR-Cas repeat; e.g., a repeat from the CRISPR Cas system of, for example, a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like).

In some embodiments, a nucleic acid fragment may comprise, consist essentially of or consist of about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, 550, 600, 650, 700, 750, 800, 850, or 900 or more consecutive nucleotides or any range or value therein of a nucleic acid encoding a TFL protein, optionally a TFL fragment may be about 50 nucleotides to about 300 nucleotides in length, about 50 nucleotides to about 350 nucleotides in length, about 50 nucleotides to about 400 nucleotides in length, about 50 nucleotides to about 450 nucleotides in length, about 50 nucleotides to about 500 nucleotides in length, about 50 nucleotides to about 600 nucleotides in length, about 50 nucleotides to about 800 nucleotides in length, about 50 nucleotides to about 900 nucleotides in length, about 50 nucleotides to about 950 nucleotides in length, about 100 nucleotides to about 300 nucleotides in length, about 100 nucleotides to about 350 nucleotides in length, about 100 nucleotides to about 400 nucleotides in length, about 100 nucleotides to about 450 nucleotides in length, about 100 nucleotides to about 500 nucleotides in length, about 100 nucleotides to about 600 nucleotides in length, about 100 nucleotides to about 800 nucleotides in length, about 100 nucleotides to about 900 nucleotides in length, or about 100 nucleotides to about 950 nucleotides in length, or any range or value therein. (e.g., a fragment or portion of any one of SEQ ID NOs:72, 73, 106, 107, 114, 115, 126, 127, 140, 141, 153, 162, 163, 172, 173, 184, 185, 214, 215, 234, 235, or 252 (e.g., SEQ ID NOs:75-105, 109-113, 117-125, 129-139, 143-152, 155-161, 165-171, 175-183, 187-192, 217-233, 237-246, or 254-270).

In some embodiments, a “portion” in reference to a nucleic acid means at least 2, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 135, 136, 137, 138, 139, 140, 141, 142, 143, 144, 145, 150, 160, 170, 171, 172, 173, 174, 175, 176, 177, 178, 179, 180, 190, 200, 210, 215, 216, 217, 218, 219, 220, 221, 222, 223, 224, 225, 230, 240, 250, 260, 270, 280, 290, 291, 292, 293, 294, 295,296, 297, 298, 299, or 300 or more consecutive nucleotides from a gene (e.g., consecutive nucleotides from a TFL gene) (e.g., a fragment or portion of any one of SEQ ID NOs:72, 73, 106, 107, 114, 115, 126, 127, 140, 141, 153, 162, 163, 172, 173, 184, 185, 214, 215, 234, 235, or 252 (e.g., SEQ ID NOs:75-105, 109-113, 117-125, 129-139, 143-152, 155-161, 165-171, 175-183, 187-192, 217-233, 237-246, or 254-270).

In some embodiments, a nucleic acid fragment of a TFL gene may be the result of a deletion of nucleotides from the 3′ end, the 5′ end, and/or from within any region (e.g., within an exon, a coding region) of a gene encoding a TFL protein. In some embodiments, a deletion of a portion of a gene encoding a TFL protein may comprise a deletion of a portion of consecutive nucleotides from the 5′ end, the 3′ end, or from within any region of a gene, for example, a deletion of a portion of consecutive nucleotides from the 5′ end, the 3′ end, or from within any region of any one of the nucleotide sequences of SEQ ID NOs:72, 73, 106, 107, 114, 115, 126, 127, 140, 141, 153, 162, 163, 172, 173, 184, 185, 214, 215, 234, 235, or 252. In some embodiments, a deletion of a portion of a TFL gene may comprise deletion of a portion of consecutive nucleotides from any one of the nucleotide sequences of SEQ ID NOs:72, 73, 106, 107, 114, 115, 126, 127, 140, 141, 153, 162, 163, 172, 173, 184, 185, 214, 215, 234, 235, or 252.

In some embodiments, a deletion of a portion of a TFL gene may comprise a deletion of a portion of consecutive nucleotides from any one of the nucleotide sequences of SEQ ID NOs:72, 106, 114, 126, 140, 162, 172, 184, 214, 234, or 252 from about 3 consecutive nucleotides to about 2600 consecutive nucleotides or more (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175,200, 225, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500 or 2600 or more consecutive nucleotides, or any range or value therein).

In some embodiments, a deletion of a TFL gene may comprise a deletion of about 3 to about 515 or more nucleotides of SEQ ID NOs:73, 107, 115, 127, 141, 153, 163, 173, or 185, 215, or 235 (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 consecutive nucleotides to about 100, 101, 102, 103, 104, 105, 106, 107, 108, 109, 110, 111, 112, 113, 114, 115, 116, 117, 118, 119, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 350, 455, 460, 465, 470, 475, 480, 485, 490, 495, 500, 505, 510, 515, 516, 517, 518, 519, 520 or more consecutive nucleotides or any range or value therein, optionally about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 to about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 consecutive nucleotides).

In some embodiments, a deletion in a TFL gene as described herein may be a null allele, which when comprised in a plant can result in the plant exhibiting a phenotype of reduced time to flowering in the plant, a longer duration of flowering, one or more improved yield characteristics and/or a more determinate plant growth pattern. In some embodiments, such a deletion may be a dominant-negative allele, semi-dominant allele, weak loss of function allele, a null allele, or a hypomorphic mutation, which when comprised in a plant can result in the plant exhibiting a phenotype of reduced time to flowering in the plant, a longer duration of flowering, one or more improved yield characteristics (e.g., increased fruit production) and/or a more determinate plant growth pattern.

In some embodiments, a “sequence-specific nucleic acid binding domain” may bind to one or more fragments or portions of nucleotide sequences (e.g., DNA, RNA) encoding, for example, TFL polypeptides as described herein.

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400 or more consecutive amino acids of a reference polypeptide. In some embodiments, a polypeptide fragment may comprise, consist essentially of or consist of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, or 172 or more consecutive amino acid residues (or any range or value therein) of a TFL1 polypeptide (e.g., a fragment or a portion of any one of SEQ ID NOs:74, 108, 116, 128, 142, 154, 164, 174, 186, 216, 236 or 253 (e.g., SEQ ID NOs:193-197, 247, 248, or 271-276).

In some embodiments, a “portion” may be related to the number of amino acids that are deleted from a polypeptide. Thus, for example, a deleted “portion” of an TFL polypeptide may comprise at least one amino acid residue (e.g., at least 1, or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, or 172, or more consecutive amino acid residues) deleted from any one of the amino acid sequences SEQ ID NOs:74, 108, 116, 128, 142, 154, 164, 174, 186, 216, 236 or 253 (e.g., SEQ ID NOs:193-197, 247, 248, or 271-276).

In some embodiments, a deletion of a portion of a TFL protein may comprise a deletion of a portion of consecutive amino acid residues from the N- or C-terminus of or within any one of the amino acid sequences of SEQ ID NOs:74, 108, 116, 128, 142, 154, 164, 174, 186, 216, 236 or 253 (e.g., SEQ ID NOs:193-197, 247, 248, or 271-276). Thus, in some embodiments, a fragment or portion of a TFL polypeptide that is deleted may be within a TFL polypeptide from amino acid residue 20, 21, 22, 23, 24, 25, 30, 35, 40, 45, 50, 60, 70, 71, 72, 73, or 74 to amino acid residue 115, 116, 117, 118, 119, or 120 with reference amino acid position numbering of SEQ ID NOs:74, 108, 116, 128, 142, 154, 164, or 174, or from amino acid residue 60, 70, 71, 72, 73, 74, 75, 76, 78, 79, 80 or to amino acid residue 117, 118, 119, 120, 121, 122, 123, 124, or 125 with reference amino acid position numbering of SEQ ID NO:186, 216, 236 or 253. In some embodiments, a fragment or portion of a TFL polypeptide that is deleted may comprise the sequence of any one of the amino acid sequences of SEQ ID NOs:193-197, 247, 248, or 271-276, or portion thereof.

In some embodiments, a deletion of a portion of a TFL polypeptide may comprise a deletion of a portion of consecutive amino acid residues from the C-terminus or N-terminus of any one of the amino acid sequences of SEQ ID NOs:74, 108, 116, 128, 142, 154, 164, 174, 186, 216, 236 or 253. In some embodiments, a deletion of a portion of a TFL polypeptide may comprise a deletion of a portion of consecutive amino acid residues from the C-terminus or N-terminus of any one of the amino acid sequences SEQ ID NOs:74, 108, 116, 128, 142, 154, 164, 174, 186, 216, 236 or 253 of from about 1, 2, 3, 4, 5, 6, 7, 8, 9, or 10 consecutive amino acids to about 50, about 75, about 100, about 120, about 130, about 150, about 160, or about 172 consecutive amino acids, or about 5, 6, 7, 8, 9, 10, 11, 12, 13, 14, 15, 16, 17, 18, 19, 20, 21, 22, 23, 24, 25, 26, 27, 28, 29, or 30 consecutive amino acids to about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, or 170 or more consecutive amino acids (or any range or value therein). In some embodiments, such a deletion may be a null allele, which when comprised in a Rosaceae plant (e.g., Rubus, Prunus plant, Fragaria plant, a Malus plant, etc.), may result in a phenotype of reduced time to flowering, a longer duration of flowering, a more determinate plant growth pattern and/or one or more improved yield characteristics (e.g., increased fruit production) compared to a Rosaceae plant devoid of the deletion. In some embodiments, such a deletion may be a dominant-negative allele, semi-dominant allele, weak loss of function allele, a null allele, or a hypomorphic mutation, which when comprised in a Rosaceae (e.g., Rubus, Prunus, Fragaria, Malus, etc.) plant may result in the plant having a reduced time to flowering, a longer duration of flowering, a more determinate plant growth pattern and/or one or more improved yield characteristics compared to a control Rosaceae (e.g., Rubus, Prunus, Fragaria, Malus, etc.) plant devoid of the mutation.

As used herein with respect to nucleic acids, the term “functional fragment” refers to nucleic acid that encodes a functional fragment of a polypeptide. A “functional fragment” with respect to a polypeptide is a fragment of a polypeptide that retains one or more of the activities of the native reference polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, inversions and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue. A truncation can include a truncation at the C-terminal end of a polypeptide or at the N-terminal end of a polypeptide. A truncation of a polypeptide can be the result of a deletion of the corresponding 5′ end or 3′ end of the gene encoding the polypeptide. A frameshift mutation can occur when deletions or insertions of one or more base pairs are introduced into a gene, optionally resulting in an out-of-frame mutation or an in-frame mutation. Frameshift mutations in a gene can result in the production of a polypeptide that is longer, shorter or the same length as the wild type polypeptide depending on when the first stop codon occurs following the mutated region of the gene. As an example, an out-of-frame mutation that produces a premature stop codon can produce a polypeptide that is shorter that the wild type polypeptide, or, in some embodiments, the polypeptide may be absent/undetectable. In some embodiments, a mutation may be a DNA inversion, optionally a DNA inversion having a length of about 10 to about 2000 consecutive base pairs.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences, or polypeptide sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more in length, or any range therein, up to the full length of the sequence. In some embodiments, nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40, 50, 60, 70 or 80 nucleotides or more).

In some embodiments of the invention, the substantial identity exists over a region of consecutive amino acid residues of a polypeptide of the invention that is about 3 amino acid residues to about 20 amino acid residues, about 5 amino acid residues to about 25 amino acid residues, about 7 amino acid residues to about 30 amino acid residues, about 10 amino acid residues to about 25 amino acid residues, about 15 amino acid residues to about 30 amino acid residues, about 20 amino acid residues to about 40 amino acid residues, about 25 amino acid residues to about 40 amino acid residues, about 25 amino acid residues to about 50 amino acid residues, about 30 amino acid residues to about 50 amino acid residues, about 40 amino acid residues to about 50 amino acid residues, about 40 amino acid residues to about 70 amino acid residues, about 50 amino acid residues to about 70 amino acid residues, about 60 amino acid residues to about 80 amino acid residues, about 70 amino acid residues to about 80 amino acid residues, about 90 amino acid residues to about 100 amino acid residues, or more amino acid residues in length, and any range therein, up to the full length of the sequence. In some embodiments, polypeptide sequences can be substantially identical to one another over at least about 8 consecutive amino acid residues (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350 or more amino acids in length or more consecutive amino acid residues). In some embodiments, two or more TFL polypeptides may be identical or substantially identical (e.g., at least 70% to 99.9% identical, e.g., about 70%, 71%, 72%, 73%, 74%, 75%, 76%, 77%, 78%, 79%, 80%, 81%, 82%, 83%, 84%, 85%, 86%, 87%, 88%, 89%0, 90%, 91%, 92%, 93%, 94%, 95%, 96%, 97%, 98%, 99%, 99.5%. 99.9% identical or any range or value therein) to one another over at least about 44 consecutive amino acid residues (e.g., SEQ ID NOs:193-197, 247, 248, or 271-276). In some embodiments, two or more TFL proteins may be substantially identical across consecutive amino acid residues 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 to about 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, 150, 155, 160, 165, 170, 171, 172 or more of any one of the amino acid sequences of SEQ ID NOs:74, 108, 116, 128, 142, 154, 164, 174, 186, 216, 236 or 253.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention (e.g., expression cassettes and/or vectors) may be codon optimized for expression. In some embodiments, the polynucleotides, nucleic acid constructs, expression cassettes, and/or vectors of the editing systems of the invention (e.g., comprising/encoding a sequence-specific DNA binding domain (e.g., a sequence-specific DNA binding domain from a polynucleotide-guided endonuclease, a zinc finger nuclease, a transcription activator-like effector nuclease (TALEN), an Argonaute protein, and/or a CRISPR-Cas endonuclease (e.g., CRISPR-Cas effector protein) (e.g., a Type I CRISPR-Cas effector protein, a Type II CRISPR-Cas effector protein, a Type III CRISPR-Cas effector protein, a Type IV CRISPR-Cas effector protein, a Type V CRISPR-Cas effector protein or a Type VI CRISPR-Cas effector protein)), a nuclease (e.g., an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN)), deaminase proteins/domains (e.g., adenine deaminase, cytosine deaminase), a polynucleotide encoding a reverse transcriptase protein or domain, a polynucleotide encoding a 5′-3′ exonuclease polypeptide, and/or affinity polypeptides, peptide tags, etc.) may be codon optimized for expression in a plant. In some embodiments, the codon optimized nucleic acids, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acids, polynucleotides, expression cassettes, and/or vectors that have not been codon optimized.

A polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a DNA binding polypeptide or domain and peptide tag and/or a reverse transcriptase and an affinity polypeptide that binds to the peptide tag; or a DNA endonuclease polypeptide or domain and peptide tag and/or a reverse transcriptase and an affinity polypeptide that binds to the peptide tag. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker.

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g. extension of the hairpin structure in the guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as 0-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA2-6 promoter from arabidopsis (U.S. Pat. No. 7,141,424), and/or the ZrC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron (see, e.g., SEQ ID NO:21 and SEQ ID NO:22).

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdcal), the psbA gene, the atpA gene, or any combination thereof.

In some embodiments, a polynucleotide and/or a nucleic acid construct of the invention can be an “expression cassette” or can be comprised within an expression cassette.

As used herein, “expression cassette” means a recombinant nucleic acid molecule comprising, for example, a one or more polynucleotides of the invention (e.g., a polynucleotide encoding a sequence-specific DNA binding domain, a polynucleotide encoding a deaminase protein or domain, a polynucleotide encoding a reverse transcriptase protein or domain, a polynucleotide encoding a 5′-3′ exonuclease polypeptide or domain, a guide nucleic acid and/or reverse transcriptase (RT) template), wherein polynucleotide(s) is/are operably associated with one or more control sequences (e.g., a promoter, terminator and the like). Thus, in some embodiments, one or more expression cassettes may be provided, which are designed to express, for example, a nucleic acid construct of the invention (e.g., a polynucleotide encoding a sequence-specific DNA binding domain, a polynucleotide encoding a nuclease polypeptide/domain, a polynucleotide encoding a deaminase protein/domain, a polynucleotide encoding a reverse transcriptase protein/domain, a polynucleotide encoding a 5′-3′ exonuclease polypeptide/domain, a polynucleotide encoding a peptide tag, and/or a polynucleotide encoding an affinity polypeptide, and the like, or comprising a guide nucleic acid, an extended guide nucleic acid, and/or RT template, and the like). When an expression cassette of the present invention comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter, or they may be different promoters. Thus, a polynucleotide encoding a sequence specific DNA binding domain, a polynucleotide encoding a nuclease protein/domain, a polynucleotide encoding a CRISPR-Cas effector protein/domain, a polynucleotide encoding an deaminase protein/domain, a polynucleotide encoding a reverse transcriptase polypeptide/domain (e.g., RNA-dependent DNA polymerase), and/or a polynucleotide encoding a 5′-3′ exonuclease polypeptide/domain, a guide nucleic acid, an extended guide nucleic acid and/or RT template when comprised in a single expression cassette may each be operably linked to a single promoter, or separate promoters in any combination.

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to, for example, a gene encoding a sequence-specific DNA binding protein, a gene encoding a nuclease, a gene encoding a reverse transcriptase, a gene encoding a deaminase, and the like, or may be native to a host cell, or may be native to another source (e.g., foreign or heterologous to, for example, to a promoter, to a gene encoding a sequence-specific DNA binding protein, a gene encoding a nuclease, a gene encoding a reverse transcriptase, a gene encoding a deaminase, and the like, or to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct (e.g., expression cassette(s)) comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g., higher plant, mammalian, yeast, or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As an example, a target nucleic acid may be contacted with a sequence-specific nucleic acid binding protein (e.g., polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein)) and a deaminase or a nucleic acid construct encoding the same, under conditions whereby the sequence-specific DNA binding protein, the reverse transcriptase and/or the deaminase are expressed and the sequence-specific DNA binding protein binds to the target nucleic acid, and the reverse transcriptase and/or deaminase may be fused to either the sequence-specific DNA binding protein or recruited to the sequence-specific DNA binding protein (via, for example, a peptide tag fused to the sequence-specific DNA binding protein and an affinity tag fused to the reverse transcriptase and/or deaminase) and thus, the deaminase and/or reverse transcriptase is positioned in the vicinity of the target nucleic acid, thereby modifying the target nucleic acid. Other methods for recruiting reverse transcriptase and/or deaminase may be used that take advantage of other protein-protein interactions, and also RNA-protein interactions and chemical interactions may be used for protein-protein and protein-nucleic acid recruitment.

As used herein, “modifying” or “modification” in reference to a target nucleic acid includes editing (e.g., mutating), covalent modification, exchanging/substituting nucleic acids/nucleotide bases, deleting, cleaving, nicking, and/or altering transcriptional control of a target nucleic acid. In some embodiments, a modification may include one or more single base changes (SNPs) of any type.

“Introducing,” “introduce,” “introduced” (and grammatical variations thereof) in the context of a polynucleotide of interest means presenting a nucleotide sequence of interest (e.g., polynucleotide, RT template, a nucleic acid construct, and/or a guide nucleic acid) to a plant, plant part thereof, or cell thereof, in such a manner that the nucleotide sequence gains access to the interior of a cell.

The terms “transformation” or transfection” may be used interchangeably and as used herein refer to the introduction of a heterologous nucleic acid into a cell. Transformation of a cell may be stable or transient. Thus, in some embodiments, a host cell or host organism (e.g., a plant) may be stably transformed with a polynucleotide/nucleic acid molecule of the invention. In some embodiments, a host cell or host organism may be transiently transformed with a polynucleotide/nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes comprising polynucleotides for editing as described herein) may be transiently introduced into a cell with a guide nucleic acid and as such, no DNA is maintained in the cell.

A nucleic acid construct of the invention may be introduced into a plant cell by any method known to those of skill in the art. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

In some embodiments of the invention, transformation of a cell may comprise nuclear transformation. In other embodiments, transformation of a cell may comprise plastid transformation (e.g., chloroplast transformation). In still further embodiments, nucleic acids of the invention may be introduced into a cell via conventional breeding techniques. In some embodiments, one or more of the polynucleotides, expression cassettes and/or vectors may be introduced into a plant cell via Agrobacterium transformation.

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

Plant flowering and architecture are mediated through a small group of regulatory factors with structural similarities to Phosphatidyl Ethanolamine Binding Proteins (PEBP). A naturally occurring variant in TFL1 in wild strawberry, for example, reduces flowering time (Plant Journal 69, 116-12 (2012)).

Individual TFL-like family members can be grouped into TFL1, CEN and BFT clades based on sequence identity and synteny. The dominant function of TFL1, CEN and BFT members is to regulate flowering time. However, there are distinct secondary functions associated with individual members that can be exploited for crop improvement. One major secondary function is the control of meristem determinacy. For example, modification of TFL1 is responsible for the self-pruning phenotype in tomato where the plant changes growth habit from indeterminate to determinate.

The ability to alter flowering timing and other related characteristics in a plant may be useful for breeding and production for both agricultural and horticultural crops. The present invention provides plants, in particular, plants in the family Rosaceae, having a reduced time to flowering (e.g., reduced time to flower initiation and fruit production), a longer duration of flowering, a more determinate plant growth pattern and/or one or more improved yield characteristics for use in production and breeding, as well as methods and compositions for producing such plants.

As described herein, Rosaceae plants having a reduced time to flowering (e.g., reduced time to flower initiation and fruit production), a longer duration of flowering, a more determinate plant growth pattern and/or one or more improved yield characteristics are generated using editing technology that targets TFL genes. The time to flowering in the Rosaceae plants (e.g., Rubus spp., Prunus spp., Fragaria spp., Malus spp., etc.) of this invention is reduced as compared to the unedited Rosaceae plants (e.g., a plant not comprising or devoid of the edit). In some aspects, a mutation will be a hypomorphic mutation, which may be advantageous if you wish flowering time to occur at a certain date. In some embodiments, mutations useful for producing such phenotypes may be generated by truncating or entirely deleting the TFL polypeptide. Such a mutation may be a null mutation, which is expected to have the most extreme early flowering phenotype. Other types of mutations useful for production of Rosaceae plants having a reduced time to flowering, a longer duration of flowering, a more determinate plant growth pattern and/or one or more improved yield characteristics (or any combination thereof) include substitutions, deletions and/or insertions.

In some embodiments, a Rosaceae plant or plant part thereof is provided, the Rosaceae plant or plant part thereof comprising at least one mutation (e.g., 1, 2, 3, 4, 5, or more mutations) in at least one copy of an endogenous gene encoding a TFL polypeptide (e.g., in 1, 2, 3, 4 or more copies). In some embodiments, a Rosaceae plant or plant part may comprise 1, 2, 3, or 4 TFL alleles and one or more (e.g., 1, 2, 3, or 4) may be mutated as described herein, optionally wherein all TFL alleles of the Rosaceae plant comprise one or more mutation as described herein. In some embodiments, at least one mutation in the Rosaceae plant or plant part may be a null allele. In some embodiments, at least one mutation in the Rosaceae plant or plant part may be a dominant-negative mutation, semi-dominant mutation, weak loss of function mutation, or a hypomorphic mutation. In some embodiments, at least one mutation in the Rosaceae plant or plant part may be a non-natural mutation. In some embodiments, the at least one mutation results in a mutated TFL1 gene having at least 90% sequence identity to any one of SEQ ID NO:277-284, 295, 297, 298, 300, 302, 304, 306, 308, 309, 310, 311, or 313 and/or encoding a mutated TFL1 polypeptide having at least 90% sequence identity to any one of SEQ ID NOs:285-291, 296, 299, 301, 303, 305, 307, or 312.

In some embodiments, a mutation in an endogenous TFL gene may provide Rosaceae plants having a more determinate growth habit as compared to a wild-type plant. Wild type Rosaceae plants such as plants in the Rubus genus may display a rambling type of growth habit in which the canes of the plant continue to grow throughout the life cycle of the plant. A plant of this invention would, for example, display a more compact, bushy growth habit.

In some embodiments, a Rosaceae plant cell, comprising an editing system comprising: (a) a CRISPR-Cas effector protein; and (b) a guide nucleic acid (gRNA, gDNA, crRNA, crDNA, sgRNA, sgDNA) comprising a spacer sequence with complementarity to an endogenous target gene encoding a TFL polypeptide in the Rosaceae plant cell. The editing system may be used to generate a mutation in the endogenous target gene encoding a TFL polypeptide. In some embodiments, the mutation may be a non-natural mutation. In some embodiments, a guide nucleic acid of an editing system may comprise the nucleotide sequence (a spacer sequence, e.g., one or more spacers) of any of one of SEQ ID NOs:198-210, 211-213 or 247-251.

In some embodiments, a Rosaceae plant cell comprising at least one (e.g., one or more) mutation within an endogenous TFL gene is provided, wherein the at least one mutation is a base substitution, base insertion or a base deletion that is introduced using an editing system that comprises a nucleic acid binding domain that binds to a target site in the endogenous TFL gene, optionally wherein the target site is within a region of the TFL gene, said region: comprising a sequence having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270 and/or encoding a sequence having at least 80% sequence identity to any one of the amino acid sequences of SEQ ID NOs:193-197, 247, 248, or 271-276. In some embodiments, wherein the editing system further comprise a nuclease, and the nucleic acid binding domain binds to a target site within a sequence having least 80% sequence identity to any one of the nucleotide sequences of SEQ ID NOs:72, 73, 106, 107, 114, 115, 126, 127, 140, 141, 153, 162, 163, 172, 173, 184, 185, 214, 215, 234, 235, or 252 and/or a sequence having at least 80% sequence identity to any one of the nucleotide sequence of SEQ ID NOs:75-105, 109-113, 117-125, 129-139, 143-152, 155-161, 165-171, 175-183, 187-192, 217-233, 237-246, or 254-270, and the at least one mutation is made following cleavage by the nuclease.

A mutation in a TFL gene of a Rosaceae plant, part thereof or Rosaceae plant cell useful for this invention may be any type of mutation, including a base substitution, a base deletion, and/or a base insertion, optionally wherein the mutation is a point mutation. In some embodiments, a mutation may be a non-natural mutation. In some embodiments, a mutation may comprise a base substitution to an A, a T, a G, or a C. In some embodiments, a mutation may be a deletion or insertion of at least one base pair (e.g., 1 base pair to about 100 base pairs, or about 3 base pairs to about 519 base pairs or about 3 base pairs to about 2600 base pairs and any range or value therein; e.g., about 1, 2, 3, 4, 5, 6, 7, 8, 9, 2, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 141, 142, 143, 144, 145, 150, 160, 170, 180, 190,200, 210, 220, 221, 222, 223, 224, 225, 230, 240, 250, 260, 270, 280, 290, 300, 310, 320, 340, 350, 360, 370, 380, 390, 400, 410, 420, 430, 440, 450, 460, 470, 480, 490, 500, 510, 515, 516, 517, 518, 519, 520, 530, 540, 550, 575, 600, 625, 650, 675, 700, 750, 800, 850, 900, 950, 1000, 1100, 1200, 1300, 1400, 1500, 1600, 1700, 1800, 1900, 2000, 2100, 2200, 2300, 2400, 2500, or 2600, and any range or value therein), optionally a deletion of about 5 to about 100 consecutive nucleotides.

In some embodiments, a mutation in an endogenous TFL gene of a Rosaceae plant or part thereof can be a deletion of at least 3 consecutive base pairs from the region of the TFL gene encoding a TFL polypeptide, wherein the TFL gene comprises a sequence having at least 80% sequence identity to any one of the nucleotide sequences of SEQ ID NOs:72, 73, 106, 107, 114, 115, 126, 127, 140, 141, 153, 162, 163, 172, 173, 184, 185, 214, 215, 234, 235, or 252, optionally wherein the deletion is about 3 consecutive base pairs to about 2600 consecutive base pairs, optionally about 5 consecutive base pairs to about 100 consecutive base pairs, of a sequence having at least 80% sequence identity to any one of the nucleotide sequences of SEQ ID NOs:72, 106, 114, 126, 140, 162, 172, 184, 214, 234 or 252 and/or about 3 consecutive base pairs to about 516 consecutive base pairs from a sequence having at least 80% sequence identity to any one of the nucleotide sequences of SEQ ID NOs: 73, 107, 115, 127, 141, 153, 163, 173, 185, 215 or 235. In some embodiments, the deletion in a TFL gene as described herein results in a C-terminal truncation of at least 1 amino acid residue from the C-terminus of a TFL polypeptide sequence having at least 80% sequence identity to the amino acid sequence of any one of SEQ ID NOs:74, 108, 116, 128, 142, 154, 164, 174, 186, 216, 236 or 253, optionally a deletion of about 1 amino acid residue to about 172 consecutive amino acid residues from any one of the amino acid sequences of SEQ ID NOs:74, 108, 116, 128, 142, 154, 164, 174, 186, 216, 236 or 253.

An endogenous TFL gene useful with this invention may be any TFL gene that encodes a TFL polypeptide involved in the flowering process of a Rosaceae plant, such as Rubus spp., Prunus spp, Fragaria spp., or Malus spp. In some embodiments, the endogenous TFL gene may be a TFL1 gene encoding a TFL1 polypeptide. In some embodiments, the endogenous target gene (e.g., endogenous TFL gene) to which a spacer sequence of the guide nucleic acid is complementary may: (a) comprise a nucleotide sequence having at least 80% sequence identity (e.g., about 80, 81, 82, 83, 84, 85, 86, 87, 88, 89, 90, 91, 92, 93, 94, 95, 96, 97, 98, 99, 99.5, 100% sequence identity; optionally about 90, 91, 92, 93, 94, 95, 96, 97, 98, 99, 99.5, 100% sequence identity or about 95, 96, 97, 98, 99, 99.5, or 100% sequence identity) to any one of SEQ ID NOs:72, 73, 106, 107, 114, 115, 126, 127, 140, 141, 153, 162, 163, 172, 173, 184, 185, 214, 215, 234, 235, or 252, (b) comprise a region having at least 80% sequence identity to any one of SEQ ID NOs:75-105, 109-113, 117-125, 129-139, 143-152, 155-161, 165-171, 175-183, 187-192, 217-233, 237-246, or 254-270, (c) encode a sequence having at least 80% sequence identity to any one of the amino acid sequences of SEQ ID NOs:74, 108, 116, 128, 142, 154, 164, 174, 186, 216, 236 or 253, and/or (d) encode a region having at least 80% sequence identity to any one of the amino acid sequences of SEQ ID NOs:193-197, 247, 248, or 271-276. In some embodiments, a spacer sequence of a guide nucleic acid of an editing system of this invention may comprise a nucleotide sequence of any one of SEQ ID NOs:198-210, 211-213 or 247-251.

In some embodiments, a Rosaceae plant or part thereof may comprise one or more endogenous TFL genes and one or more alleles of the one or more genes may each comprise a mutation as described herein.

The mutation in an endogenous TFL gene of a Rosaceae plant, plant part or plant cell may be any mutation as described herein, including a base deletion, base substitution or base insertion. In some embodiments, the at least one mutation may a dominant negative mutation, a semi-dominant mutation, a weak loss of function mutation, a null mutation, or a hypomorphic mutation, optionally, wherein the at least one mutation results in a null allele. In some embodiments, at least one mutation may be a non-natural mutation. In some embodiments, the mutation in a TFL gene may be a hypomorphic mutation and results in an amino acid substitution within the encoded TFL polypeptide. In some embodiments, the mutation may be a truncation or deletion (e.g., no detectable TFL protein) of the TFL gene and encoded TFL polypeptide (e.g., a null mutation, null allele or knockout). A mutation of an endogenous TFL gene of a Rosaceae plant, plant part or plant cell may result in the plant or plant regenerated from the plant part or plant cell having a reduced time to flowering, a longer duration of flowering, a more determinate plant growth pattern and/or one or more improved yield characteristics as compared to a Rosaceae plant not comprising the mutation in the TFL gene. In some embodiments, a mutation in an endogenous TFL gene may result in a mutated TFL gene having at least 90% sequence identity to any one of SEQ ID NO:277-284, 295, 297, 298, 300, 302, 304, 306, 308, 309, 310, 311, or 313 and/or a mutated TFL gene that encodes a mutated TFL1 polypeptide having at least 90% sequence identity to any one of SEQ ID NOs:285-291, 296, 299, 301, 303, 305, 307, or 312.

In some embodiments, the nucleic acid binding domain of an editing system useful with this invention may be from a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, a nuclease useful with the invention is a zinc finger nuclease, a transcription activator-like effector nuclease (TALEN), an endonuclease (e.g., Fok1) or a CRISPR-Cas effector protein. In some embodiments, a mutation of an endogenous TFL gene is made following cleavage by an editing system that comprises a nuclease and a nucleic acid binding domain that binds to a target site within a sequence having least 80% sequence identity to a sequence encoding of any one of SEQ ID NOs:72, 73, 106, 107, 114, 115, 126, 127, 140, 141, 153, 162, 163, 172, 173, 184, 185, 214, 215, 234, 235, or 252, or having at least 80% sequence identity to a sequence encoding any one of the amino acid sequences of SEQ ID NOs:74, 108, 116, 128, 142, 154, 164, 174, 186, 216, 236 or 253, and the at least one mutation within a TFL gene is made following cleavage by the nuclease, optionally wherein the mutation may be a non-natural mutation.

In some embodiments, a plant part/plant cell edited as described herein may be regenerated into a Rosaceae plant, thereby providing a Rosaceae plant with a mutation in a TFL gene that is involved in flowering time and having reduced time to flowering, a longer duration of flowering, a more determinate plant growth pattern and/or one or more improved yield characteristics (e.g., increased fruit production) as compared to a Rosaceae plant not comprising the mutation in the TFL gene. In some embodiments, the Rosaceae plant may comprise a mutated TFL gene having at least 90% sequence identity to any one of SEQ ID NO:277-284, 295, 297, 298, 300, 302, 304, 306, 308, 309, 310, 311, or 313 and/or encoding a mutated TFL1 polypeptide having at least 90% sequence identity to any one of SEQ ID NOs:285-291, 296, 299, 301, 303, 305, 307, or 312.

A Rosaceae plant, part thereof, or cell useful with this invention may be (or may be from) any caneberry plant or stone fruit plant, optionally a blackberry plant, a black raspberry plant, a cherry plant, a plum plant and/or a peach plant.

In some embodiments, a method of producing/breeding a transgene-free edited Rosaceae plant is provided, comprising: crossing a Rosaceae plant of the present invention (e.g., a Rosaceae plant comprising a mutation in a TFL protein/gene and exhibiting a reduced time to flowering, longer duration of flowering, a more determinate growth pattern and/or one or more improved yield traits) with a transgene free Rosaceae plant, thereby introducing the at least one mutation into the Rosaceae plant that is transgene-free; and selecting a progeny Rosaceae plant that comprises the at least one mutation and is transgene-free, thereby producing a transgene free edited Rosaceae plant, optionally the at least one mutation may be a non-natural mutation.

Also provided is a method of providing a plurality of Rosaceae plants having a reduced time to flowering, a longer duration of flowering, a more determinate plant growth pattern and/or one or more improved yield characteristics, the method comprising planting two or more Rosaceae plants of the invention (e.g., 2, 3, 4, 5, 6, 7, 8, 9, 10 or more Rosaceae plants comprising a mutation in a TFL protein/gene (including a deletion of coding sequence) and having a reduced time to flowering, a longer duration of flowering, a more determinate plant growth pattern and/or one or more improved yield characteristics) in a growing area (e.g., a field (e.g., a cultivated field, an agricultural field), a growth chamber, a greenhouse, a recreational area, a lawn, and/or a roadside and the like), thereby providing a plurality of Rosaceae plants having a reduced time to flowering, a longer duration of flowering, a more determinate plant growth pattern and/or one or more improved yield characteristics as compared to a plurality of control Rosaceae plants devoid of the mutation. In some embodiments, an improved yield trait or characteristic may be, for example, increased fruit production on a per plant or per hectare basis.

In some embodiments, a Rosaceae plant (or a plurality of Rosaceae plants of this invention) may be selfed and/or may be outcrossed with another Rosaceae plant.

In some embodiments, a method of creating a mutation in one or more endogenous TFL genes in a Rosaceae plant is provided, the method comprising (a) targeting a gene editing system to a portion of the one or more endogenous TFL genes the portion comprising a sequence having at least 90% sequence identity to any one of SEQ ID NOs:75-105, 109-113, 117-125, 129-139, 143-152, 155-161, 165-171, 175-183, 187-192, 217-233, 237-246, or 254-270; and (b) selecting a Rosaceae plant that comprises a modification located in a region of the one or more endogenous TFL genes having at least 90% sequence identity to any one of SEQ ID NOs:75-105, 109-113, 117-125, 129-139, 143-152, 155-161, 165-171, 175-183, 187-192, 217-233, 237-246, or 254-270, optionally wherein the mutation may result in a mutated TFL gene having at least 90% sequence identity to any one of SEQ ID NO:277-284, 295, 297, 298, 300, 302, 304, 306, 308, 309, 310, 311, or 313 and/or encoding a mutated TFL1 polypeptide having at least 90% sequence identity to any one of SEQ ID NOs:285-291, 296, 299, 301, 303, 305, 307, or 312.

Further provided is a method of generating variation in a region of a TFL polypeptide, the method comprising: introducing an editing system into a plant cell, wherein the editing system is targeted to a region of a TFL gene that encodes the region of the TFL polypeptide; and contacting the region of the TFL gene with the editing system, thereby introducing a mutation into the TFL gene and generating variation in the TFL polypeptide of the plant cell. In some embodiments, the TFL gene comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, or encodes a sequence having at least 80% sequence identity to any one of SEQ ID NOs:74, 108, 116, 128, 142, 154, 164, 174, 186, 216, 236 or 253, optionally wherein the region of the TFL gene comprises a nucleotide sequence having at least 80% sequence identity to any one of SEQ ID NOs:75-105, 109-113, 117-125, 129-139, 143-152, 155-161, 165-171, 175-183, 187-192, 217-233, 237-246, or 254-270, or encodes an amino acid sequence having at least 80% sequence identity to any one of SEQ ID NOs:193-197, 247, 248, or 271-276, and the mutation is made following cleavage by the editing system that comprises a nuclease and a nucleic acid binding domain that binds to a target site within a sequence having at least 80% sequence identity to any one of the nucleotide sequences of SEQ ID NOs:72, 73, 106, 107, 114, 115, 126, 127, 140, 141, 153, 162, 163, 172, 173, 184, 185, 214, 215, 234, 235, or 252 or SEQ ID NOs:75-105, 109-113, 117-125, 129-139, 143-152, 155-161, 165-171, 175-183, 187-192, 217-233, 237-246, or 254-270.

In some embodiments, a method for editing a specific site in the genome of a Rosaceae plant cell, the method comprising: cleaving, in a site specific manner, a target site within an endogenous TFL gene in the Rosaceae plant cell, wherein the endogenous TFL gene (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, thereby generating an edit in the endogenous TFL gene of the Rosaceae plant cell and producing a plant cell comprising the edit in the endogenous TFL gene. In some embodiments, the edit results in a mutation, including but not limited to a deletion, substitution, or insertion. In some embodiments, the edit may be a nucleotide substitution to an A, a T, a G, or a C. In some embodiments, the edit results in a non-natural mutation. In some embodiments, the edit may result in a null allele, a dominant negative mutation, a semi-dominant mutation, a weak loss of function mutation, a null mutation (knock-out), or a hypomorphic mutation. In some embodiments, the edit is a deletion that results in a truncation of the TFL polypeptide or no detectable TFL polypeptide as described herein (e.g., a null mutation or knock-out). In some embodiments, an edit results in variation in the TFL gene and/or TFL polypeptide as described herein. In some embodiments, the edit may result in a mutated TFL gene having at least 90% sequence identity to any one of SEQ ID NO:277-284, 295, 297, 298, 300, 302, 304, 306, 308, 309, 310, 311, or 313 and/or encoding a mutated TFL1 polypeptide having at least 90% sequence identity to any one of SEQ ID NOs:285-291, 296, 299, 301, 303, 305, 307, or 312.

In some embodiments, the method for editing may further comprise regenerating a Rosaceae plant from the Rosaceae plant cell comprising the edit in the endogenous TFL gene, thereby producing a Rosaceae plant comprising the edit in the endogenous TFL gene and having a phenotype of a reduced time to flowering, a longer duration of flowering, a more determinate plant growth pattern and/or one or more improved yield characteristics compared to a control Rosaceae plant that is devoid the edit, optionally wherein the Rosaceae plant comprises a mutated TFL gene having at least 90% sequence identity to any one of SEQ ID NO:277-284, 295, 297, 298, 300, 302, 304, 306, 308, 309, 310, 311, or 313 and/or encoding a mutated TFL1 polypeptide having at least 90% sequence identity to any one of SEQ ID NOs:285-291, 296, 299, 301, 303, 305, 307, or 312.

In some embodiments, a method for making a Rosaceae plant (e.g., Rubus spp. Prunus spp., Fragaria spp., Malus spp.) is provided, the method comprising: (a) contacting a population of Rosaceae plant cells comprising at least one endogenous TFL gene with a nuclease linked to a nucleic acid binding domain (e.g., an editing system) that binds to a target site in the at least one endogenous TFL gene, wherein the at least one endogenous TFL gene: (i) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (ii) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (iii) encodes an amino acid sequence having at least 80% sequence identity to any one of SEQ ID NOs:74, 108, 116, 128, 142, 154, 164, 174, 186, 216, 236 or 253, and/or (iv) encodes a region having at least 80% sequence identity to any one of the amino acid sequences of SEQ ID NOs:193-197, 247, 248, or 271-276; (b) selecting a Rosaceae plant cell from said population that comprises a mutation in the at least one endogenous TFL gene; and (c) growing the selected Rosaceae plant cell into a Rosaceae plant, optionally wherein the mutation in the at least one endogenous TFL gene results in a null allele of the endogenous TFL gene. In some embodiments, the mutation in the at least one endogenous TFL gene may be a non-natural mutation.

In some embodiments, a method is provided for reducing time to flowering in a Rosaceae plant or part thereof, comprising (a) contacting a Rosaceae plant cell comprising an endogenous TFL gene with a nuclease targeting the endogenous TFL gene, wherein the nuclease is linked to a nucleic acid binding domain that binds to a target site in the endogenous TFL gene, wherein the endogenous TFL gene: (i) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (ii) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (iii) encodes an amino acid sequence having at least 80% sequence identity to any one of SEQ ID NOs:74, 108, 116, 128, 142, 154, 164, 174, 186, 216, 236 or 253, and/or (iv) encodes a region having at least 80% sequence identity to any one of the amino acid sequences of SEQ ID NOs:193-197, 247, 248, or 271-276; and (b) growing the Rosaceae plant cell into a Rosaceae plant comprising a mutation in the endogenous TFL gene, thereby reducing time to flowering in the Rosaceae plant or part thereof. In some embodiments, the mutation in the at least one endogenous TFL gene may be a non-natural mutation.

In some embodiments, a method is provided for lengthening (increasing) the duration of flowering in a Rosaceae plant or part thereof, comprising (a) contacting a Rosaceae plant cell comprising an endogenous TFL gene with a nuclease targeting the endogenous TFL gene, wherein the nuclease is linked to a nucleic acid binding domain that binds to a target site in the endogenous TFL gene, wherein the endogenous TFL gene: (i) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (ii) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (iii) encodes an amino acid sequence having at least 80% sequence identity to any one of SEQ ID NOs:74, 108, 116, 128, 142, 154, 164, 174, 186, 216, 236 or 253, and/or (iv) encodes a region having at least 80% sequence identity to any one of the amino acid sequences of SEQ ID NOs:193-197, 247, 248, or 271-276; and (b) growing the Rosaceae plant cell into a Rosaceae plant comprising a mutation in the endogenous TFL gene, thereby lengthening (increasing) the duration of flowering in the Rosaceae plant or part thereof.

In some embodiments, a method for providing a more determinate plant growth pattern in a Rosaceae plant or part thereof is provided, comprising (a) contacting a Rosaceae plant cell comprising an endogenous TFL gene with a nuclease targeting the endogenous TFL gene, wherein the nuclease is linked to a nucleic acid binding domain that binds to a target site in the endogenous TFL gene, wherein the endogenous TFL gene: (i) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (ii) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (iii) encodes an amino acid sequence having at least 80% sequence identity to any one of SEQ ID NOs:74, 108, 116, 128, 142, 154, 164, 174, 186, 216, 236 or 253, and/or (iv) encodes a region having at least 80% sequence identity to any one of the amino acid sequences of SEQ ID NOs:193-197, 247, 248, or 271-276; and (b) growing the Rosaceae plant cell into a Rosaceae plant comprising a mutation in the endogenous TFL gene, thereby providing the Rosaceae plant or part thereof having a more determinate growth pattern.

In some embodiments, a method for providing a Rosaceae plant or part thereof having one or more improved yield characteristics is provided, comprising (a) contacting a Rosaceae plant cell comprising an endogenous TFL gene with a nuclease targeting the endogenous TFL gene, wherein the nuclease is linked to a nucleic acid binding domain that binds to a target site in the endogenous TFL gene, wherein the endogenous TFL gene: (i) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (ii) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (iii) encodes an amino acid sequence having at least 80% sequence identity to any one of SEQ ID NOs:74, 108, 116, 128, 142, 154, 164, 174, 186, 216, 236 or 253, and/or (iv) encodes a region having at least 80% sequence identity to any one of the amino acid sequences of SEQ ID NOs:193-197, 247, 248, or 271-276; and (b) growing the Rosaceae plant cell into a Rosaceae plant comprising a mutation in the endogenous TFL gene, thereby providing the Rosaceae plant or part thereof having one or more improved yield characteristics. In some embodiments, an improved yield trait or characteristic may be, for example, increased fruit production on a per plant or per hectare basis.

In some embodiments, a Rosaceae plant or part thereof is provided that exhibits a phenotype of any combination of a reduced time to flowering, a longer duration of flowering, a more determinate plant growth pattern and/or one or more improved yield characteristics.

In some embodiments, a method is provided for producing a Rosaceae plant or part thereof comprising at least one cell having an endogenous TFL gene with a mutation, the method comprising contacting a target site in an endogenous TFL gene in the Rosaceae plant or part with a nuclease comprising a cleavage domain and a DNA-binding domain, wherein the nucleic acid binding domain binds to a target site in the endogenous TFL gene, wherein the endogenous TFL gene (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, thereby producing the Rosaceae plant or part thereof comprising at least one cell having an endogenous TFL gene with a mutation.

Also provided herein is a method for producing a Rosaceae plant or part thereof comprising a mutated endogenous TFL gene and exhibiting a phenotype of reduced time to flowering, a longer duration of flowering, a more determinate plant growth pattern and/or one or more improved yield characteristics (in an combination), the method comprising contacting a target site in an endogenous TFL gene in the Rosaceae plant or part thereof with a nuclease comprising a cleavage domain and a DNA-binding domain, wherein the nucleic acid binding domain binds to a target site in the endogenous TFL gene, wherein the endogenous TFL gene: (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, thereby producing the Rosaceae plant or part thereof comprising an endogenous TFL gene having a mutation and exhibiting a phenotype of reduced time to flowering, a longer duration of flowering, a more determinate plant growth pattern and/or one or more improved yield characteristics.

In some embodiments, a nuclease may cleave an endogenous TFL gene, thereby introducing the mutation into the endogenous TFL gene. A nuclease useful with the invention may be any nuclease that can be utilized to edit/modify a target nucleic acid. Such nucleases include, but are not limited to a zinc finger nuclease, transcription activator-like effector nucleases (TALEN), endonuclease (e.g., Fok1) and/or a CRISPR-Cas effector protein. Likewise, any nucleic acid binding domain useful with the invention may be any DNA binding domain or RNA binding domain that can be utilized to edit/modify a target nucleic acid. Such nucleic acid binding domains include, but are not limited to, a zinc finger, transcription activator-like DNA binding domain (TAL), an argonaute and/or a CRISPR-Cas effector DNA binding domain.

In some embodiments, a nucleic acid binding domain (e.g., DNA binding domain) is comprised in a nucleic acid binding polypeptide. A “nucleic acid binding protein” or “nucleic acid binding polypeptide” as used herein refers to a polypeptide that binds and/or is capable of binding a nucleic acid in a site- and/or sequence-specific manner. In some embodiments, a nucleic acid binding polypeptide may be a sequence-specific nucleic acid binding polypeptide (e.g., a sequence-specific DNA binding domain) such as, but not limited to, a sequence-specific binding polypeptide and/or domain from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, a nucleic acid binding polypeptide comprises a cleavage polypeptide (e.g., a nuclease polypeptide and/or domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, the nucleic acid binding polypeptide associates with and/or is capable of associating with (e.g., forms a complex with) one or more nucleic acid molecule(s) (e.g., forms a complex with a guide nucleic acid as described herein) that can direct or guide the nucleic acid binding polypeptide to a specific target nucleotide sequence (e.g., a gene locus of a genome) that is complementary to the one or more nucleic acid molecule(s) (or a portion or region thereof), thereby causing the nucleic acid binding polypeptide to bind to the nucleotide sequence at the specific target site. In some embodiments, the nucleic acid binding polypeptide is a CRISPR-Cas effector protein as described herein. In some embodiments, reference is made to specifically to a CRISPR-Cas effector protein for simplicity, but a nucleic acid binding polypeptide as described herein may be used. In some embodiments, a polynucleotide and/or a nucleic acid construct of the invention can be an “expression cassette” or can be comprised within an expression cassette.

In some embodiments, a method of editing an endogenous TFL gene in a plant or plant part is provided, the method comprising contacting a target site in the TFL gene in the plant or plant part with a cytosine base editing system comprising a cytosine deaminase and a nucleic acid binding domain that binds to a target site in the TFL gene, the TFL gene (a) comprising a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprising a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encoding an amino acid sequence having at least 80% sequence identity to any one of SEQ ID NOs:74, 108, 116, 128, 142, 154, 164, 174, 186, 216, 236 or 253, and/or (d) encoding a region having at least 80% sequence identity to any one of the amino acid sequences of SEQ ID NOs:193-197, 247, 248, or 271-276, thereby editing the endogenous TFL gene in the plant or part thereof and producing a plant or part thereof comprising at least one cell having a mutation in the endogenous TFL gene.

In some embodiments, a method of editing an endogenous TFL gene in a plant or plant part is provided, the method comprising contacting a target site in the TFL gene in the plant or plant part with an adenosine base editing system comprising an adenosine deaminase and a nucleic acid binding domain that binds to a target site in the TFL gene, the TFL gene (a) comprising a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprising a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encoding an amino acid sequence having at least 80% sequence identity to any one of SEQ ID NOs:74, 108, 116, 128, 142, 154, 164, 174, 186, 216, 236 or 253, and/or (d) encoding a region having at least 80% sequence identity to any one of the amino acid sequences of SEQ ID NOs:193-197, 247, 248, or 271-276, thereby editing the endogenous TFL gene in the plant or part thereof and producing a plant or part thereof comprising at least one cell having a mutation in the endogenous TFL gene.

A mutation provided by methods of the invention may be a substitution, an insertion and/or a deletion, optionally wherein the insertion or deletion is a frameshift mutation, e.g., an in-frame insertion or in-frame deletion. In some embodiments, the mutation may be a deletion of about 1 base pair, optionally about 3 consecutive base pairs to about 519 consecutive base pairs or about 3 consecutive base pairs to about 2600 consecutive base pairs, or any value or range therein. In some embodiments, the mutation may be a non-natural mutation.

In some embodiments, a method of detecting a mutant TFL gene (a mutation in an endogenous TFL gene) is provide, the method comprising detecting in the genome of a Rosaceae plant a deletion in a nucleic acid (a) comprising a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprising a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encoding an amino acid sequence having at least 80% sequence identity to any one of SEQ ID NOs:74, 108, 116, 128, 142, 154, 164, 174, 186, 216, 236 or 253, and/or (d) encoding a region having at least 80% sequence identity to any one of the amino acid sequences of SEQ ID NOs:193-197, 247, 248, or 271-276. In some embodiments, the deletion results in a truncation or deletion of about 10 consecutive amino acid residues to about 172 consecutive amino acid residues of any one of the amino acid sequences of SEQ ID NOs:74, 108, 116, 128, 142, 154, 164, 174, 186, 216, 236 or 253, optionally a deletion of at least one residue in a region of any one of amino acid sequences of SEQ ID NOs:74, 108, 116, 128, 142, 154, 164, 174, 186, 216, 236 or 253 (e.g., a region having at least 80% sequence identity to any one of the amino acid sequences of SEQ ID NOs:193-197, 247, 248, or 271-276).

In some embodiments, the present invention provides a method of detecting a mutation in an endogenous TFL gene, comprising detecting in the genome of a plant a mutated TFL gene produced as described herein. In some embodiments, a mutant TFL gene that is detected comprises a nucleic acid sequence having at least 90% sequence identity to any one of SEQ ID NO:277-284, 295, 297, 298, 300, 302, 304, 306, 308, 309, 310, 311, or 313 and/or encodes a mutated TFL polypeptide having at least 90% sequence identity to any one of SEQ ID NOs:285-291, 296, 299, 301, 303, 305, 307, or 312.

In another aspect, Rosaceae plants or plant part produced by the methods of the invention are provided that comprise in their genome one or more mutated endogenous TFL genes as described herein. In some embodiments, the methods of the invention produce Rosaceae plants or parts thereof having a mutated endogenous TLF gene having a at least 90% sequence identity to any one of SEQ ID NO:277-284, 295, 297, 298, 300, 302, 304, 306, 308, 309, 310, 311, or 313 and/or encode a mutated TFL1 polypeptide having at least 90% sequence identity to any one of SEQ ID NOs:285-291, 296, 299, 301, 303, 305, 307, or 312.

Another aspect of the invention provides a nucleic acid comprising a mutated TFL gene, wherein the mutated TFL gene when expressed produces a truncated TFL polypeptide and/or no detectable TFL polypeptide. A further aspect provides a mutated nucleic acid encoding a TFL polypeptide, optionally the mutation is comprised in the coding region of the TFL gene, wherein the mutation results in a truncated TFL polypeptide or no detectable TFL polypeptide.

In some embodiments, a mutation in an endogenous TLF gene may result in a nucleic acid sequence having a at least 90% sequence identity to any one of SEQ ID NO:277-284, 295, 297, 298, 300, 302, 304, 306, 308, 309, 310, 311, or 313 and/or encode a mutated TFL1 polypeptide having at least 90% sequence identity to any one of SEQ ID NOs:285-291, 296, 299, 301, 303, 305, 307, or 312. In some embodiments, the mutated endogenous TFL gene comprises a non-natural mutation.

Further provided are Rosaceae plants (e.g., Prunus plants, Rubus plants, Fragaria plants, Malus plants, etc.) comprising in their genome one or more TFL genes having a mutation produced by the methods of the invention, optionally wherein the Rosaceae plants exhibit a phenotype of reduced time to flowering, a longer duration of flowering, a more determinate plant growth pattern and/or one or more improved yield characteristics, or any combination thereof, as compared to a Rosaceae plant not comprising the mutation. In some embodiments, the mutation may be a non-natural mutation. In some embodiments, the mutation in an endogenous TLF gene may result in a nucleic acid sequence having a at least 90% sequence identity to any one of SEQ ID NO:277-284, 295, 297, 298, 300, 302, 304, 306, 308, 309, 310, 311, or 313 and/or encode a mutated TFL1 polypeptide having at least 90% sequence identity to any one of SEQ ID NOs:285-291, 296, 299, 301, 303, 305, 307, or 312. In some embodiments, the mutated endogenous TFL gene comprises a non-natural mutation.

In some embodiments, the present invention provides a method of producing a Rosaceae plant comprising a mutation in an endogenous TFL gene and at least one polynucleotide of interest, the method comprising crossing a Rosaceae plant of the invention comprising at least one mutation in an endogenous TFL gene (a first Rosaceae plant) with a second Rosaceae plant that comprises the at least one polynucleotide of interest to produce progeny plants; and selecting progeny plants comprising at least one mutation in the TFL gene and the at least one polynucleotide of interest, thereby producing the Rosaceae plant comprising a mutation in an endogenous TFL gene and at least one polynucleotide of interest.

The present invention further provides a method of producing a Rosaceae plant comprising a mutation in an endogenous TFL gene and at least one polynucleotide of interest, the method comprising introducing at least one polynucleotide of interest into a Rosaceae plant of the present invention comprising at least one mutation in a TFL gene, thereby producing the Rosaceae plant comprising at least one mutation in a TFL gene and at least one polynucleotide of interest.

In some embodiments, the present invention provides a method of producing a Rosaceae plant comprising a mutation in an endogenous TFL gene and at least one polynucleotide of interest, the method comprising introducing at least one polynucleotide of interest into a Rosaceae plant of the invention comprising at least one mutation in an endogenous TFL gene, thereby producing the Rosaceae plant comprising at least one mutation in a TFL gene and at least one polynucleotide of interest.

In some embodiments, also provided is a method of producing a Rosaceae plant comprising a mutation in an endogenous TFL gene and exhibiting a phenotype of improved plant architecture and/or improved defense traits, the method comprising crossing a first Rosaceae plant, which is a Rosaceae plant of the present invention comprising at least one mutation in a TFL gene, with a second Rosaceae plant that exhibits a phenotype of improved plant architecture and/or improved defense traits; and selecting progeny plants comprising the mutation in the TFL gene and a phenotype of improved plant architecture and/or improved defense traits, thereby producing the Rosaceae plant comprising a mutation in an endogenous TFL gene and exhibiting a phenotype of improved plant architecture and/or improved defense traits as compared to a control Rosaceae plant.

Further provided is a method of controlling weeds in a container (e.g., pot, or seed tray and the like), a growth chamber, a greenhouse, a field, a recreational area, a lawn, or on a roadside, the method comprising applying an herbicide to one or more (a plurality) Rosaceae plants of the invention growing in a container, a growth chamber, a greenhouse, a field, a recreational area, a lawn, or on a roadside, thereby controlling the weeds in the container, the growth chamber, the greenhouse, the field, the recreational area, the lawn, or on the roadside in which the one or more Rosaceae plants are growing.

In some embodiments, a method of reducing insect predation on a plant is provided, the method comprising applying an insecticide to one or more Rosaceae plants of the invention, optionally, wherein the one or more Rosaceae plants are growing in a container, a growth chamber, a greenhouse, a field, a recreational area, a lawn, or on a roadside, thereby reducing insect predation on the one or more Rosaceae plants.

In some embodiments, a method of reducing fungal disease on a plant is provided, the method comprising applying a fungicide to one or more Rosaceae plants of the invention, optionally, wherein the one or more Rosaceae plants are growing in a container, a growth chamber, a greenhouse, a field, a recreational area, a lawn, or on a roadside, thereby reducing fungal disease on the one or more Rosaceae plants.

A polynucleotide of interest may be any polynucleotide that can confer a desirable phenotype or otherwise modify the phenotype or genotype of a plant. In some embodiments, a polynucleotide of interest may be polynucleotide that confers herbicide tolerance, insect resistance, nematode resistance, disease resistance, increased yield, increased nutrient use efficiency or abiotic stress resistance.

Thus, plants or plant cultivars which are to be treated with preference in accordance with the invention include all plants which, through genetic modification, received genetic material which imparts particular advantageous useful properties (“traits”) to these plants. Examples of such properties are better plant growth, vigor, stress tolerance, standability, lodging resistance, nutrient uptake, plant nutrition, and/or yield, in particular improved growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processability of the harvested products.

Further examples of such properties are an increased resistance against animal and microbial pests, such as against insects, arachnids, nematodes, mites, slugs and snails owing, for example, to toxins formed in the plants. Among DNA sequences encoding proteins which confer properties of tolerance to such animal and microbial pests, in particular insects, mention will particularly be made of the genetic material from Bacillus thuringiensis encoding the Bt proteins widely described in the literature and well known to those skilled in the art. Mention will also be made of proteins extracted from bacteria such as Photorhabdus (WO97/17432 and WO98/08932). In particular, mention will be made of the Bt Cry or VIP proteins which include the Cry1A, CryIAb, CryIAc, CryIIA, CryIIIA, CryIIIB2, Cry9c Cry2Ab, Cry3Bb and CryIF proteins or toxic fragments thereof and also hybrids or combinations thereof, especially the Cry1F protein or hybrids derived from a Cry1F protein (e.g. hybrid Cry1A-Cry1F proteins or toxic fragments thereof), the Cry1A-type proteins or toxic fragments thereof, preferably the Cry1Ac protein or hybrids derived from the Cry1Ac protein (e.g. hybrid Cry1Ab-Cry1Ac proteins) or the Cry1Ab or Bt2 protein or toxic fragments thereof, the Cry2Ae, Cry2Af or Cry2Ag proteins or toxic fragments thereof, the Cry1A.105 protein or a toxic fragment thereof, the VIP3Aa19 protein, the VIP3Aa20 protein, the VIP3A proteins produced in the COT202 or COT203 cotton events, the VIP3Aa protein or a toxic fragment thereof as described in Estruch et al. (1996), Proc Natl Acad Sci US A. 28; 93(11):5389-94, the Cry proteins as described in WO2001/47952, the insecticidal proteins from Xenorhabdus (as described in WO98/50427), Serratia (particularly from S. entomophila) or Photorhabdus species strains, such as Tc-proteins from Photorhabdus as described in WO98/08932. Also any variants or mutants of any one of these proteins differing in some amino acids (1-10, preferably 1-5) from any of the above named sequences, particularly the sequence of their toxic fragment, or which are fused to a transit peptide, such as a plastid transit peptide, or another protein or peptide, is included herein.

Another and particularly emphasized example of such properties is conferred tolerance to one or more herbicides, for example imidazolinones, sulphonylureas, glyphosate or phosphinothricin. Among DNA sequences encoding proteins (i.e., polynucleotides of interest) which confer properties of tolerance to certain herbicides on the transformed plant cells and plants, mention will be particularly be made to the bar or PAT gene or the Streptomyces coelicolor gene described in WO2009/152359 which confers tolerance to glufosinate herbicides, a gene encoding a suitable EPSPS (5-Enolpyruvylshikimat-3-phosphat-Synthase) which confers tolerance to herbicides having EPSPS as a target, especially herbicides such as glyphosate and its salts, a gene encoding glyphosate-n-acetyltransferase, or a gene encoding glyphosate oxidoreductase. Further suitable herbicide tolerance traits include at least one ALS (acetolactate synthase) inhibitor (e.g. WO2007/024782), a mutated Arabidopsis ALS/AHAS gene (e.g. U.S. Pat. No. 6,855,533), genes encoding 2,4-D-monooxygenases conferring tolerance to 2,4-D (2,4-dichlorophenoxyacetic acid) and genes encoding Dicamba monooxygenases conferring tolerance to dicamba (3,6-dichloro-2-methoxybenzoic acid).

Further examples of such properties are increased resistance against phytopathogenic fungi, bacteria and/or viruses owing, for example, to systemic acquired resistance (SAR), systemin, phytoalexins, elicitors and also resistance genes and correspondingly expressed proteins and toxins.

Particularly useful transgenic events in transgenic plants or plant cultivars which can be treated with preference in accordance with the invention include Event 531/PV-GHBK04 (cotton, insect control, described in WO2002/040677), Event 1143-14A (cotton, insect control, not deposited, described in WO2006/128569); Event 1143-51B (cotton, insect control, not deposited, described in WO2006/128570); Event 1445 (cotton, herbicide tolerance, not deposited, described in US-A 2002-120964 or WO2002/034946); Event 17053 (rice, herbicide tolerance, deposited as PTA-9843, described in WO2010/117737); Event 17314 (rice, herbicide tolerance, deposited as PTA-9844, described in WO2010/117735); Event 281-24-236 (cotton, insect control—herbicide tolerance, deposited as PTA-6233, described in WO2005/103266 or US-A 2005-216969); Event 3006-210-23 (cotton, insect control—herbicide tolerance, deposited as PTA-6233, described in US-A 2007-143876 or WO2005/103266); Event 3272 (corn, quality trait, deposited as PTA-9972, described in WO2006/098952 or US-A 2006-230473); Event 33391 (wheat, herbicide tolerance, deposited as PTA-2347, described in WO2002/027004), Event 40416 (corn, insect control-herbicide tolerance, deposited as ATCC PTA-11508, described in WO 11/075593); Event 43A47 (corn, insect control—herbicide tolerance, deposited as ATCC PTA-11509, described in WO2011/075595); Event 5307 (corn, insect control, deposited as ATCC PTA-9561, described in WO2010/077816); Event ASR-368 (bent grass, herbicide tolerance, deposited as ATCC PTA-4816, described in US-A 2006-162007 or WO2004/053062); Event B16 (corn, herbicide tolerance, not deposited, described in US-A 2003-126634); Event BPS-CV127-9 (soybean, herbicide tolerance, deposited as NCIMB No. 41603, described in WO2010/080829); Event BLR1 (oilseed rape, restoration of male sterility, deposited as NCIMB 41193, described in WO2005/074671), Event CE43-67B (cotton, insect control, deposited as DSM ACC2724, described in US-A 2009-217423 or WO2006/128573); Event CE44-69D (cotton, insect control, not deposited, described in US-A 2010-0024077); Event CE44-69D (cotton, insect control, not deposited, described in WO2006/128571); Event CE46-02A (cotton, insect control, not deposited, described in WO2006/128572); Event COT102 (cotton, insect control, not deposited, described in US-A 2006-130175 or WO2004/039986); Event COT202 (cotton, insect control, not deposited, described in US-A 2007-067868 or WO2005/054479); Event COT203 (cotton, insect control, not deposited, described in WO2005/054480);); Event DAS21606-3/1606 (soybean, herbicide tolerance, deposited as PTA-11028, described in WO2012/033794), Event DAS40278 (corn, herbicide tolerance, deposited as ATCC PTA-10244, described in WO2011/022469); Event DAS-44406-6/pDAB8264.44.06.1 (soybean, herbicide tolerance, deposited as PTA-11336, described in WO2012/075426), Event DAS-14536-7/pDAB8291.45.36.2 (soybean, herbicide tolerance, deposited as PTA-11335, described in WO2012/075429), Event DAS-59122-7 (corn, insect control—herbicide tolerance, deposited as ATCC PTA 11384, described in US-A 2006-070139); Event DAS-59132 (corn, insect control—herbicide tolerance, not deposited, described in WO2009/100188); Event DAS68416 (soybean, herbicide tolerance, deposited as ATCC PTA-10442, described in WO2011/066384 or WO2011/066360); Event DP-098140-6 (corn, herbicide tolerance, deposited as ATCC PTA-8296, described in US-A 2009-137395 or WO 08/112019); Event DP-305423-1 (soybean, quality trait, not deposited, described in US-A 2008-312082 or WO2008/054747); Event DP-32138-1 (corn, hybridization system, deposited as ATCC PTA-9158, described in US-A 2009-0210970 or WO2009/103049); Event DP-356043-5 (soybean, herbicide tolerance, deposited as ATCC PTA-8287, described in US-A 2010-0184079 or WO2008/002872); Event EE-I (brinjal, insect control, not deposited, described in WO 07/091277); Event Fil 17 (corn, herbicide tolerance, deposited as ATCC 209031, described in US-A 2006-059581 or WO 98/044140); Event FG72 (soybean, herbicide tolerance, deposited as PTA-11041, described in WO2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO98/044140); Event GHB119 (cotton, insect control—herbicide tolerance, deposited as ATCC PTA-8398, described in WO2008/151780); Event GHB614 (cotton, herbicide tolerance, deposited as ATCC PTA-6878, described in US-A 2010-050282 or WO2007/017186); Event GJ11 (corn, herbicide tolerance, deposited as ATCC 209030, described in US-A 2005-188434 or WO98/044140); Event GM RZ13 (sugar beet, virus resistance, deposited as NCIMB-41601, described in WO2010/076212); Event H7-1 (sugar beet, herbicide tolerance, deposited as NCIMB 41158 or NCIMB 41159, described in US-A 2004-172669 or WO 2004/074492); Event JOPLIN1 (wheat, disease tolerance, not deposited, described in US-A 2008-064032); Event LL27 (soybean, herbicide tolerance, deposited as NCIMB41658, described in WO2006/108674 or US-A 2008-320616); Event LL55 (soybean, herbicide tolerance, deposited as NCIMB 41660, described in WO 2006/108675 or US-A 2008-196127); Event LLcotton25 (cotton, herbicide tolerance, deposited as ATCC PTA-3343, described in WO2003/013224 or US-A 2003-097687); Event LLRICE06 (rice, herbicide tolerance, deposited as ATCC 203353, described in U.S. Pat. No. 6,468,747 or WO2000/026345); Event LLRice62 (rice, herbicide tolerance, deposited as ATCC 203352, described in WO2000/026345), Event LLRICE601 (rice, herbicide tolerance, deposited as ATCC PTA-2600, described in US-A 2008-2289060 or WO2000/026356); Event LY038 (corn, quality trait, deposited as ATCC PTA-5623, described in US-A 2007-028322 or WO2005/061720); Event MIR162 (corn, insect control, deposited as PTA-8166, described in US-A 2009-300784 or WO2007/142840); Event MIR604 (corn, insect control, not deposited, described in US-A 2008-167456 or WO2005/103301); Event MON15985 (cotton, insect control, deposited as ATCC PTA-2516, described in US-A 2004-250317 or WO2002/100163); Event MON810 (corn, insect control, not deposited, described in US-A 2002-102582); Event MON863 (corn, insect control, deposited as ATCC PTA-2605, described in WO2004/011601 or US-A 2006-095986); Event MON87427 (corn, pollination control, deposited as ATCC PTA-7899, described in WO2011/062904); Event MON87460 (corn, stress tolerance, deposited as ATCC PTA-8910, described in WO2009/111263 or US-A 2011-0138504); Event MON87701 (soybean, insect control, deposited as ATCC PTA-8194, described in US-A 2009-130071 or WO2009/064652); Event MON87705 (soybean, quality trait—herbicide tolerance, deposited as ATCC PTA-9241, described in US-A 2010-0080887 or WO2010/037016); Event MON87708 (soybean, herbicide tolerance, deposited as ATCC PTA-9670, described in WO2011/034704); Event MON87712 (soybean, yield, deposited as PTA-10296, described in WO2012/051199), Event MON87754 (soybean, quality trait, deposited as ATCC PTA-9385, described in WO2010/024976); Event MON87769 (soybean, quality trait, deposited as ATCC PTA-8911, described in US-A 2011-0067141 or WO2009/102873); Event MON88017 (corn, insect control—herbicide tolerance, deposited as ATCC PTA-5582, described in US-A 2008-028482 or WO2005/059103); Event MON88913 (cotton, herbicide tolerance, deposited as ATCC PTA-4854, described in WO2004/072235 or US-A 2006-059590); Event MON88302 (oilseed rape, herbicide tolerance, deposited as PTA-10955, described in WO2011/153186), Event MON88701 (cotton, herbicide tolerance, deposited as PTA-11754, described in WO2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO2006/130436); Event MSl 1 (oilseed rape, pollination control—herbicide tolerance, deposited as ATCC PTA-850 or PTA-2485, described in WO2001/031042); Event MS8 (oilseed rape, pollination control—herbicide tolerance, deposited as ATCC PTA-730, described in WO2001/041558 or US-A 2003-188347); Event NK603 (corn, herbicide tolerance, deposited as ATCC PTA-2478, described in US-A 2007-292854); Event PE-7 (rice, insect control, not deposited, described in WO2008/114282); Event RF3 (oilseed rape, pollination control—herbicide tolerance, deposited as ATCC PTA-730, described in WO2001/041558 or US-A 2003-188347); Event RT73 (oilseed rape, herbicide tolerance, not deposited, described in WO2002/036831 or US-A 2008-070260); Event SYHTOH2/SYN-000H2-5 (soybean, herbicide tolerance, deposited as PTA-11226, described in WO2012/082548), Event T227-1 (sugar beet, herbicide tolerance, not deposited, described in WO2002/44407 or US-A 2009-265817); Event T25 (corn, herbicide tolerance, not deposited, described in US-A 2001-029014 or WO2001/051654); Event T304-40 (cotton, insect control—herbicide tolerance, deposited as ATCC PTA-8171, described in US-A 2010-077501 or WO2008/122406); Event T342-142 (cotton, insect control, not deposited, described in WO2006/128568); Event TC1507 (corn, insect control—herbicide tolerance, not deposited, described in US-A 2005-039226 or WO2004/099447); Event VIP1034 (corn, insect control—herbicide tolerance, deposited as ATCC PTA-3925, described in WO2003/052073), Event 32316 (corn, insect control-herbicide tolerance, deposited as PTA-11507, described in WO2011/084632), Event 4114 (corn, insect control—herbicide tolerance, deposited as PTA-11506, described in WO2011/084621), event EE-GM3/FG72 (soybean, herbicide tolerance, ATCC Accession N° PTA-11041) optionally stacked with event EE-GM1/LL27 or event EE-GM2/LL55 (WO2011/063413A2), event DAS-68416-4 (soybean, herbicide tolerance, ATCC Accession No. PTA-10442, WO2011/066360A1), event DAS-68416-4 (soybean, herbicide tolerance, ATCC Accession No. PTA-10442, WO2011/066384A1), event DP-040416-8 (corn, insect control, ATCC Accession No. PTA-11508, WO2011/075593A1), event DP-043A47-3 (corn, insect control, ATCC Accession No. PTA-11509, WO2011/075595A1), event DP-004114-3 (corn, insect control, ATCC Accession No. PTA-11506, WO2011/084621A1), event DP-032316-8 (corn, insect control, ATCC Accession No. PTA-11507, WO2011/084632A1), event MON-88302-9 (oilseed rape, herbicide tolerance, ATCC Accession No. PTA-10955, WO2011/153186A1), event DAS-21606-3 (soybean, herbicide tolerance, ATCC Accession No. PTA-11028, WO2012/033794A2), event MON-87712-4 (soybean, quality trait, ATCC Accession No. PTA-10296, WO2012/051199A2), event DAS-44406-6 (soybean, stacked herbicide tolerance, ATCC Accession No. PTA-11336, WO2012/075426A1), event DAS-14536-7 (soybean, stacked herbicide tolerance, ATCC Accession No. PTA-11335, WO2012/075429A1), event SYN-000H2-5 (soybean, herbicide tolerance, ATCC Accession No. PTA-11226, WO2012/082548A2), event DP-061061-7 (oilseed rape, herbicide tolerance, no deposit No. available, WO2012071039A1), event DP-073496-4 (oilseed rape, herbicide tolerance, no deposit No. available, US2012131692), event 8264.44.06.1 (soybean, stacked herbicide tolerance, Accession No. PTA-11336, WO2012075426A2), event 8291.45.36.2 (soybean, stacked herbicide tolerance, Accession No. PTA-11335, WO2012075429A2), event SYHTOH2 (soybean, ATCC Accession No. PTA-11226, WO2012/082548A2), event MON88701 (cotton, ATCC Accession No. PTA-11754, WO2012/134808A1), event KK179-2 (alfalfa, ATCC Accession No. PTA-11833, WO2013/003558A1), event pDAB8264.42.32.1 (soybean, stacked herbicide tolerance, ATCC Accession No. PTA-11993, WO2013/010094A1), event MZDT09Y (corn, ATCC Accession No. PTA-13025, WO2013/012775A1).

The genes/events (e.g., polynucleotides of interest), which impart the desired traits in question, may also be present in combinations with one another in the transgenic plants. Examples of transgenic plants which may be mentioned are the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapes), with particular emphasis being given to maize, soya beans, wheat, rice, potatoes, cotton, sugar cane, tobacco and oilseed rape. Traits which are particularly emphasized are the increased resistance of the plants to insects, arachnids, nematodes and slugs and snails, as well as the increased resistance of the plants to one or more herbicides.

Commercially available examples of such plants, plant parts or plant seeds that may be treated with preference in accordance with the invention include commercial products, such as plant seeds, sold or distributed under the GENUITY®, DROUGHTGARD®, SMARTSTAX®, RIB COMPLETE®, ROUNDUP READY®, VT DOUBLE PRO®, VT TRIPLE PRO®, BOLLGARD II®, ROUNDUP READY 2 YIELD®, YIELDGARD®, ROUNDUP READY® 2 XTEND™, INTACTA RR2 PRO®, VISTIVE GOLD®, and/or XTENDFLEX™ trade names.

A TFL gene useful with this invention includes any TFL gene involved in the flowering process, which can confer a reduced or shortened time to flowering, a longer duration of flowering, a more determinate plant growth pattern and/or one or more improved yield characteristics (e.g., increased fruit production) in a Rosaceae plant or part thereof (e.g., a TFL1 protein/gene). In some embodiments, the TFL gene (a) comprises a nucleotide sequence having at least 80% % sequence identity (e.g., 80, 81, 82, 83, 84, 85, 86, 87, 88, 89, 90, 91, 92, 93, 94, 95, 96, 97, 98, 99, or 100%, optionally about 90, 91, 92, 93, 94, 95, 96, 97, 98, 99, or 100% or about 95, 96, 97, 98, 99, or 100% sequence identity) to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity (e.g., 80, 81, 82, 83, 84, 85, 86, 87, 88, 89, 90, 91, 92, 93, 94, 95, 96, 97, 98, 99, or 100%, optionally about 90, 91, 92, 93, 94, 95, 96, 97, 98, 99, or 100% or about 95, 96, 97, 98, 99, or 100% sequence identity) to any one of SEQ ID NOs:75-105, 109-113, 117-125, 129-139, 143-152, 155-161, 165-171, 175-183, 187-192, 217-233, 237-246, or 254-270, (c) encodes a sequence having at least 80% sequence identity (e.g., 80, 81, 82, 83, 84, 85, 86, 87, 88, 89, 90, 91, 92, 93, 94, 95, 96, 97, 98, 99, or 100%, optionally about 90, 91, 92, 93, 94, 95, 96, 97, 98, 99, or 100% or about 95, 96, 97, 98, 99, or 100% sequence identity) to any one of SEQ ID NOs:74, 108, 116, 128, 142, 154, 164, 174, 186, 216, 236 or 253, and/or (d) encodes a region having at least 80% sequence identity (e.g., 80, 81, 82, 83, 84, 85, 86, 87, 88, 89, 90, 91, 92, 93, 94, 95, 96, 97, 98, 99, or 100%, optionally about 90, 91, 92, 93, 94, 95, 96, 97, 98, 99, or 100% or about 95, 96, 97, 98, 99, or 100% sequence identity) to any one of the amino acid sequences of SEQ ID NOs:193-197, 247, 248, or 271-276.

Any mutation in a TFL gene that produces a non-functional TFL polypeptide may be used to produce Rosaceae plants or parts thereof of this invention having a reduced time to flowering, a longer duration of flowering, a more determinate plant growth pattern and/or one or more improved yield characteristics. In some embodiments, the mutation in the TFL gene may produce a TFL protein that is reduced in functionality (e.g., attenuated ability to function in its role in the flowering process) may be also used to produce Rosaceae plants or parts thereof of this invention having a reduced time to flowering, a longer duration of flowering, a more determinate plant growth pattern and/or one or more improved yield characteristics.

In some embodiments, the at least one mutation in an endogenous TFL gene is a null allele (e.g., produces a non-functional protein or no protein). In some embodiments, the at least one mutation in an endogenous TFL gene is a dominant negative mutation (e.g., produces a protein having aberrant function that interferes with the function wild type gene product). In some embodiments, the at least one mutation in an endogenous TFL gene in a Rosaceae plant may be a substitution, a deletion and/or an insertion. In some embodiments, the at least one mutation in an endogenous TFL gene in a Rosaceae plant may be a substitution, a deletion and/or an insertion that results in a null allele, semi-dominant allele, weak loss of function allele, a null allele, or a hypomorphic mutation and a Rosaceae plant exhibiting a phenotype of reduced time to flowering, a longer duration of flowering, a more determinate plant growth pattern and/or one or more improved yield characteristics. In some embodiments, the at least one mutation in an endogenous TFL gene in a Rosaceae plant may be a substitution, a deletion and/or an insertion that results in a dominant negative mutation, a semi-dominant mutation, a weak loss of function mutation, a null mutation, or a hypomorphic mutation and a Rosaceae plant exhibiting a phenotype of reduced time to flowering, a longer duration of flowering, a more determinate plant growth pattern and/or one or more improved yield characteristics. For example, the mutation may be a substitution, a deletion and/or an insertion of one or more amino acid residues (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more consecutive amino acid residues, or more amino acids of the transcription factor) or the mutation may be a substitution, a deletion and/or an insertion of at least 5 consecutive nucleotides (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more consecutive nucleotides (e.g., up to the full length of the TFL genomic sequence), or any range or value therein) (e.g., a base substitution, deletion and/or insertion) from the gene encoding the transcription factor. In some embodiments, the at least one mutation may be a base substitution to an A, a T, a G, or a C.

In some embodiments, a mutation in a TFL protein/gene produced by methods of this invention may be a deletion. In some embodiments, a deletion may result in a truncation of the TFL protein or a deletion of a portion or the entire TFL polypeptide. In some embodiments, the mutation may be an N-terminal truncation or a C-terminal truncation. In some embodiments, the deletion may be a within the polypeptide or may encompass the entire polypeptide. When the mutation results in a C-terminal truncation in the TFL protein, the C-terminal truncation may comprise a truncation of at least 1 amino acid residue (e.g., about 1, about 5, about 10, about 15, about 20, about 30, about 40 or about 50 amino acid residues to about 60, about 70, about 80, about 90, about 100, about 110, about 120, about 130, about 140, about 150, about 160, about 170, or about 172 consecutive amino acid residues or more) (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 171, 172 consecutive amino acid residues, or more, or any range or value therein) from the C- or N-terminus of the TFL protein (e.g., SEQ ID NOs:74, 108, 116, 128, 142, 154, 164, 174, 186, 216, 236 or 253). In some embodiments, the polynucleotide encoding a truncated TFL polypeptide may comprise a deletion of at least 3 consecutive base pairs (e.g., about 3, 4, 5, 6, 9, 12, 15, 18, 21, 24, 27, 30, 33, 36, 39, 40, 50, 100 consecutive base pairs to about 150, 200, 250, 300, 350, 400, 450, 500, 510, 515, 516, 517, 518, 519, 520, 525, 550, 600, 700, 800, 900, or more consecutive base pairs; e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350, 400, 450, 500, 505, 510, 515, 516, 517, 518, 519, 520, 525,550, 600, 650, 700, 750, 800, 850, 900, or 950 or more consecutive base pairs, or any range or value therein) from an endogenous gene encoding the TFL polypeptide (e.g., SEQ ID NOs:72, 73, 106, 107, 114, 115, 126, 127, 140, 141, 153, 162, 163, 172, 173, 184, 185, 214, 215, 234, 235, or 252). In some embodiments, a mutation in an endogenous TLF gene may result in a nucleic acid sequence having a at least 90% sequence identity to any one of SEQ ID NO:277-284, 295, 297, 298, 300, 302, 304, 306, 308, 309, 310, 311, or 313 and/or encode a mutated TFL1 polypeptide having at least 90% sequence identity to any one of SEQ ID NOs:285-291, 296, 299, 301, 303, 305, 307, or 312. In some embodiments, a mutated endogenous TFL gene may be a non-natural mutation.

A mutation in an endogenous gene encoding a TFL protein that provides Rosaceae plants with a phenotype of reduced time to flowering, a longer duration of flowering, a more determinate plant growth pattern and/or one or more improved yield characteristics may be a null allele. In some embodiments, a mutation in an endogenous gene encoding a TFL protein that provides Rosaceae plants that have a phenotype of reduced time to flowering, a longer duration of flowering, a more determinate plant growth pattern and/or one or more improved yield characteristics may be dominant negative mutation, a semi-dominant mutation, weak loss of function mutation, a null mutation, or a hypomorphic mutation, optionally where the mutation may be a non-natural mutation.

In some embodiments, a mutation in an endogenous TFL gene may be made following cleavage by an editing system that comprises a nuclease and a DNA-binding domain that binds to a target site within a target nucleic acid comprising a nucleotide sequence having at least 80% sequence identity to any one of the nucleotide sequence of SEQ ID NOs:72, 73, 106, 107, 114, 115, 126, 127, 140, 141, 153, 162, 163, 172, 173, 184, 185, 214, 215, 234, 235, or 252, or encoding a polypeptide comprising the sequence of any one of the amino acid sequences of SEQ ID NOs:74, 108, 116, 128, 142, 154, 164, 174, 186, 216, 236 or 253. In some embodiments, the nuclease cleaves the endogenous TFL gene, and a mutation is introduced into the endogenous TFL gene.

Further provided herein are guide nucleic acids (e.g., gRNA, gDNA, crRNA, crDNA) that bind to a target site in a TFL gene, wherein the endogenous TFL gene: (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276.

Additionally provided are guide nucleic acids that binds to a target site in a TFL gene, wherein the target site is in a region of the TFL gene having at least 80% sequence identity to any one of SEQ ID NOs:75-105, 109-113, 117-125, 129-139, 143-152, 155-161, 165-171, 175-183, 187-192, 217-233, 237-246, or 254-270. In some embodiment, a guide nucleic acid comprises a spacer having at least 80% sequence identity to the nucleotide sequence of any one of SEQ ID NOs:198-210, 211-213 or 249-251 optionally comprising the nucleotide sequence of any one of SEQ ID NOs:198-210, 211-213 or 249-251.

In some embodiments, a system is provided comprising a guide nucleic acid comprising a spacer sequence having the nucleotide sequence of any one of SEQ ID NOs:198-210, 211-213 or 249-251 and a CRISPR-Cas effector protein that associates with the guide nucleic acid. In some embodiments, the system may further comprise a tracr nucleic acid that associates with the guide nucleic acid and a CRISPR-Cas effector protein, optionally wherein the tracr nucleic acid and the guide nucleic acid are covalently linked.

As used herein, “a CRISPR-Cas effector protein in association with a guide nucleic acid” refers to the complex that is formed between a CRISPR-Cas effector protein and a guide nucleic acid in order to direct the CRISPR-Cas effector protein to a target site in a gene.

The invention further provides a gene editing system comprising a CRISPR-Cas effector protein in association with a guide nucleic acid and the guide nucleic acid comprises a spacer sequence that binds to an endogenous TFL gene, wherein the TFL gene: (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276. In some embodiments, a spacer sequence useful with this invention may bind to a target site comprising a sequence having at least 80% sequence identity to any one of the nucleotide sequences of SEQ ID NOs:72, 73, 106, 107, 114, 115, 126, 127, 140, 141, 153, 162, 163, 172, 173, 184, 185, 214, 215, 234, 235, or 252 or SEQ ID NOs:75-105, 109-113, 117-125, 129-139, 143-152, 155-161, 165-171, 175-183, 187-192, 217-233, 237-246, or 254-270, or a sequence having at least 80% sequence identity to a nucleotide sequence encoding any one of the amino acid sequences of SEQ ID NOs:198-210, 211-213 or 249-251. In some embodiments, a spacer sequence of the guide nucleic acid may comprise the nucleotide sequence of any one of SEQ ID NOs:198-213 or 249-251. In some embodiments, the gene editing system may further comprise a tracr nucleic acid that associates with the guide nucleic acid and a CRISPR-Cas effector protein, optionally wherein the tracr nucleic acid and the guide nucleic acid are covalently linked.

The present invention further provides a complex comprising a CRISPR-Cas effector protein comprising a cleavage domain and a guide nucleic acid (e.g., gRNA), wherein the guide nucleic acid binds to a target site in a TFL gene, wherein the TFL gene (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, wherein the cleavage domain cleaves a target strand in the TFL gene, wherein the cleavage domain cleaves a target strand in the TFL gene.

In some embodiments, expression cassettes are provided that comprise (a) a polynucleotide encoding CRISPR-Cas effector protein comprising a cleavage domain and (b) a guide nucleic acid that binds to a target site in an endogenous TFL gene, wherein the guide nucleic acid comprises a spacer sequence that is complementary to and binds: (i) a portion of nucleic acid having at least 80% sequence identity to any one of the nucleotide sequences of SEQ ID NOs:72, 73, 106, 107, 114, 115, 126, 127, 140, 141, 153, 162, 163, 172, 173, 184, 185, 214, 215, 234, 235, or 252; (ii) a portion of nucleic acid having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270; (iii) a portion of a nucleic acid encoding an amino acid sequence having at least 80% sequence identity to any one of the amino acid sequences SEQ ID NOs:74, 108, 116, 128, 142, 154, 164, 174, 186, 216, 236 or 253; and/or (iv) a portion of a nucleic acid encoding an amino acid sequence having at least 80% sequence identity to any one of the amino acid sequences of SEQ ID NOs:193-197, 247, 248, or 271-276.

Also provided herein is an endogenous TFL gene having a mutation, wherein the endogenous TFL gene having the mutation comprises a nucleic acid sequence having a mutation as described herein, optionally a mutation resulting in a truncated TFL polypeptide or no detectable TFL polypeptide. In some embodiments, a mutation in a TFL1 gene may be a non-natural mutation. Further provided is a nucleic acid encoding a null allele of a TFL gene, wherein the null allele when present in a Rosaceae plant results in a reduced time to flowering, a longer duration of flowering, a more determinate plant growth pattern and/or one or more improved yield characteristics in the Rosaceae plant. Additionally provided are nucleic acids encoding a dominant negative mutation of a TFL gene, which when present in a Rosaceae plant results in a reduced time to flowering, a longer duration of flowering, a more determinate plant growth pattern and/or one or more improved yield characteristics in the Rosaceae plant. Also provided herein, are nucleic acids encoding a semi-dominant mutation, a weak loss of function mutation, a null mutation, or a hypomorphic mutation, wherein the semi-dominant mutation, weak loss of function mutation, null mutation, or hypomorphic mutation of a TFL gene as described herein, which when present in a Rosaceae plant results in a reduced time to flowering in the Rosaceae plant and/or a Rosaceae plant having a longer duration of flowering, a more determinate plant growth pattern and/or one or more improved yield characteristics compared to a control Rosaceae plant. In some embodiments, a mutation in an endogenous TLF gene may result in a nucleic acid sequence having a at least 90% sequence identity to any one of SEQ ID NO:277-284, 295, 297, 298, 300, 302, 304, 306, 308, 309, 310, 311, or 313 and/or encode a mutated TFL1 polypeptide having at least 90% sequence identity to any one of SEQ ID NOs:285-291, 296, 299, 301, 303, 305, 307, or 312. In some embodiments, the mutated endogenous TFL gene comprises a non-natural mutation.

Nucleic acid constructs of the invention (e.g., a construct comprising a sequence specific DNA binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids (e.g., endogenous TFL genes of Rosaceae plants) and/or their expression.

Any Rosaceae plant (e.g., Prunus spp., Rubus spp., Fragaria spp., Malus spp.) comprising an endogenous TFL gene that is involved in regulation of flowering time in a Rosaceae plant may be modified (e.g., mutated, e.g., base edited, cleaved, nicked, etc.) as described herein (e.g., using the polypeptides, polynucleotides, RNPs, nucleic acid constructs, expression cassettes, and/or vectors of the invention) to reduce or shorten the time to flowering, longer duration of flowering, more determinate plant growth pattern and/or one or more improved yield characteristics in the plant as compared to the unmodified Rosaceae plant. For example, the invention may provide a Rosaceae plant having a reduced time to flowering (earlier flowering) of about 5%, 6%, 7%, 8%, 9%, or 10% to about 95%, 96%, 97%, 98%, 99% or 100% (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% or any range or value therein) as compared to a Rosaceae plant that is devoid of the mutated endogenous TFL gene.

In some embodiments, a Rosaceae plant is provide having increased duration of flowering (lengthened time of flowering) by about 30% to about 170%, (e.g., about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 120, 125, 130, 135, 140, 145, 150, 155, 160, 161, 162, 163, 164, 165, 166, 167, 168, 169, or 170% or any range or value) as compared to a Rosaceae plant that is devoid of the mutated endogenous TFL gene.

In some embodiments, a Rosaceae plant produced as described herein starts flowering earlier and continues to flower until it dies (e.g., killed by a frost or freeze), e.g., continuous flowering. Thus, the Rosaceae plant exhibits a reduced time to flowering and an increased duration of flowering as compared to a Rosaceae plant devoid of the mutation.

In some embodiments, the invention may provide a Rosaceae plant having a more determinate plant growth pattern (e.g., having stems that are about 30% to about 85% shorter than a control plant (e.g., having stems about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85% shorter) as compared to a Rosaceae plant that is devoid of the mutated endogenous TFL gene.

In some embodiments, the invention may provide a Rosaceae plant having one or more improved yield characteristics (e.g., 1, 2, 3, 4, 5, 6, 7, 8, 9, 10 or more) as compared to a Rosaceae plant that is devoid of the mutated endogenous TFL gene. In some embodiments, an improved yield characteristic may be an increase in marketable fruit yield, optionally an increase of about 50% to about 750% of marketable fruit yield (e.g., about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 130, 140, 150, 160, 170, 180, 190, 200, 225, 250, 275, 300, 325, 350, 375, 400, 425, 450, 475, 500, 525, 550, 575, 600, 625, 650, 675, 700, 725, or 750% increase in marketable fruit weight) as compared to a control plant (e.g., devoid of the modification in a TFL1 gene as described herein. “Marketable fruit” includes fruit having a ripe appearance and is generally free of disease or rot. In the case of berries, “marketable fruit” generally includes berries having a weight range of 6 g to 10 g per berry. In some embodiments, the invention may provide a Rosaceae plant having any combination of a reduced time to flowering, a longer duration of flowering, a more determinate plant growth pattern and/or one or more improved yield characteristics.

A Rosaceae plant and/or plant part that may be modified as described herein may be any Rosaceae genus, species, variety and/or cultivar. Non-limiting examples of Rosaceae plants that may be modified as described herein include, but are not limited to, Rubus spp. (e.g., blackberry, black raspberry or raspberry, and the like), Prunus spp., Frageria spp., and/or Malus spp. Example Rubus plants useful with the invention can include, but are not limited to, Rubus occidentalis L., Rubus pergratus Blanch., Rubus oklahomus L. H. Bailey Rubus originalis L. H. Bailey, Rubus ortivus (L. H. Bailey) L. H. Bailey, Rubus parcifrondifer L. H. Bailey, Rubus odoratus L., Rubus parvifolius L., Rubus pedatus Sm., and Rubus phoenicolasius Maxim. Example Prunus spp. plants useful with the invention can include, but are not limited to, P. persica, P. pyrifolia, P. serotina, P. armeniaca, P. spinosa, P. avium, or P. dulcis (e.g., plum, apricot, cherry, nectarine, peach, almond, chokecherry, cherry laurel, and blackthorn). Example Fragaria spp. plants useful with the invention can include, but are not limited to, F. vesca, Fragaria x ananassa Duchesne, or F. chiloensis. Example Malus spp. plants useful with the invention can include, but are not limited to, M. domesticus, Pyrus communis, Cydonia oblonga, Crataegus spp., Chaenomeles spp., or Amelanchier spp. In some embodiments, a Rosaceae plant or part thereof useful with this invention is a caneberry or stone fruit, optionally a blackberry, a black raspberry, a cherry, a plum or a peach.

The term “plant part,” as used herein, includes but is not limited to reproductive tissues (e.g., petals, sepals, stamens, pistils, receptacles, anthers, pollen, flowers, fruits, flower bud, ovules, seeds, and embryos); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus. The term “stem” as used herein refers the above ground structural axis of the plant consisting of both nodes (e.g., leaves and flowers) and internodes (e.g., connecting material between nodes).

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like. In some aspects of the invention, the plant part can be a plant germplasm. In some aspects, a plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In some embodiments of the invention, a transgenic tissue culture or transgenic plant cell culture is provided, wherein the transgenic tissue or cell culture comprises a nucleic acid molecule/nucleotide sequence of the invention. In some embodiments, transgenes may be eliminated from a plant developed from the transgenic tissue or cell by breeding of the transgenic plant with a non-transgenic plant and selecting among the progeny for the plants comprising the desired gene edit and not the transgenes used in producing the edit.

An editing system useful with this invention can be any site-specific (sequence-specific) genome editing system now known or later developed, which system can introduce mutations in a target specific manner. For example, an editing system (e.g., site- or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which can comprise one or more polypeptides and/or one or more polynucleotides that when expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide.

In some embodiments, an editing system can comprise one or more sequence-specific nucleic acid binding domains (DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN). In some embodiments, an editing system can comprise one or more polypeptides that include, but are not limited to, a deaminase (e.g., a cytosine deaminase, an adenine deaminase), a reverse transcriptase, a Dna2 polypeptide, and/or a 5′ flap endonuclease (FEN). In some embodiments, an editing system can comprise one or more polynucleotides, including, but is not limited to, a CRISPR array (CRISPR guide) nucleic acid, extended guide nucleic acid, and/or a reverse transcriptase template.

In some embodiments, a method of modifying or editing a TFL gene may comprise contacting a target nucleic acid (e.g., a nucleic acid encoding a TFL protein) with a base-editing fusion protein (e.g., a sequence specific DNA binding protein (e.g., a CRISPR-Cas effector protein or domain) fused to a deaminase domain (e.g., an adenine deaminase and/or a cytosine deaminase) and a guide nucleic acid, wherein the guide nucleic acid is capable of guiding/targeting the base editing fusion protein to the target nucleic acid, thereby editing a locus within the target nucleic acid. In some embodiments, a base editing fusion protein and guide nucleic acid may be comprised in one or more expression cassettes. In some embodiments, the target nucleic acid may be contacted with a base editing fusion protein and an expression cassette comprising a guide nucleic acid. In some embodiments, the sequence-specific nucleic acid binding fusion proteins and guides may be provided as ribonucleoproteins (RNPs). In some embodiments, a cell may be contacted with more than one base-editing fusion protein and/or one or more guide nucleic acids that may target one or more target nucleic acids in the cell.

In some embodiments, a method of modifying or editing a TFL gene may comprise contacting a target nucleic acid (e.g., a nucleic acid encoding a TFL protein) with a sequence-specific nucleic acid binding fusion protein (e.g., a sequence-specific DNA binding protein (e.g., a CRISPR-Cas effector protein or domain) fused to a peptide tag, a deaminase fusion protein comprising a deaminase domain (e.g., an adenine deaminase and/or a cytosine deaminase) fused to an affinity polypeptide that is capable of binding to the peptide tag, and a guide nucleic acid, wherein the guide nucleic acid is capable of guiding/targeting the sequence-specific nucleic acid binding fusion protein to the target nucleic acid and the sequence-specific nucleic acid binding fusion protein is capable of recruiting the deaminase fusion protein to the target nucleic acid via the peptide tag-affinity polypeptide interaction, thereby editing a locus within the target nucleic acid. In some embodiments, the sequence-specific nucleic acid binding fusion protein may be fused to the affinity polypeptide that binds the peptide tag and the deaminase may be fused to the peptide tag, thereby recruiting the deaminase to the sequence-specific nucleic acid binding fusion protein and to the target nucleic acid. In some embodiments, the sequence-specific binding fusion protein, deaminase fusion protein, and guide nucleic acid may be comprised in one or more expression cassettes. In some embodiments, the target nucleic acid may be contacted with a sequence-specific binding fusion protein, deaminase fusion protein, and an expression cassette comprising a guide nucleic acid. In some embodiments, the sequence-specific nucleic acid binding fusion proteins, deaminase fusion proteins and guides may be provided as ribonucleoproteins (RNPs).

In some embodiments, methods such as prime editing may be used to generate a mutation in an endogenous TFL gene. In prime editing, RNA-dependent DNA polymerase (reverse transcriptase, RT) and reverse transcriptase templates (RT template) are used in combination with sequence specific nucleic acid binding domains that confer the ability to recognize and bind the target in a sequence-specific manner, and which can also cause a nick of the PAM-containing strand within the target. The nucleic acid binding domain may be a CRISPR-Cas effector protein and in this case, the CRISPR array or guide RNA may be an extended guide that comprises an extended portion comprising a primer binding site (PSB) and the edit to be incorporated into the genome (the template). Similar to base editing, prime editing can take advantages of the various methods of recruiting proteins for use in the editing to the target site, such methods including both non-covalent and covalent interactions between the proteins and nucleic acids used in the selected process of genome editing.

As used herein, a “CRISPR-Cas effector protein” is a protein or polypeptide or domain thereof that cleaves or cuts a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid.

In some embodiments, a sequence-specific DNA binding domain may be a CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas12 effector protein.

In some embodiments, a CRISPR-Cas effector protein may include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g., Cas9 nickase, Cas12a nickase.

A CRISPR Cas9 effector protein or CRISPR Cas9 effector domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophilus), Lactobacillus spp., Bifzdobacterium spp., Kandleria spp., Leuconostoc spp., Oenococcus spp., Pediococcus spp., Weissella spp., and/or Olsenella spp. Example Cas9 sequences include, but are not limited to, the amino acid sequences of SEQ ID NO:59 and SEQ ID NO:60 or the nucleotide sequences of any one of SEQ ID NOs:61-71.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339(6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 polypeptide derived from Streptococcus mutans and recognizes the PAM sequence motif NGG and/or NAAR (R=A or G) (See, e.g., Deveau et al, J BACTERIOL 2008; 190(4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif N GRRT (R=A or G). In some embodiments, the CRISPR-Cas effector protein may be a Cas9 polypeptide derived from S. aureus, which recognizes the PAM sequence motif N GRRV (R=A or G). In some embodiments, the CRISPR-Cas effector protein may be a Cas9 polypeptide that is derived from Neisseria meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

In some embodiments, the CRISPR-Cas effector protein may be derived from Cas12a, which is a Type V Clustered Regularly Interspaced Short Palindromic Repeats (CRISPR)-Cas nuclease see, e.g., SEQ ID NOs:1-20). Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity.

Any deaminase domain/polypeptide useful for base editing may be used with this invention. In some embodiments, the deaminase domain may be a cytosine deaminase domain or an adenine deaminase domain. A cytosine deaminase (or cytidine deaminase) useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally occurring cytosine deaminase, including but not limited to a primate (e.g., a human, monkey, chimpanzee, gorilla), a dog, a cow, a rat, or a mouse. Thus, in some embodiments, an cytosine deaminase useful with the invention may be about 70% to about 100% identical to a wild type cytosine deaminase (e.g., about 70%, 71%, 72%, 73%, 74%, 75%, 76%, 77%, 78%, 79%, 80%, 81%, 82%, 83%, 84%, 85%8, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an at CDA1 (e.g., At2g19570), and evolved versions of the same (e.g., SEQ ID NO:27, SEQ ID NO:28 or SEQ ID NO:29). In some embodiments, the cytosine deaminase may be an APOBEC1 deaminase having the amino acid sequence of SEQ ID NO:23. In some embodiments, the cytosine deaminase may be an APOBEC3A deaminase having the amino acid sequence of SEQ ID NO:24. In some embodiments, the cytosine deaminase may be an CDA1 deaminase, optionally a CDA1 having the amino acid sequence of SEQ ID NO:25. In some embodiments, the cytosine deaminase may be a FERNY deaminase, optionally a FERNY having the amino acid sequence of SEQ ID NO:26. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., an evolved deaminase). In some embodiments, a cytosine deaminase useful with the invention may be about 70% to about 99.5% identical (e.g., about 70%, 71%, 72%, 73%, 74%, 75%, 76%, 77%, 78%7, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of SEQ ID NO:23, SEQ ID NO:24, SEQ ID NO:25 or SEQ ID NO:26 (e.g., at least 80%, at least 85%, at least 90%, at least 92%, at least 95%, at least 96%, at least 97%, at least 98%, at least 99%, or at least 99.5% identical to the amino acid sequence of SEQ ID NO:23, SEQ ID NO:24, SEQ ID NO:25, SEQ ID NO:26, SEQ ID NO:27, SEQ ID NO:28 or SEQ ID NO:29). In some embodiments, a polynucleotide encoding a cytosine deaminase may be codon optimized for expression in a plant and the codon optimized polypeptide may be about 70% to 99.5% identical to the reference polynucleotide.

In some embodiments, a nucleic acid construct of this invention may further encode a uracil glycosylase inhibitor (UGI) (e.g., uracil-DNA glycosylase inhibitor) polypeptide/domain. Thus, in some embodiments, a nucleic acid construct encoding a CRISPR-Cas effector protein and a cytosine deaminase domain (e.g., encoding a fusion protein comprising a CRISPR-Cas effector protein domain fused to a cytosine deaminase domain, and/or a CRISPR-Cas effector protein domain fused to a peptide tag or to an affinity polypeptide capable of binding a peptide tag and/or a deaminase protein domain fused to a peptide tag or to an affinity polypeptide capable of binding a peptide tag) may further encode a uracil-DNA glycosylase inhibitor (UGI), optionally wherein the UGI may be codon optimized for expression in a plant. In some embodiments, the invention provides fusion proteins comprising a CRISPR-Cas effector polypeptide, a deaminase domain, and a UGI and/or one or more polynucleotides encoding the same, optionally wherein the one or more polynucleotides may be codon optimized for expression in a plant. In some embodiments, the invention provides fusion proteins, wherein a CRISPR-Cas effector polypeptide, a deaminase domain, and a UGI may be fused to any combination of peptide tags and affinity polypeptides as described herein, thereby recruiting the deaminase domain and UGI to the CRISPR-Cas effector polypeptide and a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

A “uracil glycosylase inhibitor” useful with the invention may be any protein that is capable of inhibiting a uracil-DNA glycosylase base-excision repair enzyme. In some embodiments, a UGI domain comprises a wild type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:41 or a polypeptide having about 70% to about 99.5% sequence identity to the amino acid sequence of SEQ ID NO:41 (e.g., at least 80%, at least 85%, at least 90%, at least 92%, at least 95%, at least 96%, at least 97%, at least 98%, at least 99%, or at least 99.5% identical to the amino acid sequence of SEQ ID NO:41). For example, in some embodiments, a UGI domain may comprise a fragment of the amino acid sequence of SEQ ID NO:41 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:41. In some embodiments, a UGI domain may be a variant of a known UGI (e.g., SEQ ID NO:41) having about 70% to about 99.5% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%0, 90%, 91%, 92%, 93%, 94%, 95%, 96%, 97%, 98%, 99%, 99.5% sequence identity, and any range or value therein) to the known UGI. In some embodiments, a polynucleotide encoding a UGI may be codon optimized for expression in a plant (e.g., a plant) and the codon optimized polypeptide may be about 70% to about 99.5% identical to the reference polynucleotide.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild type E. coli TadA comprises the amino acid sequence of SEQ ID NO:30. In some embodiments, a mutated/evolved E. coli TadA* comprises the amino acid sequence of SEQ ID NOs:31-40 (e.g., SEQ ID NOs:31, 32, 33, 34, 35, 36, 37, 38, 39 or 40). In some embodiments, a polynucleotide encoding a TadA/TadA* may be codon optimized for expression in a plant.

A cytosine deaminase catalyzes cytosine deamination and results in a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “−”, complementary) strand of the target nucleic acid.

The nucleic acid constructs of the invention encoding a base editor comprising a sequence-specific DNA binding protein and a cytosine deaminase polypeptide, and nucleic acid constructs/expression cassettes/vectors encoding the same, may be used in combination with guide nucleic acids for modifying target nucleic acid including, but not limited to, generation of C→T or G→A mutations in a target nucleic acid including, but not limited to, a plasmid sequence; generation of C→T or G→A mutations in a coding sequence to alter an amino acid identity; generation of C→T or G→A mutations in a coding sequence to generate a stop codon; generation of C→T or G→A mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention encoding a base editor comprising a sequence-specific DNA binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention comprising a CRISPR-Cas effector protein or a fusion protein thereof may be used in combination with a guide RNA (gRNA, CRISPR array, CRISPR RNA, crRNA), designed to function with the encoded CRISPR-Cas effector protein or domain, to modify a target nucleic acid. A guide nucleic acid useful with this invention comprises at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex (e.g., a CRISPR-Cas effector fusion protein (e.g., CRISPR-Cas effector domain fused to a deaminase domain and/or a CRISPR-Cas effector domain fused to a peptide tag or an affinity polypeptide to recruit a deaminase domain and optionally, a UGI) to the target nucleic acid, wherein the target nucleic acid may be modified (e.g., cleaved or edited) or modulated (e.g., modulating transcription) by the deaminase domain.

As an example, a nucleic acid construct encoding a Cas9 domain linked to a cytosine deaminase domain (e.g., fusion protein) may be used in combination with a Cas9 guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas9 domain linked to an adenine deaminase domain (e.g., fusion protein) may be used in combination with a Cas9 guide nucleic acid to modify a target nucleic acid, wherein the adenine deaminase domain of the fusion protein deaminates an adenosine base in the target nucleic acid, thereby editing the target nucleic acid.

Likewise, a nucleic acid construct encoding a Cas12a domain (or other selected CRISPR-Cas nuclease, e.g., C2c1, C2c3, Cas12b, Cas12c, Cas12d, Cas12e, Cas13a, Cas13b, Cas13c, Cas13d, Cas1, CaslB, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5) linked to a cytosine deaminase domain or adenine deaminase domain (e.g., fusion protein) may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof, a repeat of a Type V C2c1 CRISPR Cas system, or a fragment thereof, a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Cas1, CaslB, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35(Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more (e.g., 99.1, 99.2, 99.3, 99.4, 99.5, 99.6, 99.7, 99.8, 99.9, or 100%)) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer) (e.g., a portion of consecutive nucleotides of a sequence (a) having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, or SEQ ID NOs:75-105, 109-113, 117-125, 129-139, 143-152, 155-161, 165-171, 175-183, 187-192, 217-233, 237-246, or 254-270; and/or (b) encoding an amino acid sequence having at least 80% sequence identity to any one of SEQ ID NOs:74, 108, 116, 128, 142, 154, 164, 174, 186, 216, 236 or 253, or SEQ ID NOs:193-197, 247, 248, or 271-276). In some embodiments, a spacer sequence (e.g., one or more spacers) may include, but is not limited to, the nucleotide sequences of any one of SEQ ID NOs:193-197, 247, 248, or 271-276, or any combination thereof. The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more (e.g., 99.1, 99.2, 99.3, 99.4, 99.5, 99.6, 99.7, 99.8, 99.9, or 100%)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be identical to a target DNA, while the 3′ region of the spacer may be substantially complementary to the target DNA (e.g., Type V CRISPR-Cas), or the 3′ region of a spacer sequence of a guide nucleic acid may be identical to a target DNA, while the 5′ region of the spacer may be substantially complementary to the target DNA (e.g., Type II CRISPR-Cas), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of a plant's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

| | RNA Spacer |
|---|---|
| (SEQ ID NO: 42) | |
| | 5′-NNNNNNNNNNNNNNNNNNN-3′ |
| | \|\|\|\|\|\|\|\|\|\|\|\|\|\|\|\|\|\|\| |
| | Target strand |
| (SEQ ID NO: 43) | |
| | 3′AAANNNNNNNNNNNNNNNNNNN-5′ |
| | \|\|\|\| |
| | Non-target strand |
| (SEQ ID NO: 44) | |
| | 5′TTTNNNNNNNNNNNNNNNNNNN-3′ |

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encoding a base editor (e.g., a construct comprising a CRISPR-Cas effector protein and a deaminase domain (e.g., a fusion protein)) or the components for base editing (e.g., a CRISPR-Cas effector protein fused to a peptide tag or an affinity polypeptide, a deaminase domain fused to a peptide tag or an affinity polypeptide, and/or a UGI fused to a peptide tag or an affinity polypeptide), may be comprised on the same or on a separate expression cassette or vector from that comprising the one or more guide nucleic acids. When the nucleic acid construct encoding a base editor or the components for base editing is/are comprised on separate expression cassette(s) or vector(s) from that comprising the guide nucleic acid, a target nucleic acid may be contacted with (e.g., provided with) the expression cassette(s) or vector(s) encoding the base editor or components for base editing in any order from one another and the guide nucleic acid, e.g., prior to, concurrently with, or after the expression cassette comprising the guide nucleic acid is provided (e.g., contacted with the target nucleic acid).

Fusion proteins of the invention may comprise sequence-specific nucleic acid binding domains (e.g., sequence-specific DNA binding domains), CRISPR-Cas polypeptides, and/or deaminase domains fused to peptide tags or affinity polypeptides that interact with the peptide tags, as known in the art, for use in recruiting the deaminase to the target nucleic acid. Methods of recruiting may also comprise guide nucleic acids linked to RNA recruiting motifs and deaminases fused to affinity polypeptides capable of interacting with RNA recruiting motifs, thereby recruiting the deaminase to the target nucleic acid. Alternatively, chemical interactions may be used to recruit polypeptides (e.g., deaminases) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22(4):413-420 (2013)), U.S. Pat. No. 9,982,053, each of which are incorporated by reference in their entireties for the teachings relevant to affibodies, anticalins, monobodies and/or DARPins. Example peptide tag sequences and their affinity polypeptides include, but are not limited to, the amino acid sequences of SEQ ID NOs:45-47.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases). Example RNA recruiting motifs and their affinity polypeptides include, but are not limited to, the sequences of SEQ ID NOs:48-58.

In some embodiments, a polypeptide fused to an affinity polypeptide may be a reverse transcriptase and the guide nucleic acid may be an extended guide nucleic acid linked to an RNA recruiting motif. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion.

In some embodiments of the invention, an extended guide RNA and/or guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF).

In some embodiments, the components for recruiting polypeptides and nucleic acids may those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together, e.g., dihyrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant (e.g., a Rosaceae plant).

Further provided herein are cells comprising one or more polynucleotides, guide nucleic acids, nucleic acid constructs, expression cassettes or vectors of the invention.

The nucleic acid constructs of the invention (e.g., a construct comprising a sequence specific DNA binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids and/or their expression.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1. Editing Strategies

A strategy to generate knock-out or knock-down edits in the blackberry (Rubus subsp. rubus) TFL genes SEQ ID NO:214 for proprietary blackberry line A and SEQ ID NO:234 for proprietary blackberry line B and SEQ ID NO:252 for proprietary blackberry line C was developed to alter the time to initiate flowering. All of the blackberry lines are polyploid and contain two TFL gene loci for a total of four copies of the TFL gene in each line. To generate a range of alleles, multiple CRISPR guide nucleic acids comprising spacers (SEQ ID NOs:249-251 (see Table 1)) having complementarity to targets within the TFL genes were designed and placed into multiple constructs.

Lines carrying edits in the TFL genes were screened and those that showed about 10% of the sequencing reads having edits in the targeted gene were advanced to the next generation.

TABLE 1
spacers and plant lines containing the targeted TFL genes

| Spacer name | Plant line |
|---|---|
| PWsp265 (SEQ ID | Blackberry line A (TFL genomic sequence SEQ ID |
| NO: 249) | NO: 214) |
| PWsp266 (SEQ ID | Blackberry line A (TFL genomic sequence SEQ ID |
| NO: 250) | NO: 214) |
| PWsp592 (SEQ ID | Blackberry line B TFL genomic sequence SEQ ID |
| NO: 251) | NO: 234) |

### Example 2. Edited TFL Alleles of Blackberry Line A

A blackberry line A plant was identified which contained edits in each of the four alleles of the two TFL genes. Allele 1 in the edited line contained a compound deletion in exon 1 in which 9 bp were deleted followed by a second deletion of 6 bp. The deletions in allele 1 are both in-frame deletions. Allele 2 in the edited line contained a compound deletion in exon 1 in which 16 bp were deleted followed by a second deletion of 4 bp. The deletions in allele 2 create an out of frame mutation, which results in a premature stop codon. Allele 3 in the edited line contained an 87 bp deletion in exon 1, which created an in-frame mutation. Allele 4 in the edited line contained a compound deletion in exon 1 in which 16 bp were deleted followed by a 4 bp deletion. The deletions in allele 4 create an out-of-frame mutation which results in a premature stop codon.

TABLE 2
Blackberry line A TFL alleles

| Allele | | SEQ ID |
|---|---|---|
| number | Edits | NO: |
| 1 | CTTGATTCC 9 bp deletion | |
| 1 | CTCTTA 6 bp deletion | |
| 2 | AGATGTTCTTGATTCC 16 bp deletion | 292 |
| 2 | CTTA 4 bp deletion | |
| 3 | AGATGTTCTTGATTCCTTCACCCCAACTACCAAAATGAT | 293 |
| | TGTCACTTACACTACGAGGCACGTCTCTAATGGATATGA | |
| | GCTCTTACC 87 bp deletion | |
| 4 | AGATGTTCTTGATTCC 16 bp deletion | 294 |
| 4 | CTCTTAC 7 bp deletion | |

### Example 3. Edited TFL Alleles Blackberry Line C Variety

TFL edited blackberry plants of primocane-flowering genotype blackberry line C were recovered from a transformation process (E0s) as described in Example 1 using the spacers PWsp265 (SEQ ID NO:249) and PWsp266 (SEQ ID NO:250). The TFL edits were confirmed by molecular screening and six edited events were identified for further analysis. In the six edited events, >95% of the sequencing reads supported the presence of alleles of TFL, which were distinct from the wild type sequence, indicating all four copies of the TFL gene were edited. All edits occurred within exon 1 of the TFL gene, and included small in-frame deletions, large in-frame deletion, or small or large deletions resulting in a change in the reading frame, which caused an early stop codon as set forth in Table 3.

It is noted that the genomic sequence for CE145602 Allele 3 and CE1270701 Allele 3 were the same sequence. Additionally, the genomic sequence for CE125667 Allele 1 and CE120701 Allele 2 were also the same sequence. All of the edited alleles of TFL1 in blackberry variety C were generated with the use of the same spacers PWsp265 and PWsp266 and the edited alleles noted above were independently generated in the referenced plants.

Due to the polyploid nature of the blackberry varieties used in this example, it is not yet possible to distinctly separate all four the TFL1 alleles in the wild type plant. The sequence generated clearly identifies the four TFL1 alleles, but does not allow for resolution of each of the alleles independently. As such, the sequence for the TFL1 genes in any particular blackberry variety is a consensus sequence of all four TFL1 alleles in that particular variety.

TABLE 3
Edited alleles

| TFL edited | | |
|---|---|---|
| allele | Edit description | notes |
| CE145602 | deletion of 20 bp starting at | Out of frame mutation |
| Allele 1 | position 2353 of SEQ ID | resulting in an early stop |
| (SEQ ID | NO: 252; and 5 bp starting at | codon (SEQ ID NO: 285) |
| NO: 277) | position 2435 of SEQ ID | |
| | NO: 252 | |
| CE145602 | deletion of 15 bp starting at | Out of frame mutation |
| Allele 2 | position 2355 of SEQ ID | resulting in an early stop |
| (SEQ ID | NO: 252; and 5 bp starting at | codon (SEQ ID NO: 286) |
| NO: 278) | position 2435 of SEQ ID | |
| | NO: 252 | |
| CE145602 | deletion of 9 bp starting at | Out-of-frame mutation |
| Allele 3 | position 2361 of SEQ ID | resulting in an early stop |
| (SEQ ID | NO: 252; and 8 bp starting at | codon (SEQ ID NO: 287) |
| NO: 279) | position 2434 of SEQ ID | |
| | NO: 252 | |
| CE145602 | Deletion of 79 bp starting at | Out-of-frame mutation |
| Allele 4 | position 2361 of SEQ ID | resulting in an early stop |
| (SEQ ID | NO: 252 | codon (SEQ ID NO: 288) |
| NO: 280) | | |
| CE119211 | deletion of 16 bp starting at | Out of frame mutation |
| Allele 1 | position 2354 of SEQ ID | resulting in an early stop |
| (SEQ ID | NO: 252; and 7 bp deletion | codon (SEQ ID NO: 289) |
| NO: 281) | starting at position 2433 of | |
| | SEQ ID NO: 252 | |
| CE119211 | deletion of 16 bp starting at | Out-of-frame mutation |
| Allele 2 | position 2354 of SEQ ID | resulting in an early stop |
| (SEQ ID | NO: 252; and 5 bp deletion | codon (SEQ ID NO: 289) |
| NO: 282) | starting at position 2435 of | |
| | SEQ ID NO: 252 | |
| CE119211 | Deletion of 87 bp starting at | Out-of-frame mutation |
| Allele 3 | position 2354 of SEQ ID | resulting in an early stop |
| (SEQ ID | NO: 252 | codon (SEQ ID NO: 290) |
| NO: 283) | | |
| CE119211 | deletion of 9 bp starting at | In-frame deletion giving |
| Allele 4 | position 2361 of SEQ ID | rise to the amino acid |
| (SEQ ID | NO: 252; and 6 bp starting at | sequence SEQ ID NO: 291 |
| NO: 284) | position 2433 of SEQ ID | |
| | NO: 252 | |
| CE120701 | Deletion of 85 bp starting at | Out-of-frame mutation |
| Allele 1 | position 2359 of SEQ ID | resulting in an early stop |
| (SEQ ID | NO: 252 | codon (SEQ ID NO: 296) |
| NO: 295) | | |
| CE120701 | Deletion of 79 bp starting at | Out-of-frame mutation |
| Allele 2 | position 2361 of SEQ ID | resulting in an early stop |
| (SEQ ID | NO: 252 | codon (SEQ ID NO: 288) |
| NO: 297) | | |
| CE120701 | deletion of 6 bp starting at | In-frame mutation resulting |
| Allele 3 | position 2363 of SEQ ID | in amino acid sequence of |
| (SEQ ID | NO: 252; and 6 bp starting at | SEQ ID NO: 299 |
| NO: 298) | position 2433 of SEQ ID | |
| | NO: 252 | |
| CE120701 | Edit not resolved | Edit not resolved |
| Allele 4 | | |
| CE120713 | deletion of 15 bp starting at | In-frame mutation resulting |
| Allele 1 | position 2355 of SEQ ID | in amino acid sequence of |
| (SEQ ID | NO: 252; and 9 bp starting at | SEQ ID NO: 301 |
| NO: 300) | position 2433 of SEQ ID | |
| | NO: 252 | |
| CE120713 | deletion of 13 bp starting at | Out-of-frame mutation |
| Allele 2 | position 2356 of SEQ ID | resulting in an early stop |
| (SEQ ID | NO: 252; and 5 bp starting at | codon (SEQ ID NO: 303) |
| NO: 302) | position 2435 of SEQ ID | |
| | NO: 252 | |
| CE120713 | deletion of 11 bp starting at | Out-of-frame mutation |
| Allele 3 | position 2359 of SEQ ID | resulting in an early stop |
| (SEQ ID | NO: 252; and 5 bp starting at | codon (SEQ ID NO: 305) |
| NO: 304) | position 2435 of SEQ ID | |
| | NO: 252 | |
| CE120713 | Deletion of 77 bp starting at | Out-of-frame mutation |
| Allele 4 | position 2362 of SEQ ID | resulting in an early stop |
| (SEQ ID | NO: 252 | codon (SEQ ID NO: 307) |
| NO: 306) | | |
| CE145602 | deletion of 20 bp starting at | Out-of-frame mutation |
| Allele 1 | position 2353 of SEQ ID | resulting in an early stop |
| (SEQ ID | NO: 252; and 5 bp starting at | codon (SEQ ID NO: 285) |
| NO: 308) | position 2435 of SEQ ID | |
| | NO: 252 | |
| CE145602 | deletion of 15 bp starting at | Out-of-frame mutation |
| Allele 2 | position 2355 of SEQ ID | resulting in an early stop |
| (SEQ ID | NO: 252; and 5 bp starting at | codon (SEQ ID NO: 286) |
| NO: 309) | position 2435 of SEQ ID | |
| | NO: 252 | |
| CE145602 | Deletion of 79 bp starting at | Out-of-frame mutation |
| Allele 3 | position 2361 of SEQ ID | resulting in an early stop |
| (SEQ ID | NO: 252 | codon (SEQ ID NO: 288) |
| NO: 298) | | |
| CE145602 | Deletion of 9 bp deletion | Out-of-frame mutation |
| Allele 4 | starting at position 2361 of | resulting in an early stop |
| (SEQ ID | SEQ ID NO: 252; and 8 bp | codon (SEQ ID NO: 287) |
| NO: 310) | deletion starting at position | |
| | 2434 of SEQ ID NO: 252 | |
| CE125667 | Deletion of 79 bp starting at | Out-of-frame mutation |
| Allele 1 | position 2361 of SEQ ID | resulting in an early stop |
| (SEQ ID | NO: 252 | codon (SEQ ID NO: 288) |
| NO: 297) | | |
| CE125667 | deletion of 8 bp starting at | Out-of-frame mutation |
| Allele 2 | position 2361 of SEQ ID | resulting in an early stop |
| (SEQ ID | NO: 252; and 7 bp starting at | codon (SEQ ID NO: 312) |
| NO: 311) | position 2433 of SEQ ID | |
| | NO: 252 | |
| CE125667 | deletion of 8 bp starting at | Out-of-frame mutation |
| Allele 3 | position 2361 of SEQ ID | resulting in an early stop |
| (SEQ ID | NO: 252; and 7 bp starting at | codon (SEQ ID NO: 312) |
| NO: 313) | position 2436 of SEQ ID | |
| | NO: 252 | |
| CE125667 | Edit not resolved | Edit not resolved |
| Allele 4 | | |

### Example 4. Phenotype Analysis of Flowering Time of BK13 TFL Edited Lines

The six edited BK13 lines described in Example 3 were evaluated under greenhouse conditions. The six edited lines were compared to a control group of three unedited plants of the same genotype (i.e., BK13), which were recovered from the same transformation process. Plants were checked daily for a first open flower and the data is summarized in Table 3. In general, the TFL edited lines began flowering between 49 and 75 days after plugging (transfer from tissue culture to controlled environment). Control unedited events did not flower during the 230 day evaluation. In addition to showing the early flowering phenotype, the edited TFL lines also show continuous flowering. The TFL edited lines consistently developed new branches which ended in a terminal flower and further developed several flowers and fruit at subtending nodes. In the TFL lines, the flowering would then commence on another emerging branch.

TABLE 4
Early flowering phenotype in TFL edited blackberry
plantlets in the E0 generation.

| | Plugging | Days to | |
|---|---|---|---|
| CE ID | Date | Flowering | TFL Alleles |
| CE119211 | Jan. 26, 2022 | 49 | see Table 3 |
| CE120701 | Feb. 10, 2022 | 57 | see Table 3 |
| CE120713 | Feb. 10, 2022 | 79 | see Table 3 |
| CE125667 | Mar. 24, 2022 | 71 | see Table 3 |
| CE145602 | Jul. 11, 2022 | 54 | see Table 3 |
| CE150427 | Aug. 4, 2022 | 75 | Alternate alleles, all |
| | | | copies edited |
| CE149714 | Jul. 28, 2022 | Did not flower | No Editing/control line |
| CE149716 | Jul. 28, 2022 | Did not flower | No editing/control line |
| CE149718 | Jul. 28, 2022 | Did not flower | No editing/control line |

### Example 5. Inheritance of Edited Alleles CE119211

CE 119211 was self-pollinated and the E1 generation of seeds were collected from fruit. The E1 seeds were germinated to generate E1 plants. These E1 progeny were screened for edits, and each individual had a combination of the edits detected in the parent plant CE119211, indicating that the edited alleles were heritable. Time to first flowering was evaluated in the E1 population and the observations are summarized in Table 5. Twenty-four of the plants flowered during a 270-day evaluation period, and nine did not flower during this time period. The earliest observation of flowering occurred at 70 days after transfer to soil, and the latest instance of flowering in a plant where flowering was detected was at 163 days after transfer. The average time to flowering among flowering plants was 109 days.

The three individuals of CE150473, CE150482 and CE150489 were shown by sequencing to all have the same combination of edited alleles of TFL; however, these three lines also showed significant differences in time to flowering. CE150473, CE150482, flowered at 70 and 129 days, and CE150489 did not flower. Because the parent plant CE119211 is not inbred, background genetics vary among these progeny even though the edited alleles of TFL were the same. These results indicate that the phenotypic expression of the edits can be impacted by genetic variation at other loci and/or environmental factors.

TABLE 5
Early flowering phenotypes among progeny of a TFL edited parent.

| CE ID | Plugging Date | Days to Flowering |
|---|---|---|
| CE150469 | Jun. 21, 2022 | 101 |
| CE150470 | Jun. 21, 2022 | 163 |
| CE150471 | Jun. 21, 2022 | 108 |
| CE150472 | Jun. 21, 2022 | Did not flower |
| CE150473 | Jun. 21, 2022 | 70 |
| CE150474 | Jun. 21, 2022 | 75 |
| CE150475 | Jun. 21, 2022 | 75 |
| CE150476 | Jun. 21, 2022 | 78 |
| CE150477 | Jun. 21, 2022 | Did not flower |
| CE150478 | Jun. 21, 2022 | 161 |
| CE150479 | Jun. 21, 2022 | 131 |
| CE150480 | Jun. 21, 2022 | 71 |
| CE150481 | Jun. 21, 2022 | Did not flower |
| CE150482 | Jun. 21, 2022 | 129 |
| CE150483 | Jun. 21, 2022 | 104 |
| CE150484 | Jun. 21, 2022 | 105 |
| CE150485 | Jun. 21, 2022 | 116 |
| CE150486 | Jun. 21, 2022 | 86 |
| CE150487 | Jun. 21, 2022 | 90 |
| CE150488 | Jun. 21, 2022 | 87 |
| CE150489 | Jun. 21, 2022 | Did not flower |
| CE150490 | Jun. 21, 2022 | Did not flower |
| CE150491 | Jun. 21, 2022 | Did not flower |
| CE150492 | Jun. 21, 2022 | 85 |
| CE150493 | Jun. 21, 2022 | 172 |
| CE150494 | Jun. 21, 2022 | 70 |
| CE150495 | Jun. 21, 2022 | Did not flower |
| CE150496 | Jun. 21, 2022 | Did not flower |
| CE150497 | Jun. 21, 2022 | 90 |
| CE150498 | Jun. 21, 2022 | Did not flower |
| CE150499 | Jun. 21, 2022 | 84 |
| CE150500 | Jun. 21, 2022 | 94 |
| CE150502 | Jun. 21, 2022 | Did not flower |

### Example 6. Yield Evaluation

The edited blackberry line C plants CE119211, CE120701, CE120713, and unedited control plants were vegetatively propagated via rooted cuttings. After rooting and establishment, the resulting plug plants were organized into a 4-plant-plot design and grown in a temperature-controlled greenhouse which would provide inductive conditions for flowering. This greenhouse trial was planted Jun. 15, 2022, and it included three plots of CE119211, four plots of CE120701, four plots of CE120713, and four plots of unedited control. All plants were monitored from June 15^{th} through the end of the trial on February 20 the following year. In particular, plants were evaluated for fruit set and the harvest of fruit. Marketable yield was calculated by harvesting berries, collecting data on berry mass, and discarding malformed or poorly pollinated fruit and the data collected is summarized in Table 6. All edited lines showed an earlier first harvest date, ranging from 17 to 38 days earlier than the first harvest from the unedited control plot. One of the four unedited control plots did not produce fruit during the harvest period, and one only produced fruit on the final harvest day. Total marketable yield was calculated by dividing the total fruit weight by the number of plants that were marked as alive to generate the plot plant average. The plot plant averages were then averaged over all 4 plots. All edited lines set marketable fruit earlier than the control plants. These results indicate that the early flowering trait resulting from TFL editing results in early fruit set and higher yield early during early plant development, relative to the primocane flowering but non-TFL edited baseline germplasm.

TABLE 6
Marketable yield in average grams per plot per plant

| | 2022- | 2022- | 2022- | 2022- | 2023- | 2023- | 2023- | 2023- | 2023- | 2023- | 2023- | 2023- |
|---|---|---|---|---|---|---|---|---|---|---|---|---|
| | W49 | W50 | W51 | W52 | W1 | W2 | W3 | W4 | W5 | W6 | W7 | W8 |
| CE119211 | 3.1 | 0 | 12.7 | 18.2 | 11 | 26.7 | 39 | 71.4 | 115.6 | 92.3 | 79 | 85 |
| CE120701 | | | 3 | 6 | 15.3 | 14.1 | 25.8 | 77 | 89.4 | 69 | 59.1 | 77.8 |
| CE120713 | | | | 0.6 | 2 | 8.9 | 20.4 | 46.9 | 119.5 | 105.3 | 97.4 | 90.1 |
| control | | | | | | 9.8 | 16.4 | 11.1 | 52.7 | 84.6 | 95.5 | 100.5 |

### Example 7. Edited TFL in Floricane Blackberry Line B Variety

An edited plant from floricane-flowering genotype blackberry line B was recovered as described in Example 1. The blackberry line B edited line contained an out-of-frame deletion in Exon 3, creating an early stop codon and a truncated protein sequence for one of the four alleles of TFL. The edited plant was self-pollinated to generate E1 seed and the E1 plants were screened to identify E1 lines with two edited copies of TFL. The selected E1 plant was self-pollinated to generate an E2 population of plants that segregated for 0, 1, 2, 3, and 4 edited copies of TFL. E2 plants with 0, 1, 2, 3, and 4 edited alleles were grown in greenhouse conditions for about a five-month period, and none of the plants exhibited an early flowering phenotype during this time period. These results indicate that background genetics (i.e., floricane versus primocane background), number of edited copies, or position of the edits may all influence expression of an early flowering phenotype.

### Example 8. Indoor Blackberry Fruit Production

The growth habit of four copy TFL-edited blackberry line C plants as described in Table 3, were compared to control or wild type plants. Typical blackberry plants show strong apical dominance, where a single cane shows strong apical dominance and little branching. Over time in production settings, additional canes or branches may emerge from the base of the cane, and canes will be trellised and pruned to facilitate harvest or affect fruiting lateral development and timing. Blackberry plants with a primocane trait have a determinate growth habit in which the cane growth will terminate in an inflorescence, and subtending nodes on ˜50% of the cane will have bud break from axillary meristems, which then also develop inflorescences.

Four copy TFL edited plants have altered architecture when compared to control plants, where the four copy TFL edited plants have a primary cane that develops and terminates in a flower earlier in development and with less vegetative growth of the cane than the control/wild type plants. Additionally, the early transition to flowering observed in the four copy TFL edited plants, resulted in a shorter, early flowering cane. We also observed earlier lateral bud break from these edited plants, where axillary meristems began to grow new branches prior to the observation of the terminal bud forming. As flower development begins on the first cane, these newly developed branches also transition to flower development. Over time, new branches also develop from the base of the cane or crown, which are progressively longer than the initial canes, but also transition to flowering. The ultimate effect is a shorter plant with bushy growth habit, which has continuous flower development on newly developing branches. This growth habit has been observed in multiple plants evaluated over the course of a year in greenhouse conditions, indicating the TFL edits also reduce the requirement of environmental signals to permit flowering.

The resulting determinate, bushy habit of the TFL edited lines facilitates indoor fruit production using the TFL edited lines. The determinate habit allows for growth in a closed environment as the height of the canes is overall shorter. Additionally, the continuous production of new canes which flower produces a bushy habit that is also conducive to indoor growth as the plant remains compact. The indoor growth environment can provide control of environmental conditions such that the plants do not experience a freeze or other conditions which would cause them to cease to flower. The TFL edited lines appear to have an everbearing phenotype that enables the use of these lines in indoor fruit production.

The foregoing is illustrative of the present invention and is not to be construed as limiting thereof. The invention is defined by the following claims, with equivalents of the claims to be included therein.

1. A Rosaceae plant or part thereof comprising at least one (e.g., one or more) mutation in an endogenous TERMINAL FLOWER (TFL) gene (e.g., one or more TFL genes) encoding a TFL polypeptide, wherein the endogenous gene encoding a TFL polypeptide comprises a sequence having at least 80% (e.g., 80, 81, 82, 83, 84, 85, 86, 87, 88, 89, 90, 91, 92, 93, 94, 95, 96, 97, 98, 99, or 100%) sequence identity to any one of the nucleotide sequences of SEQ ID NOs:72, 73, 106, 107, 114, 115, 126, 127, 140, 141, 153, 162, 163, 172, 173, 184, 185, 214, 215, 234, 235, or 252, comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, encodes a TFL polypeptide having at least 80% sequence identity to any one of the amino acid sequences of SEQ ID NOs:74, 108, 116, 128, 142, 154, 164, 174, 186, 216, 236 or 253 and/or encodes a region having at least 80% sequence identity to any one of the amino acid sequences of SEQ ID NOs:193-197, 247, 248, or 271-276.

2. (canceled)

3. The Rosaceae plant or part thereof of claim 1, wherein the at least mutation is a base substitution, a base deletion and/or a base insertion.

4-5. (canceled)

6. The Rosaceae plant or part thereof of claim 1, wherein the at least one mutation is a base deletion of about 1 base pair to about 100 consecutive base pairs, about 3 base pairs to about 2600 base pairs, or about 3 base pairs to about 519 base pairs.

7. (canceled)

8. The Rosaceae plant or part thereof of claim 6, wherein the at least one mutation in an endogenous TFL gene results in a truncated TFL polypeptide and/or no detectable TFL polypeptide.

9-11. (canceled)

12. The Rosaceae plant or part thereof of claim 1, wherein the Rosaceae plant or part thereof comprises at least two endogenous TFL genes and at least one allele of the at least two endogenous TFL genes comprises the at least one (e.g., one or more) mutation.

13. (canceled)

14. The Rosaceae plant or part thereof of claim 1, wherein the Rosaceae plant comprising the at least one mutation exhibits a phenotype of reduced time to flowering, a longer duration of flowering, a more determinate plant growth pattern and/or one or more improved yield characteristics compared to a control Rosaceae plant.

15. The Rosaceae plant or part thereof of claim 1, wherein the Rosaceae plant is a caneberry or stone fruit, optionally wherein the Rosaceae plant is a blackberry, a black raspberry, a cherry, a plum or a peach.

16. (canceled)

17. The Rosaceae plant or part thereof of claim 1, wherein the at least one mutation results in a mutated TFL1 gene having at least 90% sequence identity to any one of the nucleotide sequences of SEQ ID NO:277-284, 295, 297, 298, 300, 302, 304, 306, 308, 309, 310, 311, or 313 and/or encoding a mutated TFL1 polypeptide having at least 90% sequence identity to any one of the amino acid sequences of SEQ ID NOs:285-291, 296, 299, 301, 303, 305, 307, or 312.

18-35. (canceled)

36. A Rosaceae plant regenerated from the Rosaceae plant part of claim 1.

37-63. (canceled)

64. A method for producing a Rosaceae plant or part thereof comprising at least one cell having an endogenous TFL gene with a mutation, the method comprising contacting a target site in an endogenous TFL gene in the Rosaceae plant or part with a nuclease comprising a cleavage domain and a DNA-binding domain, wherein the nucleic acid binding domain binds to a target site in the endogenous TFL gene, wherein the endogenous TFL gene
(a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252,
(b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270,
(c) encodes an amino acid sequence having at least 80% sequence identity to any one of SEQ ID NOs:74, 108, 116, 128, 142, 154, 164, 174, 186, 216, 236 or 253, and/or
(d) encodes a region having at least 80% sequence identity to any one of the amino acid sequences of SEQ ID NOs:193-197, 247, 248, or 271-276, thereby producing the Rosaceae plant or part thereof comprising at least one cell having an endogenous TFL gene with a mutation.

65-68. (canceled)

69. The method of claim 64, wherein the mutation is a deletion.

70. The method claim 69, wherein the deletion results in a truncated TFL polypeptide and/or no detectable TFL polypeptide.

71. (canceled)

72. The method of claim 70, wherein the truncated TFL polypeptide comprises a C-terminal truncation comprising a deletion of at least 1 amino acid residue.

73. The method of claim 64, wherein the mutation is a deletion of at least 3 base pairs (e.g., at least 3 consecutive base pairs to about 516 consecutive base pairs or at least 3 consecutive base pairs to about 2600 consecutive base pairs) from a sequence having at least 80% sequence identity to the nucleotide sequence of any one of SEQ ID NOs:72, 73, 106, 107, 114, 115, 126, 127, 140, 141, 153, 162, 163, 172, 173, 184, 185, 214, 215, 234, 235, or 252.

74-75. (canceled)

76. The method of claim 64, wherein the at least one mutation results in a mutated TFL gene having at least 90% sequence identity to any one of SEQ ID NO:277-284, 295, 297, 298, 300, 302, 304, 306, 308, 309, 310, 311, or 313 and/or encoding a mutated TFL1 polypeptide having at least 90% sequence identity to any one of SEQ ID NOs:285-291, 296, 299, 301, 303, 305, 307, or 312.

77. A plant produced by the method of claim 64.

78. A guide nucleic acid that binds to a target site in a TFL gene, wherein the target site is in a region of the TFL gene having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270 optionally wherein the guide nucleic acid comprises a spacer having the nucleotide sequence of any one of SEQ ID NOs:198-210, 211-213 or 249-251.

79-87. (canceled)

88. A mutated endogenous TFL gene, wherein mutated endogenous TFL gene comprises a nucleic acid sequence having at least 90% sequence identity to any one of SEQ ID NO:277-284, 295, 297, 298, 300, 302, 304, 306, 308, 309, 310, 311, or 313 and/or encodes a mutated TFL1 polypeptide having at least 90% sequence identity to any one of SEQ ID NOs:285-291, 296, 299, 301, 303, 305, 307, or 312, optionally wherein the mutated endogenous TFL gene comprises a non-natural mutation.

89. (canceled)

90. A Rosaceae plant or part thereof comprising the mutated endogenous TFL gene of claim 88.

91. The Rosaceae plant of claim 90, wherein the Rosaceae plant exhibits a phenotype of a reduced time to flowering, a longer duration of flowering, a more determinate plant growth pattern and/or one or more improved yield characteristics compared to a control Rosaceae plant.

92-96. (canceled)
